SEQUENCE LISTING (239 sequences)

SEQ ID NO: 1 - protein, 119 aa, Artificial Sequence
  Synthetic Construct
QVQLQQSGGGLVQPGGSLRLSCTASGFTFSSYAMSWVRQAPGKGLEWVSAISGSGGSTYYADSVKGRFTISRDNSKNTLYLQMNSLRVEDTAVYYCARYSIGRHTFDHWGQGTLVTVSA

SEQ ID NO: 2 - protein, 111 aa, Artificial Sequence
  Synthetic Construct
NFMLTQPHSVSESPGKTVTISCTRSSGGIASNFVQWYQQRPGSVPTTVIYRDNQRPSGVPDRFSGSVDSSSNSASLTISGLKTDDEADYYCQSYDDHNHWVFGGGTKLTVL

SEQ ID NO: 3 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 4 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLHAEDVAIYYCQQYYTPPLAFGGGTKLEIK

SEQ ID NO: 5 - protein, 118 aa, Artificial Sequence
  Synthetic Construct
QVQLQQSGGGLVQPGGSLRLSCAASGFTFSGYAMTWVRQAPGKGLEWVSAITSTGGRTYYADSVKGRFTTSRDNSRNTLYLQMNSLRAEDTAVYYCARESNFRAFDIWGQGTMVTVSA

SEQ ID NO: 6 - protein, 112 aa, Artificial Sequence
  Synthetic Construct
EIVLTQSPLSLPVTPGEPASISCRSSQSLLHSNGYNYLDWYLQKPGQSPQLLIYLNSNRASGVPDRFSGSGSGTDFTLQISRVEAEDVGVYYCMQALQIPPTFGGGTKVDIK

SEQ ID NO: 7 - protein, 118 aa, Artificial Sequence
  Synthetic Construct
EVQLVESGGGLVQPGGSLRLSCAASGFTFIDAWMTWVRQAPGKGLEWVSVIYSGGSTYYADSVKGRFTISRDNSKNTLYLQMNSLRAEDTAVYYCARGARGHPGQDYWGQGTLVTVSA

SEQ ID NO: 8 - protein, 112 aa, Artificial Sequence
  Synthetic Construct
NFMLTQPHSVSESPGKTVTISCTRSSGTIASNFVQWYQQRPGSSPTPVIFENDRRPSGVPDRFSGSIDSSSNSASLTISSLNTEDKADYYCQSYDSSTHGWVFGGGTQLTVL

SEQ ID NO: 9 - protein, 117 aa, Artificial Sequence
  Synthetic Construct
QVNLRESGGGLVQPGGSLRLSCAASGFTFSDYYMSWIRQAPGGGLEWVSYTSRFGSDTNYADSVKGRFTISRDNVQNSLYLQMNSLRAEDTAVYYCVRDVHNRDAYWGQGTLVTVSA

SEQ ID NO: 10 - protein, 110 aa, Artificial Sequence
  Synthetic Construct
SYVLTQPPSASGTPGQRVTISCSGSSSNIGGNSVSWYQQLPGTAPKLLIYRNHQRPSGVPDRFSGSKSGTSASLAISGLRSDDEADYYCATWDFSLSGFVFGTGTKVTVL

SEQ ID NO: 11 - protein, 108 aa, Artificial Sequence
  Synthetic Construct
QVQLVQSGGGLVQPGGSLRLSCAASGFTFSSYAMSWVRQAPGKGLEWVSAISGSGGSTYYADSVKGRFTISRDNSKNTYYCANTDYYDSSSHTPADYWGQGTLVTVSA

SEQ ID NO: 12 - protein, 108 aa, Artificial Sequence
  Synthetic Construct
ETTLTQSPSSLSASVGDRVTITCRASQDIRNDLDWFQQKPGEAPKRLISAASNLQSGVPSRFSGGGSGSEFTLTIHSLESEDFATYYCQQSYITPPWTFGQGTKLEIK

SEQ ID NO: 13 - protein, 122 aa, Artificial Sequence
  Synthetic Construct
QVQLQESGGGLVQPGGSLRLSCAASGFTFSSYGMSWVRQAPGKGLEWVSTISGSGSSTNYADSVKGRFTISRDNSKNTLFLQMNSLRAEDTAVYFCAKGRYYYDSLDAFDIWGQGTMVTVSA

SEQ ID NO: 14 - protein, 108 aa, Artificial Sequence
  Synthetic Construct
EIVLTQSPGTLSLSPGERATLSCRASQEIRTAYLAWYQQKPGQAPRLLIYYASSRATGIPDRFSGSRSGTDFTLTISRLEPEDFAVYSCQQYDTSPPTFGQGTRLEIK

SEQ ID NO: 15 - protein, 122 aa, Artificial Sequence
  Synthetic Construct
QVQLVESGGGLVQPGGSLRLSCAASGFTFSSYAMSWVRQAPGKGLEWVSAISGTGGSTYYADSVKGRFTISRDNSKNTLYLQMNSLRAEDTAAYYCAKDKWSSWPTYYFDYWGQGTLVTVSA

SEQ ID NO: 16 - protein, 110 aa, Artificial Sequence
  Synthetic Construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVIFGGGTKVTVL

SEQ ID NO: 17 - protein, 122 aa, Artificial Sequence
  Synthetic Construct
QVQLQVSGGGLVQPGGSLRLSCAASGFTFSSYSMAWVRQAPGKGLEWVAAVSNSGVETYYADSVKGRFTISRDNSKNTLYLQMNSLRAEDTAVYYCAKRTRQLLTPREFDYWGQGTLVTVLA

SEQ ID NO: 18 - protein, 107 aa, Artificial Sequence
  Synthetic Construct
ETTLTQSPSSVSASVGDRVTLTCRASQDITRWLAWYQQKPGKAPKLLIYDASSLQSGVPSRFSGSGSGTDFTLTISSLQPEDFATYYCQQGSSVPFTFGGGTKVEIK

SEQ ID NO: 19 - protein, 117 aa, Artificial Sequence
  Synthetic Construct
QVNLRESGGGLIQPGGSLRLSCAASGFTFTNYAMSWVRQAPGKGLEWVSSVSSAGGSTYYADSVKGRFTISRDNAKNSLYLQMNSLRAEDTAVYYCARRVNRAFDLWGRGTLVTVSA

SEQ ID NO: 20 - protein, 109 aa, Artificial Sequence
  Synthetic Construct
VVMTQSPGTLSLSPGERATLSCRASQSVSSSYLAWYQQKPGQAPRLLIYGASSRATGIPDRFSGSGSGTDFTLTISRLEPEDFAVYYCQQYGSSPPMYTFGQGTKLEIK

SEQ ID NO: 21 - protein, 121 aa, Artificial Sequence
  Synthetic Construct
QVQLQESGGGLVQPGGSLRLSCAASGFTFSNAWMSWVRQAPGKGLVWVGRIKSKTDGGTTDYAAPVKGRFTISRDDSKNTLYLQMNSLKTEDTAVYYCTTDKSYGYTFDYWGQGTLVTVSA

SEQ ID NO: 22 - protein, 110 aa, Artificial Sequence
  Synthetic Construct
SYVLTQPPSASGTPGQRVTISCSGSGSNIGSNSVHWYQQLPGTAPKLLIYTNNQRPSGVPDRFSGSKSGISASLAISGLQSEDEAVYYCATWDDRLSGPVFAAGTKLTVL

SEQ ID NO: 23 - protein, 119 aa, Artificial Sequence
  Synthetic Construct
QVNLRESGGGLVKPGGSLRLSCAASGFTFSSYWMHWVRQAPGKGLEWVSAISGSGAGTYYPDSVKGRFTISRDNSKNTLYLQMNSLRAEDTAVYYCARDRSLSFGFDIWGQGTLVSVSG

SEQ ID NO: 24 - protein, 110 aa, Artificial Sequence
  Synthetic Construct
NFMLTQPHSVSGSPGKTVTISCTRSSGSIGSTYVQWYQQRPGSPPTTVIYKDDQRPSGVPDRFSGSIDGSSNSASLTISGLETEDEADYYCQSSDTSNLVFGGGTKVTVL

SEQ ID NO: 25 - protein, 121 aa, Artificial Sequence
  Synthetic Construct
QVQLQQSGGGLVQPGGSLRLSCAAPGFTFSRYWMSWVRQAPGKGLEWVANIKGDGSQTYYADSVKGRFTISRDNAMKTVYLQMNSLRAEDTAIYYCAKGAAYHINSWLDPWGQGTLVTVSA

SEQ ID NO: 26 - protein, 108 aa, Artificial Sequence
  Synthetic Construct
ETTLTQSPGTLSVSPGERVTLSCRASQSISGNYLAWYQQRPGQAPRLLIYGAFRRATGIPDRFSGSGSGTDFTLTITRLEPEDFATYYCQHYNNFPHTFGAGTKVDIK

SEQ ID NO: 27 - protein, 118 aa, Artificial Sequence
  Synthetic Construct
KVQLVESGGGLVKPGGSLRLSCAASGITFKHAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 28 - protein, 113 aa, Artificial Sequence
  Synthetic Construct
DIVMTQSPDSLAVSLGERATINCKSSQSVLYQVNNRNYLAWYQQKPGQPPKLLINQASTRASGVPDRFSGSGSGTEFTLIISSLQAEDVAIYYCQQYYTPPLAFGGGTKLEIK

SEQ ID NO: 29 - protein, 118 aa, Artificial Sequence
  Synthetic Construct
KVQLVESGGGLVKPGGSLRLSCAASGFTFNNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 30 - protein, 113 aa, Artificial Sequence
  Synthetic Construct
DIVMTQSPDSLAVSLGERATINCKSSQTVLYPLNNRNYLAWYQQKPGQPPKLLINQASTRASGVPDRFSGSGSGTEFTLIISSLQAEDVAIYYCQQYYTPPLAFGGGTKLEIK

SEQ ID NO: 31 - protein, 118 aa, Artificial Sequence
  Synthetic Construct
KVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 32 - protein, 113 aa, Artificial Sequence
  Synthetic Construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 33 - protein, 118 aa, Artificial Sequence
  Synthetic Construct
KVQLVESGGGLVKPGGSLRLSCAASGFTFPNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 34 - protein, 113 aa, Artificial Sequence
  Synthetic Construct
DIVMTQSPDSLAVSLGERATINCKSSQSVLYPGNNRNYLAWYQQKPGQPPKLLINQASTRASGVPDRFSGSGSGTEFTLIISSLQAEDVAIYYCQQYYTPPLAFGGGTKLEIK

SEQ ID NO: 35 - protein, 118 aa, Artificial Sequence
  Synthetic Construct
KVQLVESGGGLVKPGGSLRLSCAASGLTFGNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 36 - protein, 113 aa, Artificial Sequence; identical to SEQ ID NO: 34
  Synthetic Construct

SEQ ID NO: 37 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGNHSSDIWGQGTMVTVSA

SEQ ID NO: 38 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYYTPPLAFGGGTKLEIK

SEQ ID NO: 39 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGAHSSDIWGQGTMVTVSA

SEQ ID NO: 40 - protein, 113 aa, Artificial Sequence; identical to SEQ ID NO: 38
  Synthetic Construct

SEQ ID NO: 41 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGQHSSDIWGQGTMVTVSA

SEQ ID NO: 42 - protein, 113 aa, Artificial Sequence; identical to SEQ ID NO: 38
  Synthetic Construct

SEQ ID NO: 43 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDAWGQGTMVTVSA

SEQ ID NO: 44 - protein, 113 aa, Artificial Sequence; identical to SEQ ID NO: 38
  Synthetic Construct

SEQ ID NO: 45 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDSWGQGTMVTVSA

SEQ ID NO: 46 - protein, 113 aa, Artificial Sequence; identical to SEQ ID NO: 38
  Synthetic Construct

SEQ ID NO: 47 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSDRASDKWGQGTMVTVSA

SEQ ID NO: 48 - protein, 113 aa, Artificial Sequence; identical to SEQ ID NO: 38
  Synthetic Construct

SEQ ID NO: 49 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDTWGQGTMVTVSA

SEQ ID NO: 50 - protein, 113 aa, Artificial Sequence; identical to SEQ ID NO: 38
  Synthetic Construct

SEQ ID NO: 51 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGNHSQDIWGQGTMVTVSA

SEQ ID NO: 52 - protein, 113 aa, Artificial Sequence; identical to SEQ ID NO: 38
  Synthetic Construct

SEQ ID NO: 53 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGQHSQDIWGQGTMVTVSA

SEQ ID NO: 54 - protein, 113 aa, Artificial Sequence; identical to SEQ ID NO: 38
  Synthetic Construct

SEQ ID NO: 55 - protein, 118 aa, Artificial Sequence
  Synthetic Construct
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGAHSQDIWGQGTMVTVSA

SEQ ID NO: 56 - protein, 113 aa, Artificial Sequence; identical to SEQ ID NO: 38
  Synthetic Construct

SEQ ID NO: 57 - protein, 118 aa, Artificial Sequence; identical to SEQ ID NO: 3
  Synthetic Construct

SEQ ID NO: 58 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLTPPLAFGGGTKLEIK

SEQ ID NO: 59 - protein, 118 aa, Artificial Sequence; identical to SEQ ID NO: 3
  Synthetic Construct

SEQ ID NO: 60 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLRPPLNFGGGTKLEIK

SEQ ID NO: 61 - protein, 118 aa, Artificial Sequence; identical to SEQ ID NO: 3
  Synthetic Construct

SEQ ID NO: 62 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLTPPLNFGGGTKLEIK

SEQ ID NO: 63 - protein, 118 aa, Artificial Sequence; identical to SEQ ID NO: 3
  Synthetic Construct

SEQ ID NO: 64 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQNYLTPPLSFGGGTKLEIK

SEQ ID NO: 65 - protein, 118 aa, Artificial Sequence; identical to SEQ ID NO: 3
  Synthetic Construct

SEQ ID NO: 66 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLKAPLAFGGGTKLEIK

SEQ ID NO: 67 - protein, 118 aa, Artificial Sequence; identical to SEQ ID NO: 3
  Synthetic Construct

SEQ ID NO: 68 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLNAPLHFGGGTKLEIK

SEQ ID NO: 69 - protein, 118 aa, Artificial Sequence; identical to SEQ ID NO: 3
  Synthetic Construct

SEQ ID NO: 70 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLEAPLVFGGGTKLEIK

SEQ ID NO: 71 - protein, 118 aa, Artificial Sequence; identical to SEQ ID NO: 3
  Synthetic Construct

SEQ ID NO: 72 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLKAPLHFGGGTKLEIK

SEQ ID NO: 73 - protein, 118 aa, Artificial Sequence; identical to SEQ ID NO: 3
  Synthetic Construct

SEQ ID NO: 74 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQRLIAPPFTFGGGTKLEIK

SEQ ID NO: 75 - protein, 118 aa, Artificial Sequence; identical to SEQ ID NO: 3
  Synthetic Construct

SEQ ID NO: 76 - protein, 113 aa, Artificial Sequence
  Synthetic Construct
DIVMTQSPDSLAVSLGERATINCKSSQSVLYSSNNRNYLAWYQQKPGQPPKLLINQASTRASGVPDRFSGSGSGTEFTLIISSLQAEDVAIYYCQNYLTPPLAFGGGTKLEIK

SEQ ID NO: 77 - protein, 120 aa, Artificial Sequence
  Synthetic Construct
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSS

SEQ ID NO: 78 - protein, 107 aa, Artificial Sequence
  Synthetic Construct
DIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIK

SEQ ID NO: 79 - protein, 118 aa, Artificial Sequence
  Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 80 - protein, 113 aa, Artificial Sequence; identical to SEQ ID NO: 32
  Synthetic Construct

SEQ ID NO: 81 - protein, 5 aa, Artificial Sequence
  Synthetic Construct
RAWMN

SEQ ID NO: 82 - protein, 19 aa, Artificial Sequence
  Synthetic Construct
RIKRKTDGETTDYAAPVKG

SEQ ID NO: 83 - protein, 7 aa, Artificial Sequence
  Synthetic Construct
SNRAFDI

SEQ ID NO: 84 - protein, 17 aa, Artificial Sequence
  Synthetic Construct
KSSQSVLYAGNNRNYLA

SEQ ID NO: 85 - protein, 7 aa, Artificial Sequence
  Synthetic Construct
QASTRAS

SEQ ID NO: 86 - protein, 9 aa, Artificial Sequence
  Synthetic Construct
QQYYTPPLA

SEQ ID NO: 87 - protein, 111 aa, Artificial Sequence
  Synthetic Construct
DIVLTQSPASLAVSLGQRATISCKASQSVDYDGDSYMDWYQQKPGQPPKLLIYAASNLESGIPARFSGSGSGTDFTLNIHPVEEEDAATYYCQQTHEDPWTFGGGTKLEIK

SEQ ID NO: 88 - protein, 111 aa, Artificial Sequence
  Synthetic Construct
DIVLTQSPASLAVSLGQRATISYRASKSVSTSGYSYMHWNQQKPGQPPRLLIYLVSNLESGVPARFSGSGSGTDFTLNIHPVEEEDAATYYCQQTHEDPWTFGGGTKLEIK

SEQ ID NO: 89 - protein, 111 aa, Artificial Sequence
  Synthetic Construct
DIVLTQSPASLAVSLGQRATISYRASKSVSTSGYSYMHWNQQKPGQPPKLLIYAASNLESGIPARFSGSGSGTDFTLNIHPVEEEDAATYYCQQTHEDPWTFGGGTKLEIK

SEQ ID NO: 90 - protein, 111 aa, Artificial Sequence
  Synthetic Construct
DIVLTQSPASLAVSLGQRATISYRASKSVSTSGYSYMHWNQQKPGQPPRLLIYLVSNLESGIPARFSGSGSGTDFTLNIHPVEEEDAATYYCQQTHEDPWTFGGGTKLEIK

SEQ ID NO: 91 - protein, 111 aa, Artificial Sequence
  Synthetic Construct
DIVLTQSPASLAVSLGQRATISCKASQSVDYDGDSYMDWYQQKPGQPPRLLIYLVSNLESGIPARFSGSGSGTDFTLNIHPVEEEDAATYYCQQTHEDPWTFGGGTKLEIK

SEQ ID NO: 92 - protein, 111 aa, Artificial Sequence; identical to SEQ ID NO: 88
  Synthetic Construct

SEQ ID NO: 93 - protein, 118 aa, Artificial Sequence
  Synthetic Construct
EVQLQQSGPELEKPGASVKISCKASGYSFSDYNMNWVKQGNGESLEWIGYVDPYYGDTRYNQNFKGKATLTVDKSSSTAYMQLKSLTSEDSAVYYCALSETPRAMDYWGQGTSVTVSP

SEQ ID NO: 94 - protein, 107 aa, Artificial Sequence
  Synthetic Construct
DIVMTQSPATLSVTPGDRVSLSCRASQSISDYLHWYQQKSNESPRLLIKYASQSISGIPSRFSGSGSGSDFTLNINSVEPEDVGVYYCQNGHSLPLTFGAGTKLELK

SEQ ID NO: 95 - protein, 121 aa, Artificial Sequence
  Synthetic Construct
QIQLVQSGPELKKPGETVKISCKASGFTFTNYGMNWVKQAPGKGLKWMGWINTFTGEPTLADDFMGRFALSLETSASTAYLQINYLKHEDTATYFCARGGMGVRLRYFDVWGAGTTVTVSS

SEQ ID NO: 96 - protein, 323 aa, Artificial Sequence
  Synthetic Construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 97 - protein, 17 aa, Artificial Sequence
  Synthetic Construct
KSSQTVLYPLNNRNYLA

SEQ ID NO: 98 - protein, 7 aa, Artificial Sequence
  Synthetic Construct
SDRASDK

SEQ ID NO: 99 - protein, 9 aa, Artificial Sequence
  Synthetic Construct
QQYLTPPLN

SEQ ID NO: 100 - protein, 5 aa, Artificial Sequence
  Synthetic Construct
DYNMN

SEQ ID NO: 101 - protein, 5 aa, Artificial Sequence
  Synthetic Construct
NYGMN

SEQ ID NO: 102 - protein, 17 aa, Artificial Sequence
  Synthetic Construct
WINTYTGQPTHADDFKG

SEQ ID NO: 103 - protein, 12 aa, Artificial Sequence
  Synthetic Construct
GGMGVRLRYFDV

SEQ ID NO: 104 - protein, 15 aa, Artificial Sequence
  Synthetic Construct
KASQSVDYDGDSYMD

SEQ ID NO: 105 - protein, 7 aa, Artificial Sequence
  Synthetic Construct
AASNLES

SEQ ID NO: 106 - protein, 9 aa, Artificial Sequence
  Synthetic Construct
HQTNEDPWT

SEQ ID NO: 107 - protein, 15 aa, Artificial Sequence
  Synthetic Construct
RASKSVSTSGYSYMH

SEQ ID NO: 108 - protein, 7 aa, Artificial Sequence
  Synthetic Construct
LVSNLES

SEQ ID NO: 109 - protein, 9 aa, Artificial Sequence
  Synthetic Construct
HQTNEDPWT

SEQ ID NO: 110 - protein, 121 aa, Artificial Sequence
  Synthetic Construct
QIQLVQSGPELKKPGETVKISCKASGFTFTNYGMNWVRQAPGKGLKWMGWINTYTGQPTHADDFKGRFAFSLETSARTAFLEINSLQNEDTATYFCARGGMGVRLRYFDVWGAGTTVTVSS

SEQ ID NO: 111 - protein, 111 aa, Artificial Sequence
  Synthetic Construct
DIVLTQSPASLAVSLGQRATISCKASQSVDYDGDSYMDWYQQKPGQPPKLLIYAASNLESGIPARFSGSGSGTDFTLNIHPVEEEDAATYYCHQTNEDPWTFGGGTKLEIK

SEQ ID NO: 112 - protein, 111 aa, Artificial Sequence
  Synthetic Construct
DIVLTQSPASLAVSLGQRATISYRASKSVSTSGYSYMHWNQQKPGQPPRLLIYLVSNLESGVPARFSGSGSGTDFTLNIHPVEEEDAATYYCHQTNEDPWTFGGGTKLEIK

SEQ ID NO: 113 - protein, 111 aa, Artificial Sequence; identical to SEQ ID NO: 111
  Synthetic Construct

SEQ ID NO: 114 - protein, 111 aa, Artificial Sequence
  Synthetic Construct
DIVLTQSPASLAVSLGQRATISYRASKSVSTSGYSYMHWNQQKPGQPPRLLIYLVSNLESGIPARFSGSGSGTDFTLNIHPVEEEDAATYYCHQTNEDPWTFGGGTKLEIK

SEQ ID NO: 115 - protein, 5 aa, Artificial Sequence
  Synthetic Construct
SYAMS

SEQ ID NO: 116 - protein, 17 aa, Artificial Sequence
  Synthetic Construct
AISGSGGSTYYADSVKG

SEQ ID NO: 117 - protein, 10 aa, Artificial Sequence
  Synthetic Construct
YSIGRHTFDH

SEQ ID NO: 118 - protein, 13 aa, Artificial Sequence
  Synthetic Construct
TRSSGGIASNFVQ

SEQ ID NO: 119 - protein, 7 aa, Artificial Sequence
  Synthetic Construct
RDNQRPS

SEQ ID NO: 120 - protein, 10 aa, Artificial Sequence
  Synthetic Construct
QSYDDHNHWV

SEQ ID NO: 121 - protein, 5 aa, Artificial Sequence
  Synthetic Construct
NAWMN

SEQ ID NO: 122 - protein, 7 aa, Artificial Sequence
  Synthetic Construct
SNRAFDI

SEQ ID NO: 123 - protein, 17 aa, Artificial Sequence
  Synthetic Construct
KSSQSVLYSSNNRNYLA

SEQ ID NO: 124 - protein, 5 aa, Artificial Sequence
  Synthetic Construct
GYAMT

SEQ ID NO: 125 - protein, 17 aa, Artificial Sequence
  Synthetic Construct
AITSTGGRTYYADSVKG

SEQ ID NO: 126 - protein, 9 aa, Artificial Sequence
  Synthetic Construct
ESNFRAFDI

SEQ ID NO: 127 - protein, 16 aa, Artificial Sequence
  Synthetic Construct
RSSQSLLHSNGYNYLD

SEQ ID NO: 128 - protein, 7 aa, Artificial Sequence
  Synthetic Construct
LNSNRAS

SEQ ID NO: 129 - protein, 9 aa, Artificial Sequence
  Synthetic Construct
MQALQIPPT

SEQ ID NO: 130 - protein, 5 aa, Artificial Sequence
  Synthetic Construct
DAWMT

SEQ ID NO: 131 - protein, 16 aa, Artificial Sequence
  Synthetic Construct
VIYSGGSTYYADSVKG

SEQ ID NO: 132 - protein, 10 aa, Artificial Sequence
  Synthetic Construct
GARGHPGQDY

SEQ ID NO: 133 - protein, 13 aa, Artificial Sequence
  Synthetic Construct
TRSSGTIASNFVQ

SEQ ID NO: 134 - protein, 7 aa, Artificial Sequence
  Synthetic Construct
ENDRRPS

SEQ ID NO: 135 - protein, 11 aa, Artificial Sequence
  Synthetic Construct
QSYDSSTHGWV

SEQ ID NO: 136 - protein, 5 aa, Artificial Sequence
  Synthetic Construct
DYYMS

SEQ ID NO: 137 - protein, 17 aa, Artificial Sequence
  Synthetic Construct
YTSRFGSDTNYADSVKG

SEQ ID NO: 138 - protein, 8 aa, Artificial Sequence
  Synthetic Construct
DVHNRDAY

SEQ ID NO: 139 - protein, 13 aa, Artificial Sequence
  Synthetic Construct
SGSSSNIGGNSVS

SEQ ID NO: 140 - protein, 7 aa, Artificial Sequence
  Synthetic Construct
RNHQRPS

SEQ ID NO: 141 - protein, 11 aa, Artificial Sequence
  Synthetic Construct
ATWDFSLSGFV

SEQ ID NO: 142 - protein, 5 aa, Artificial Sequence
  Synthetic Construct
SYAMS

SEQ ID NO: 143 - protein, 17 aa, Artificial Sequence
  Synthetic Construct
AISGSGGSTYYADSVKG

SEQ ID NO: 144 - protein, 3 aa, Artificial Sequence
  Synthetic Construct
ADY

SEQ ID NO: 145 - protein, 11 aa, Artificial Sequence
  Synthetic Construct
RASQDIRNDLD

SEQ ID NO: 146 - protein, 7 aa, Artificial Sequence
  Synthetic Construct
AASNLQS

SEQ ID NO: 147 - protein, 10 aa, Artificial Sequence
  Synthetic Construct
QQSYITPPWT

SEQ ID NO: 148 - protein, 5 aa, Artificial Sequence
  Synthetic Construct
SYGMS

SEQ ID NO: 149 - protein, 17 aa, Artificial Sequence
  Synthetic Construct
TISGSGSSTNYADSVKG

SEQ ID NO: 150 - protein, 13 aa, Artificial Sequence
  Synthetic Construct
GRYYYDSLDAFDI

SEQ ID NO: 151 - protein, 12 aa, Artificial Sequence
  Synthetic Construct
RASQEIRTAYLA

SEQ ID NO: 152 - protein, 7 aa, Artificial Sequence
  Synthetic Construct
YASSRAT

SEQ ID NO: 153 - protein, 9 aa, Artificial Sequence
  Synthetic Construct
QQYDTSPPT

SEQ ID NO: 154 - protein, 17 aa, Artificial Sequence
  Synthetic Construct
AISGTGGSTYYADSVKG

SEQ ID NO: 155 - protein, 13 aa, Artificial Sequence
  Synthetic Construct
DKWSSWPTYYFDY

SEQ ID NO: 156 - protein, 13 aa, Artificial Sequence
  Synthetic Construct
TRSSGSIASNYVQ

SEQ ID NO: 157 - protein, 7 aa, Artificial Sequence
  Synthetic Construct
EDNQRPS

SEQ ID NO: 158 - protein, 9 aa, Artificial Sequence
  Synthetic Construct
QSYDSSNVI

SEQ ID NO: 159 - protein, 5 aa, Artificial Sequence
  Synthetic Construct
SYSMA

SEQ ID NO: 160 - protein, 17 aa, Artificial Sequence
  Synthetic Construct
AVSNSGVETYYADSVKG

SEQ ID NO: 161 - protein, 13 aa, Artificial Sequence
  Synthetic Construct
RTRQLLTPREFDY

SEQ ID NO: 162 - protein, 11 aa, Artificial Sequence
  Synthetic Construct
RASQDITRWLA

SEQ ID NO: 163 - protein, 7 aa, Artificial Sequence
  Synthetic Construct
DASSLQS

SEQ ID NO: 164 - protein, 9 aa, Artificial Sequence
  Synthetic Construct
QQGSSVPFT

SEQ ID NO: 165 - protein, 5 aa, Artificial Sequence
  Synthetic Construct
NYAMS

SEQ ID NO: 166 - protein, 17 aa, Artificial Sequence
  Synthetic Construct
SVSSAGGSTYYADSVKG

SEQ ID NO: 167 - protein, 8 aa, Artificial Sequence
  Synthetic Construct
RVNRAFDL

SEQ ID NO: 168 - protein, 12 aa, Artificial Sequence
  Synthetic Construct
RASQSVSSSYLA

SEQ ID NO: 169 - protein, 7 aa, Artificial Sequence
  Synthetic Construct
GASSRAT

SEQ ID NO: 170 - protein, 11 aa, Artificial Sequence
  Synthetic Construct
QQYGSSPPMYT

SEQ ID NO: 171 - protein, 5 aa, Artificial Sequence
  Synthetic Construct
NAWMS

SEQ ID NO: 172 - protein, 19 aa, Artificial Sequence
  Synthetic Construct
RIKSKTDGGTTDYAAPVKG

SEQ ID NO: 173 - protein, 10 aa, Artificial Sequence
  Synthetic Construct
DKSYGYTFDY

SEQ ID NO: 174 - protein, 13 aa, Artificial Sequence
  Synthetic Construct
SGSGSNIGSNSVH

SEQ ID NO: 175 - protein, 7 aa, Artificial Sequence
  Synthetic Construct
TNNQRPS

SEQ ID NO: 176 - protein, 11 aa, Artificial Sequence
  Synthetic Construct
ATWDDRLSGPV

SEQ ID NO: 177 - protein, 5 aa, Artificial Sequence
  Synthetic Construct
SYWMH

SEQ ID NO: 178 - protein, 17 aa, Artificial Sequence
  Synthetic Construct
AISGSGAGTYYPDSVKG

SEQ ID NO: 179 - protein, 10 aa, Artificial Sequence
  Synthetic Construct
DRSLSFGFDI

SEQ ID NO: 180 - protein, 13 aa, Artificial Sequence
  Synthetic Construct
TRSSGSIGSTYVQ

SEQ ID NO: 181 - protein, 7 aa, Artificial Sequence
  Synthetic Construct
KDDQRPS

SEQ ID NO: 182 - protein, 9 aa, Artificial Sequence
  Synthetic Construct
QSSDTSNLV

SEQ ID NO: 183 - protein, 5 aa, Artificial Sequence
  Synthetic Construct
RYWMS

SEQ ID NO: 184 - protein, 17 aa, Artificial Sequence
  Synthetic Construct
NIKGDGSQTYYADSVKG

SEQ ID NO: 185 - protein, 12 aa, Artificial Sequence
  Synthetic Construct
GAAYHINSWLDP

SEQ ID NO: 186 - protein, 12 aa, Artificial Sequence
  Synthetic Construct
RASQSISGNYLA

SEQ ID NO: 187 - protein, 7 aa, Artificial Sequence
  Synthetic Construct
GAFRRAT

SEQ ID NO: 188 - protein, 9 aa, Artificial Sequence
  Synthetic Construct
QHYNNFPHT

SEQ ID NO: 189 - protein, 5 aa, Artificial Sequence
  Synthetic Construct
HAWMN

SEQ ID NO: 190 - protein, 17 aa, Artificial Sequence
  Synthetic Construct
KSSQSVLYQVNNRNYLA

SEQ ID NO: 191 - protein, 17 aa, Artificial Sequence
  Synthetic Construct
KSSQSVLYPGNNRNYLA

SEQ ID NO: 192 - protein, 7 aa, Artificial Sequence
  Synthetic Construct
GNHSSDI

SEQ ID NO: 193 - protein, 7 aa, Artificial Sequence
  Synthetic Construct
GAHSSDI

SEQ ID NO: 194 - protein, 7 aa, Artificial Sequence
  Synthetic Construct
GQHSSDI

SEQ ID NO: 195 - protein, 7 aa, Artificial Sequence
  Synthetic Construct
SAYAFDA

SEQ ID NO: 196 - protein, 7 aa, Artificial Sequence
  Synthetic Construct
SAYAFDS

SEQ ID NO: 197 - protein, 7 aa, Artificial Sequence
  Synthetic Construct
SAYAFDT

SEQ ID NO: 198 - protein, 7 aa, Artificial Sequence
  Synthetic Construct
GNHSQDI

SEQ ID NO: 199 - protein, 7 aa, Artificial Sequence
  Synthetic Construct
GQHSQDI

SEQ ID NO: 200 - protein, 7 aa, Artificial Sequence
  Synthetic Construct
GAHSQDI

SEQ ID NO: 201 - protein, 7 aa, Artificial Sequence
  Synthetic Construct
SNRAFDI

SEQ ID NO: 202 - protein, 9 aa, Artificial Sequence
  Synthetic Construct
QQYLRPPLN

SEQ ID NO: 203 - protein, 9 aa, Artificial Sequence
  Synthetic Construct
QNYLTPPLS

SEQ ID NO: 204 - protein, 9 aa, Artificial Sequence
  Synthetic Construct
QQYLKAPLA

SEQ ID NO: 205 - protein, 9 aa, Artificial Sequence
  Synthetic Construct
QQYLNAPLH

SEQ ID NO: 206 - protein, 9 aa, Artificial Sequence
  Synthetic Construct
QQYLEAPLV

SEQ ID NO: 207 - protein, 9 aa, Artificial Sequence
  Synthetic Construct
QQYLKAPLH

SEQ ID NO: 208 - protein, 9 aa, Artificial Sequence
  Synthetic Construct
QRLIAPPFT

SEQ ID NO: 209 - protein, 9 aa, Artificial Sequence
  Synthetic Construct
QNYLTPPLA

SEQ ID NO: 210 - protein, 5 aa, Artificial Sequence
  Synthetic Construct
SYYMH

SEQ ID NO: 211 - protein, 17 aa, Artificial Sequence
  Synthetic Construct
EINPNNARINFNEKFKT

SEQ ID NO: 212 - protein, 11 aa, Artificial Sequence
  Synthetic Construct
GYYRYGAWFGY

SEQ ID NO: 213 - protein, 11 aa, Artificial Sequence
  Synthetic Construct
RASQDISDYLN

SEQ ID NO: 214 - protein, 7 aa, Artificial Sequence
  Synthetic Construct
YISRLHS

SEQ ID NO: 215 - protein, 9 aa, Artificial Sequence
  Synthetic Construct
QQGHTLPWT

SEQ ID NO: 216 - protein, 445 aa, Artificial Sequence
  Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 217 - protein, 444 aa, Artificial Sequence
  Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 218 - protein, 220 aa, Artificial Sequence
  Synthetic Construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 17 aa, Artificial Sequence
  Synthetic Construct
WINTFTGEPTLADDFMG

SEQ ID NO: 220 - protein, 9 aa, Artificial Sequence
  Synthetic Construct
QQTHEDPWT

SEQ ID NO: 221 - DNA, 363 bp, Artificial Sequence
  Synthetic Construct
cagatccagttggtgcagtctggacctgagctgaagaagcctggagagacagtcaagatctcctgcaaggcttctgggtttaccttcacaaactatggaatgaactgggtgaggcaggctccaggaaagggtttaaagtggatgggctggataaacacctacactggacaaccaacacatgctgatgacttcaagggacggtttgccttctctttggagacctctgccagaactgcctttttggagatcaacagcctccaaaatgaggacacggctacatatttctgtgcaagaggagggatgggggtacgactccggtacttcgatgtctggggcgcagggaccacggtcaccgtctcctca

SEQ ID NO: 222 - DNA, 333 bp, Artificial Sequence
  Synthetic Construct
gacattgtgctgacacagtctcctgcttccttagctgtgtctctagggcagagggccaccatctcctgcaaggccagccaaagtgttgattatgatggtgatagttatatggactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcatcaaactaatgaggatccgtggacgttcggtggaggcaccaagctggaaatcaaa

SEQ ID NO: 223 - DNA, 333 bp, Artificial Sequence
  Synthetic Construct
gacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcatcaaactaatgaggatccgtggacgttcggtggaggcaccaagctggaaatcaaa

SEQ ID NO: 224 - DNA, 333 bp, Artificial Sequence
  Synthetic Construct
gacattgtgctgacccaatctccagcttctttggctgtgtctctagggcagagggccaccatctcctgcaaggccagccaaagtgttgattatgatggtgatagttatatggactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcatcaaactaatgaggatccgtggacgttcggtggaggcaccaagctggaaatcaaa

SEQ ID NO: 225 - DNA, 333 bp, Artificial Sequence
  Synthetic Construct
gacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctgggatcccagccaggtttagtggcagtgggtctgggacagacttcaccctcaacatccatcctgtggaggaggaggatgctgcaacctattactgtcatcaaactaatgaggatccgtggacgttcggtggaggcaccaagctggaaatcaaa

SEQ ID NO: 226 - DNA, 333 bp, Artificial Sequence
  Synthetic Construct
gacattgtgctgacccaatctccagcttctttggctgtgtctctagggcagagggccaccatctcctgcaaggccagccaaagtgttgattatgatggtgatagttatatggattggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacttattactgtcagcaaactcatgaggatccgtggacgttcggtggaggcaccaagctggaaatcaaa

SEQ ID NO: 227 - DNA, 333 bp, Artificial Sequence
  Synthetic Construct
gacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacttattactgtcagcaaactcatgaggatccgtggacgttcggtggaggcaccaagctggaaatcaaa

SEQ ID NO: 228 - DNA, 333 bp, Artificial Sequence
  Synthetic Construct
gacattgtgctgacacagtctcctgcttccttagctgtatctctggggcagagggccaccatctcatacagggccagcaaaagtgtcagtacatctggctatagttatatgcactggaaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacttattactgtcagcaaactcatgaggatccgtggacgttcggtggaggcaccaagctggaaatcaaa

SEQ ID NO: 229 - DNA, 333 bp, Artificial Sequence
  Synthetic Construct
gacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctgggatcccagccaggtttagtggcagtgggtctgggacagacttcaccctcaacatccatcctgtggaggaggaggatgctgcaacttattactgtcagcaaactcatgaggatccgtggacgttcggtggaggcaccaagctggaaatcaaa

SEQ ID NO: 230 - DNA, 333 bp, Artificial Sequence
  Synthetic Construct
gacattgtgctgacccaatctccagcttctttggctgtgtctctagggcagagggccaccatctcctgcaaggccagccaaagtgttgattatgatggtgatagttatatggattggtaccaacagaaaccaggacagccacccagactcctcatctatcttgtatccaacctagaatctgggatcccagccaggtttagtggcagtgggtctgggacagacttcaccctcaacatccatcctgtggaggaggaggatgctgcaacttattactgtcagcaaactcatgaggatccgtggacgttcggtggaggcaccaagctggaaatcaaa

SEQ ID NO: 231 - DNA, 333 bp, Artificial Sequence; identical to SEQ ID NO: 227
  Synthetic Construct

SEQ ID NO: 232 - DNA, 363 bp, Artificial Sequence
  Synthetic Construct
cagatccagttggtgcagtctggacctgaactgaagaagcctggagagacagtcaagatctcctgcaaggcttctgggtttaccttcacaaactatggaatgaactgggtgaaacaggctccaggaaagggtttaaagtggatgggctggataaacaccttcactggagagccaacacttgctgatgacttcatgggacggtttgccttgtctttggaaacctctgccagcactgcctatttgcagatcaattacctcaaacatgaggacacggctacatatttctgtgcaagaggagggatgggggtacgactccggtacttcgatgtctggggcgcagggaccacggtcaccgtctcctca

SEQ ID NO: 233 - DNA, 354 bp, Artificial Sequence
  Synthetic Construct
gaggtccagctgcagcagtctggacctgagctggagaagcctggcgcttcagtgaagatatcctgcaaggcttctggttactcattcagtgactacaacatgaactgggtgaagcagggcaatggagagagccttgagtggattggatatgttgatccttactatggtgatactaggtacaaccagaacttcaagggcaaggccacattgactgtagacaaatcctccagcacagcctacatgcagctcaagagcctgacatctgaggactctgcagtctattactgtgcactaagtgagacacctcgcgctatggactactggggtcaaggaacctcagtcaccgtctcccca

SEQ ID NO: 234 - DNA, 321 bp, Artificial Sequence
  Synthetic Construct
gacattgtgatgactcagtctccagccaccctgtctgtgactccaggagatagagtctctctttcctgcagggccagccagagtattagcgactacttacactggtatcaacaaaaatcaaatgagtctccaaggcttctcatcaaatatgcttcccaatccatctctgggatcccctccaggttcagtggcagtggatcagggtcagatttcactctcaatatcaacagtgtagaacctgaagatgttggagtgtattactgtcaaaatggtcacagccttccgctcacgttcggtgctgggaccaagctggagctgaaa

SEQ ID NO: 235 - protein, 17 aa, Artificial Sequence
  Synthetic Construct
YVDPYYGDTRYNQNFKG

SEQ ID NO: 236 - protein, 9 aa, Artificial Sequence
  Synthetic Construct
SETPRAMDY

SEQ ID NO: 237 - protein, 11 aa, Artificial Sequence
  Synthetic Construct
RASQSISDYLH

SEQ ID NO: 238 - protein, 7 aa, Artificial Sequence
  Synthetic Construct
YASQSIS

SEQ ID NO: 239 - protein, 9 aa, Artificial Sequence
  Synthetic Construct
QNGHSLPLT

(19) United States
(12) Patent Application Publication
(10) Pub. No.: US 2023/0393144 A1
(43) Pub. Date: Dec. 7, 2023

(54) METHODS FOR MITIGATING INTERFERENCE BY THERAPEUTIC ANTI-CD47 ANTIBODIES IN PRE-TRANSFUSION ASSAYS

(71) Applicant: I-MAB BIOPHARMA US LIMITED, Germantown, MD (US)

(72) Inventors: Wei Cao, Shanghai (CN); Zhengyi Wang, Shanghai (CN); Yanni Zhang, Shanghai (CN); Bingshi Guo, Shanghai (CN)

(21) Appl. No.: 18/031,858

(22) PCT Filed: Oct. 14, 2021
(86) PCT No.: PCT/CN2021/123893

§ 371 Date: Apr. 13, 2023

(30) Foreign Application Priority Data
Oct. 14, 2020 (WO) PCT/CN2020/120869

(51) Int. Cl. G01N 33/68; G01N 33/543; G01N 33/569; C07K 16/28; C07K 16/42

(52) U.S. Cl. CPC G01N 33/686; G01N 33/54393; G01N 33/56966; C07K 16/2803; C07K 16/4258; C07K 2317/76; C07K 2317/52; C07K 2317/92; C07K 2317/54

(57) ABSTRACT

Provided herein are methods of mitigating (such as eliminating) interference in a serological assay caused by a therapeutic anti-CD47 antibody. Also provided are anti-idiotypic antibodies for use in such methods. Also provided are methods of transfusing donor blood to a subject who is under treatment with a therapeutic anti-CD47 antibody.

### CROSS REFERENCE TO RELATED APPLICATIONS

This application claims the priority benefit of International Application No. PCT/CN2020/120869, filed Oct. 14, 2020, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 233002000341SEQLIST.TXT, date recorded: Oct. 12, 2021, size: 144,097 bytes).

### FIELD OF THE INVENTION

This invention relates to methods and reagents for use in reducing interference in serological assays by therapeutic anti-CD47 antibodies.

### BACKGROUND OF THE INVENTION

Therapeutic monoclonal antibodies (mAbs) are increasingly integrated in treating hematologic malignancies and solid-tissue malignancies. While these mAbs aim to target the molecules expressed on tumor cells, because the target of the mAb is also expressed on red blood cells (RBCs) and/or platelets, interference with pretransfusion testing may result.

CD47 is an integrin-associated transmembrane protein (IAP) that has high affinity for signal-regulatory protein alpha (SIRPa) expressed on macrophage membrane. CD47 initiates self-recognition and inhibits phagocytosis by macrophages. As many malignant cells that show high expression of CD47 evade phagocytosis, CD47 has gained attention as a target for treatment of hematologic malignancies and solid tumors. Briefly, therapeutic anti-CD47 antibodies bind CD47 expressed on malignant cells, thereby allowing for phagocytosis and prompting the destruction of malignant cells. However, CD47 is expressed on virtually all tissues and cell types, including RBCs and platelets. Thus, therapeutic anti-CD47 antibodies may also bind and promote macrophage phagocytosis of normal (i.e., non-disease) cells. As a result, anemia and thrombocytopenia may be observed in patients receiving treatment with a therapeutic anti-CD47 antibody. Transfusions are required as an important part of the supportive care of patients who receive such treatments (Transfusion Medicine. 2020; 1-4. DOI: 10(dot)1111/tme(dot)12664).

As noted above, CD47 is expressed on RBCs and platelets. CD47 is also part of the Rh complex. Briefly, the expression of CD47 is affected by Rh phenotype, rr (dce/dce) having the highest expressions, compared to D-positive, and Rhnull having the least. Thus, residual therapeutic anti-CD47 antibody present in patients' serum or plasma may bind to RBCs and/or platelets and cause interference in all phases of pretransfusion testing. Plasma containing anti-CD47 antibody may also affect detection of antibodies to HLA Class I and platelet antigens, as well as interfere with platelet crossmatch testing. Such interference can delay or prevent blood banks from providing cross-match-compatible red blood cells to patients in need. For example, Hu5F9-G4 is a therapeutic anti-CD47 IgG4 antibody that is currently being evaluated in clinical trials for use in the treatment of hematologic or solid malignancies. The interference by Hu5F9-G4 in pretransfusion has been studied, and it has been found that that Hu5F9-G4 interferes with all phases of pretransfusion testing, including ABO reverse typing (TRANSFUSION 2019; 59; 730-737; Transfusion Medicine. 2020; 1-4).

Many previous attempts to remove or mitigate the interference caused by therapeutic anti-CD47 antibodies in serological assays have not been successful. For example, researchers have found that CD47 is not cleaved from test RBCs by treatment with papain, ficin, trypsin, α-chymotrypsin, 0.2M DTT or W.A.R.M. reagent. Currently, the preferred approach to mitigate Hu5F9-G4 interference is to perform multiple alloadsorptions of the plasma with papain-treated allogeneic RBCs or with pooled platelets. This generally requires four adsorptions. For indirect antiglobulin testing (IAT), use of Immucor monoclonal Gamma-clone anti-human globulin (AHG) anti-IgG, which does not detect IgG4 subclass antibodies, could avoid interference, although weak reactivity may be observed due to carryover agglutination (TRANSFUSION 2019; 59; 730-737). More extensive testing beyond standard methods can lead to delays in providing RBCs for transfusion.

International Application No. PCT/US2017/057535, published as WO 2018075857, provides novel anti-CD47 antibodies or immunologically active fragments thereof that have low immunogenicity in humans and cause low or no level of red blood cell depletion or hemagglutination. In addition, these anti-CD47 antibodies have exhibited potent anti-tumor activities. Although therapeutic anti-CD47 antibodies show promise for improved outcomes, patients who receive treatment with such antibodies or present challenges for pretransfusion testing.

Given the predicted increase in the use of therapeutic anti-CD47 antibodies in the treatment of hematological malignancies and solid tumor, there is a need the art for methods of mitigating or eliminating the interference caused by therapeutic anti-CD47 antibodies in serological tests, such as pretransfusion assays.

### SUMMARY OF THE INVENTION

Provided is a method of reducing interference of a therapeutic anti-CD47 antibody in a serological assay using a blood sample from a subject under treatment of the therapeutic anti-CD47 antibody, comprising: adding an anti-idiotypic antibody specifically recognizing an antigen binding portion of the therapeutic anti-CD47 antibody to the blood sample before conducting the serological assay, wherein the therapeutic anti-CD47 antibody competes for human CD47 binding against an anti-CD47 antibody that comprises an HCDR1, an HCDR2 and an HCDR3 as set forth in a light chain variable domain (V_{H}) comprising SEQ ID NO:79 and an LCDR1, an LCDR2 and an LCDR3 as set forth in a light chain variable domain (V_{L}) comprising SEQ ID NO: 80.

Provided is a method of conducting a serological assay using a blood sample of an individual who is under treatment with a therapeutic anti-CD47 antibody, the method comprising: (a) adding an anti-idiotypic antibody specifically recognizing an antigen binding portion of the therapeutic anti-CD47 antibody to the blood sample; and (b) performing the serological assay on the blood sample, wherein the therapeutic anti-CD47 antibody competes for human CD47 binding against an anti-CD47 antibody that comprises an HCDR1, an HCDR2 and an HCDR3 as set forth in a heavy chain variable domain (V_{H}) comprising SEQ ID NO:79 and an LCDR1, an LCDR2 and an LCDR3 as set forth a light chain variable domain (V_{L}) comprising SEQ ID NO:80, and wherein the addition of the anti-idiotypic antibody reduces interference of the therapeutic anti-CD47 antibody in the serological assay.

In some embodiments, the therapeutic anti-CD47 antibody comprises: a V_{H} that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83); and a V_{L} that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} that comprises SEQ ID NO: 79 and a V_{L} that comprises SEQ ID NO: 80. In some embodiments, therapeutic anti-CD47 antibody comprises a human IgG4 Fc region or a variant thereof that comprises an S228P substitution, wherein amino acid numbering is according to the EU index.

In some embodiments, the anti-idiotypic antibody comprises: (a) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106); (b) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109); (c) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising AASNLES (SEQ ID NO: 105); and an LCDR3 comprising QQTHEDPWT (SEQ ID NO: 220); (d) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising QQTHEDPWT (SEQ ID NO: 220); (e) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising AASNLES (SEQ ID NO: 105); and an LCDR3 comprising QQTHEDPWT (SEQ ID NO: 220); (f) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising LVSNLES (SEQ ID NO: 108); and an LCDR3 comprising QQTHEDPWT (SEQ ID NO: 220); (g) a V_{H} domain that comprises an HCDR1 comprising DYNMN (SEQ ID NO: 100); an HCDR2 comprising YVDPYYGDTRYNQNFKG (SEQ ID NO: 235); and an HCDR3 comprising SETPRAMDY (SEQ ID NO: 236); and a light chain variable (V_{L}) domain that comprises an LCDR1 comprising RASQSISDYLH (SEQ ID NO: 237); an LCDR2 comprising YASQSIS (SEQ ID NO: 238); and an LCDR3 comprising QNGHSLPLT (SEQ ID NO: 239). In some embodiments, the anti-idiotypic antibody comprises (a) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106; or (b) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109). In some embodiments, the anti-idiotypic antibody comprises: (a) a V_{H} comprising SEQ ID NO: 110 and a V_{L} comprising SEQ ID NO: 111; (b) a V_{H} comprising SEQ ID NO: 110 and a V_{L} comprising SEQ ID NO: 112; (c) a V_{H} comprising SEQ ID NO: 110 and a V_{L} comprising SEQ ID NO: 113; (d) a V_{H} comprising SEQ ID NO: 110 and a V_{L} comprising SEQ ID NO: 114; (e) a V_{H} comprising SEQ ID NO: 95 and a V_{L} comprising SEQ ID NO: 87; (f) a V_{H} comprising SEQ ID NO: 95 and a V_{L} comprising SEQ ID NO: 88; (g) a V_{H} comprising SEQ ID NO: 95 and a V_{L} comprising SEQ ID NO: 89; (h) a V_{H} comprising SEQ ID NO: 95 and a V_{L} comprising SEQ ID NO: 90; (i) a V_{H} comprising SEQ ID NO: 95 and a V_{L} comprising SEQ ID NO: 91; (j) a V_{H} comprising SEQ ID NO: 95 and a V_{L} comprising SEQ ID NO: 92; or (k) a V_{H} comprising SEQ ID NO: 93 and a V_{L} comprising SEQ ID NO: 94. In some embodiments, the anti-idiotypic antibody comprises: (a) a V_{H} comprising SEQ ID NO: 110 and a V_{L} comprising SEQ ID NO: 111; (b) a V_{H} comprising SEQ ID NO: 110 and a V_{L} comprising SEQ ID NO: 112; (c) a V_{H} comprising SEQ ID NO: 110 and a V_{L} comprising SEQ ID NO: 113; or (d) a V_{H} comprising SEQ ID NO: 110 and a V_{L} comprising SEQ ID NO: 114.

In some embodiments, the binding affinity of the anti-idiotypic antibody to the therapeutic anti-CD47 antibody is higher than the binding affinity of human CD47 on the surface of red blood cells to the therapeutic anti-CD47 antibody. In some embodiments, anti-idiotypic antibody is added to the blood sample in an excess amount relative to the amount of the therapeutic anti-CD47 antibody in the blood sample. In some embodiments, the anti-idiotypic antibody is added to the blood sample in amount sufficient to achieve a molar ratio of between about 1:1 and about 3:1 of anti-idiotypic antibody relative to therapeutic anti-CD47 antibody in the blood sample. In some embodiments, the anti-idiotypic antibody is added to the blood sample in an amount sufficient to achieve a molar ratio of about 2:1 of anti-idiotypic antibody relative to therapeutic anti-CD47 antibody in the blood sample. In some embodiments, the serological assay is selected from the group consisting of direct antiglobulin test (DAT), indirect antiglobulin test (IAT), ABO test, Rh(D) blood typing test, blood cross matching, and coombs test. In some embodiments, the serological assay is an indirect antiglobulin test (IAT). In some embodiments, the serological assay is a direct antiglobulin test (DAT). In some embodiments, the serological assay is an eluate test performed after a DAT assay. In some embodiments, the concentration of the therapeutic anti-CD47 antibody in the blood sample is between about 20 μg/ml and about 1500 μg/ml. In some embodiments, the method comprises incubating the blood sample and the anti-idiotypic antibody for at least about 15 minutes prior to conducting the serological assay. In some embodiments, the incubation is carried out at 37° C.

Provided is a method of conducting a serological assay in a blood sample of an individual who under treatment with a therapeutic anti-CD47 antibody comprising an IgG4 Fc domain, the method comprising: conducting a direct antiglobulin testing (DAT) or indirect antiglobulin testing (IAT) on the blood sample using an anti-human globulin (AHG) anti-IgG that does not recognize a human IgG4 antibody Fc region. In some embodiments, the therapeutic anti-CD47 antibody comprises: (a) a V_{H} domain that comprises an HCDR1 comprising SYAMS (SEQ ID NO: 115); an HCDR2 comprising AISGSGGSTYYADSVKG (SEQ ID NO: 116); and an HCDR3 comprising YSIGRHTFDH (SEQ ID NO: 117) and a (V_{L}) domain that comprises an LCDR1 comprising TRSSGGIASNFVQ (SEQ ID NO: 118); an LCDR2 comprising RDNQRPS (SEQ ID NO: 119); and an LCDR3 comprising QSYDDHNHWV (SEQ ID NO: 120); (b) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (c) a V_{H} domain that comprises an HCDR1 comprising GYAMT (SEQ ID NO: 124); an HCDR2 comprising AITSTGGRTYYADSVKG (SEQ ID NO: 125); and an HCDR3 comprising ESNFRAFDI (SEQ ID NO: 126) and a (V_{L}) domain that comprises an LCDR1 comprising RSSQSLLHSNGYNYLD (SEQ ID NO: 127); an LCDR2 comprising LNSNRAS (SEQ ID NO: 128); and an LCDR3 comprising MQALQIPPT (SEQ ID NO: 129); (d) a V_{H} domain that comprises an HCDR1 comprising DAWMT (SEQ ID NO: 130); an HCDR2 comprising VIYSGGSTYYADSVKG (SEQ ID NO: 131); and an HCDR3 comprising GARGHPGQDY (SEQ ID NO: 132) and a (V_{L}) domain that comprises an LCDR1 comprising TRSSGTIASNFVQ (SEQ ID NO: 133); an LCDR2 comprising ENDRRPS (SEQ ID NO: 134); and an LCDR3 comprising QSYDSSTHGWV (SEQ ID NO: 135); (e) a V_{H} domain that comprises an HCDR1 comprising DYYMS (SEQ ID NO: 136); an HCDR2 comprising YTSRFGSDTNYADSVKG (SEQ ID NO: 137); and an HCDR3 comprising DVHNRDAY (SEQ ID NO: 138) and a (V_{L}) domain that comprises an LCDR1 comprising SGSSSNIGGNSVS (SEQ ID NO: 139); an LCDR2 comprising RNHQRPS (SEQ ID NO: 140); and an LCDR3 comprising ATWDFSLSGFV (SEQ ID NO: 141); (f) a V_{H} domain that comprises an HCDR1 comprising SYAMS (SEQ ID NO: 142); an HCDR2 comprising AISGSGGSTYYADSVKG (SEQ ID NO: 143); and an HCDR3 comprising ADY (SEQ ID NO: 144) and a (V_{L}) domain that comprises an LCDR1 comprising RASQDIRNDLD (SEQ ID NO: 145); an LCDR2 comprising AASNLQS (SEQ ID NO: 146); and an LCDR3 comprising QQSYITPPWT (SEQ ID NO: 147); (g) a V_{H} domain that comprises an HCDR1 comprising SYGMS (SEQ ID NO: 148); an HCDR2 comprising TISGSGSSTNYADSVKG (SEQ ID NO: 149); and an HCDR3 comprising GRYYYDSLDAFDI (SEQ ID NO: 150) and a (V_{L}) domain that comprises an LCDR1 comprising RASQEIRTAYLA (SEQ ID NO: 151); an LCDR2 comprising YASSRAT (SEQ ID NO: 152); and an LCDR3 comprising QQYDTSPPT (SEQ ID NO: 153); (h) a V_{H} domain that comprises an HCDR1 comprising SYAMS (SEQ ID NO: 115); an HCDR2 comprising AISGTGGSTYYADSVKG (SEQ ID NO: 154); and an HCDR3 comprising DKWSSWPTYYFDY (SEQ ID NO: 155) and a (V_{L}) domain that comprises an LCDR1 comprising TRSSGSIASNYVQ (SEQ ID NO: 156); an LCDR2 comprising EDNQRPS (SEQ ID NO: 157); and an LCDR3 comprising QSYDSSNVI (SEQ ID NO: 158); (i) a V_{H} domain that comprises an HCDR1 comprising SYSMA (SEQ ID NO: 159); an HCDR2 comprising AVSNSGVETYYADSVKG (SEQ ID NO: 160); and an HCDR3 comprising RTRQLLTPREFDY (SEQ ID NO: 161) and a (V_{L}) domain that comprises an LCDR1 comprising RASQDITRWLA (SEQ ID NO: 162); an LCDR2 comprising DASSLQS (SEQ ID NO: 163); and an LCDR3 comprising QQGSSVPFT (SEQ ID NO: 164); (j) a V_{H} domain that comprises an HCDR1 comprising NYAMS (SEQ ID NO: 165); an HCDR2 comprising SVSSAGGSTYYADSVKG (SEQ ID NO: 166); and an HCDR3 comprising RVNRAFDL (SEQ ID NO: 167) and a (V_{L}) domain that comprises an LCDR1 comprising RASQSVSSSYLA (SEQ ID NO: 168); an LCDR2 comprising GASSRAT (SEQ ID NO: 169); and an LCDR3 comprising QQYGSSPPMYT (SEQ ID NO: 170); (k) a V_{H} domain that comprises an HCDR1 comprising NAWMS (SEQ ID NO: 171); an HCDR2 comprising RIKSKTDGGTTDYAAPVKG (SEQ ID NO: 172); and an HCDR3 comprising DKSYGYTFDY (SEQ ID NO: 173) and a (V_{L}) domain that comprises an LCDR1 comprising SGSGSNIGSNSVH (SEQ ID NO: 174); an LCDR2 comprising TNNQRPS (SEQ ID NO: 175); and an LCDR3 comprising ATWDDRLSGPV (SEQ ID NO: 176); (1) a V_{H} domain that comprises an HCDR1 comprising SYWMH (SEQ ID NO: 177); an HCDR2 comprising AISGSGAGTYYPDSVKG (SEQ ID NO: 178); and an HCDR3 comprising DRSLSFGFDI (SEQ ID NO: 179) and a (V_{L}) domain that comprises an LCDR1 comprising TRSSGSIGSTYVQ (SEQ ID NO: 180); an LCDR2 comprising KDDQRPS (SEQ ID NO: 181); and an LCDR3 comprising QSSDTSNLV (SEQ ID NO: 182); (m) a V_{H} domain that comprises an HCDR1 comprising RYWMS (SEQ ID NO: 183); an HCDR2 comprising NIKGDGSQTYYADSVKG (SEQ ID NO: 184); and an HCDR3 comprising GAAYHINSWLDP (SEQ ID NO: 185) and a (V_{L}) domain that comprises an LCDR1 comprising RASQSISGNYLA (SEQ ID NO: 186); an LCDR2 comprising GAFRRAT (SEQ ID NO: 187); and an LCDR3 comprising QHYNNFPHT (SEQ ID NO: 188); (n) a V_{H} domain that comprises an HCDR1 comprising HAWMN (SEQ ID NO: 189); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYQVNNRNYLA (SEQ ID NO: 190); an LCDR2 comprising QASTRAS (SEQ ID NO: 85); and an LCDR3 comprising QQYYTPPLA (SEQ ID NO: 86); (o) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQTVLYPLNNRNYLA (SEQ ID NO: 97); an LCDR2 comprising QASTRAS (SEQ ID NO: 85); and an LCDR3 comprising QQYYTPPLA (SEQ ID NO: 86); (p) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86); (q) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86); (r) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSSDI (SEQ ID NO: 192) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (s) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSSDI (SEQ ID NO: 193) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (t) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSSDI (SEQ ID NO: 194) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (u) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDA (SEQ ID NO: 195) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (v) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDS (SEQ ID NO: 196) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (w) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SDRASDK (SEQ ID NO: 98) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (x) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDT (SEQ ID NO: 197) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (y) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSQDI (SEQ ID NO: 198) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (z) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSQDI (SEQ ID NO: 199)) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (aa) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSQDI (SEQ ID NO: 200) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (bb) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86); (cc) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLRPPLN (SEQ ID NO: 202); (dd) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLTPPLN (SEQ ID NO: 99); (ee) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLS (SEQ ID NO: 203); (ff) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLA (SEQ ID NO: 204); (gg) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLNAPLH (SEQ ID NO: 205); (hh) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLEAPLV (SEQ ID NO: 206); (ii) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLH (SEQ ID NO: 207); (jj) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QRLIAPPFT (SEQ ID NO: 208); (kk) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLA (SEQ ID NO: 209); (11) a V_{H} domain that comprises an HCDR1 comprising SYYMH (SEQ ID NO: 210); an HCDR2 comprising EINPNNARINFNEKFKT (SEQ ID NO: 211); and an HCDR3 comprising GYYRYGAWFGY (SEQ ID NO: 212) and a (V_{L}) domain that comprises an LCDR1 comprising RASQDISDYLN (SEQ ID NO: 213); an LCDR2 comprising YISRLHS (SEQ ID NO: 214); and an LCDR3 comprising QQGHTLPWT (SEQ ID NO: 215); or (mm) V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86).

In some embodiments, the therapeutic anti-CD47 antibody comprises: (a) a V_{H} that comprises SEQ ID NO: 1 and a V_{L} that comprises SEQ ID NO: 2; (b) a V_{H} that comprises SEQ ID NO: 3 and a V_{L} that comprises SEQ ID NO: 4; (c) a V_{H} that comprises SEQ ID NO: 5 and a V_{L} that comprises SEQ ID NO: 6; (d) a V_{H} that comprises SEQ ID NO: 7 and a V_{L} that comprises SEQ ID NO: 8; (e) a V_{H} that comprises SEQ ID NO: 9 and a V_{L} that comprises SEQ ID NO: 10; (f) a V_{H} that comprises SEQ ID NO: 11 and a V_{L} that comprises SEQ ID NO: 12; (g) a V_{H} that comprises SEQ ID NO: 13 and a V_{L} that comprises SEQ ID NO: 14; (h) a V_{H} that comprises SEQ ID NO: 14 and a V_{L} that comprises SEQ ID NO: 15; (i) a V_{H} that comprises SEQ ID NO: 16 and a V_{L} that comprises SEQ ID NO: 17; (j) a V_{H} that comprises SEQ ID NO: 18 and a V_{L} that comprises SEQ ID NO: 19; (k) a V_{H} that comprises SEQ ID NO: 20 and a V_{L} that comprises SEQ ID NO: 21; (1) a V_{H} that comprises SEQ ID NO: 22 and a V_{L} that comprises SEQ ID NO: 23; (m) a V_{H} that comprises SEQ ID NO: 23 and a V_{L} that comprises SEQ ID NO: 24; (n) a V_{H} that comprises SEQ ID NO: 25 and a V_{L} that comprises SEQ ID NO: 26; (o) a V_{H} that comprises SEQ ID NO: 27 and a V_{L} that comprises SEQ ID NO: 28; (p) a V_{H} that comprises SEQ ID NO: 29 and a V_{L} that comprises SEQ ID NO: 30; (q) a V_{H} that comprises SEQ ID NO: 31 and a V_{L} that comprises SEQ ID NO: 32; (r) a V_{H} that comprises SEQ ID NO: 33 and a V_{L} that comprises SEQ ID NO: 34; (s) a V_{H} that comprises SEQ ID NO: 35 and a V_{L} that comprises SEQ ID NO: 36; (t) a V_{H} that comprises SEQ ID NO: 37 and a V_{L} that comprises SEQ ID NO: 38; (u) a V_{H} that comprises SEQ ID NO: 39 and a V_{L} that comprises SEQ ID NO: 40; (v) a V_{H} that comprises SEQ ID NO: 41 and a V_{L} that comprises SEQ ID NO: 42; (w) a V_{H} that comprises SEQ ID NO: 43 and a V_{L} that comprises SEQ ID NO: 44; (x) a V_{H} that comprises SEQ ID NO: 45 and a V_{L} that comprises SEQ ID NO: 46; (y) a V_{H} that comprises SEQ ID NO: 47 and a V_{L} that comprises SEQ ID NO: 48; z) a V_{H} that comprises SEQ ID NO: 49 and a V_{L} that comprises SEQ ID NO: 50; (aa) a V_{H} that comprises SEQ ID NO: 51 and a V_{L} that comprises SEQ ID NO: 52; (bb) a V_{H} that comprises SEQ ID NO: 53 and a V_{L} that comprises SEQ ID NO: 54; (cc) a V_{H} that comprises SEQ ID NO: 55 and a V_{L} that comprises SEQ ID NO: 56; (dd) a V_{H} that comprises SEQ ID NO: 57 and a V_{L} that comprises SEQ ID NO: 58; (ee) a V_{H} that comprises SEQ ID NO: 59 and a V_{L} that comprises SEQ ID NO: 60; (ff) a V_{H} that comprises SEQ ID NO: 61 and a V_{L} that comprises SEQ ID NO: 62; (gg) a V_{H} that comprises SEQ ID NO: 63 and a V_{L} that comprises SEQ ID NO: 64; (hh) a V_{H} that comprises SEQ ID NO: 65 and a V_{L} that comprises SEQ ID NO: 66; (ii) a V_{H} that comprises SEQ ID NO: 67 and a V_{L} that comprises SEQ ID NO: 68; (jj) a V_{H} that comprises SEQ ID NO: 69 and a V_{L} that comprises SEQ ID NO: 70; (kk) a V_{H} that comprises SEQ ID NO: 71 and a V_{L} that comprises SEQ ID NO: 72; (ll) a V_{H} that comprises SEQ ID NO: 73 and a V_{L} that comprises SEQ ID NO: 74; (mm) a V_{H} that comprises SEQ ID NO: 75 and a V_{L} that comprises SEQ ID NO: 76; (nn) a V_{H} that comprises SEQ ID NO: 77 and a V_{L} that comprises SEQ ID NO: 78; or (oo) a V_{H} that comprises SEQ ID NO: 79 and a V_{L} that comprises SEQ ID NO: 80. In some embodiments, the therapeutic anti-CD47 antibody comprises a V_{H} that comprises SEQ ID NO: 79 and a V_{L} that comprises SEQ ID NO: 80.

In some embodiments of any of the methods described herein, the therapeutic anti-CD47 antibody comprises a heavy chain comprising the amino acid sequence of SEQ ID NO: 216 or 217 and a light chain comprising the amino acid sequence of SEQ ID NO: 218. In some embodiments, the subject is diagnosed with cancer. In some embodiments, the cancer is a hematological malignancy. In some embodiments, the cancer is solid tumor. In some embodiments, the method comprises a step of adding an enhancer to the blood sample prior to performing the serological assay. In some embodiments, the enhancer is selected from the group consisting of: low ionic strength saline (LISS), polyethylene glycol (PEG), saline, and albumin. In some embodiments, the enhancer is LISS. In some embodiments, the blood sample is selected from the group consisting of: a non-hemolyzed blood sample, a plasma sample, clotted blood, and serum. In some embodiments, the blood sample is a plasma sample. In some embodiments, the method further comprises a step of treating the blood sample with EDTA prior to performing the serological assay.

Also provided herein is a method of transfusing donor blood to a subject who is under treatment with an anti-CD47 antibody, the method comprising: (a) conducting a method described herein on a blood sample from the subject; and (b) transfusing the donor blood into the individual, wherein the donor blood is determined to be compatible with the individual according to a method described herein. In some embodiments, the individual has anemia. In some embodiments, the anemia is induced by the anti-CD47 antibody administered to the individual. In some embodiments, the blood transfusion is carried out within about 3 days after the administration of the anti-CD47 antibody. In some embodiments, the transfusing step is carried out within about 96 hours after the serological assay.

Provided herein is an anti-idiotypic antibody or immunologically active fragment thereof that specifically recognizes the antigen binding portion of a therapeutic anti-CD47 antibody, wherein the anti-idiotypic antibody comprises: (a) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106); (b) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109); (c) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising AASNLES (SEQ ID NO: 105); and an LCDR3 comprising QQTHEDPWT (SEQ ID NO: 220); (d) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising QQTHEDPWT (SEQ ID NO: 220); (e) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising AASNLES (SEQ ID NO: 105); and an LCDR3 comprising QQTHEDPWT (SEQ ID NO: 220); (f) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising LVSNLES (SEQ ID NO: 108); and an LCDR3 comprising QQTHEDPWT (SEQ ID NO: 220); or (g) a V_{H} domain that comprises an HCDR1 comprising DYNMN (SEQ ID NO: 100); an HCDR2 comprising YVDPYYGDTRYNQNFKG (SEQ ID NO: 235); and an HCDR3 comprising SETPRAMDY (SEQ ID NO: 236); and a light chain variable (V_{L}) domain that comprises an LCDR1 comprising RASQSISDYLH (SEQ ID NO: 237); an LCDR2 comprising YASQSIS (SEQ ID NO: 238); and an LCDR3 comprising QNGHSLPLT (SEQ ID NO: 239). In some embodiments, the anti-idiotypic antibody or immunologically active fragment thereof comprises: (a) a V_{H} comprising SEQ ID NO: 110 and a V_{L} comprising SEQ ID NO: 111; (b) a V_{H} comprising SEQ ID NO: 110 and a V_{L} comprising SEQ ID NO: 112; (c) a V_{H} comprising SEQ ID NO: 110 and a V_{L} comprising SEQ ID NO: 113; (d) a V_{H} comprising SEQ ID NO: 110 and a V_{L} comprising SEQ ID NO: 114; (e) a V_{H} comprising SEQ ID NO: 95 and a V_{L} comprising SEQ ID NO: 87; (f) a V_{H} comprising SEQ ID NO: 95 and a V_{L} comprising SEQ ID NO: 88; (g) a V_{H} comprising SEQ ID NO: 95 and a V_{L} comprising SEQ ID NO: 89; (h) a V_{H} comprising SEQ ID NO: 95 and a V_{L} comprising SEQ ID NO: 90; (i) a V_{H} comprising SEQ ID NO: 95 and a V_{L} comprising SEQ ID NO: 91; (j) a V_{H} comprising SEQ ID NO: 95 and a V_{L} comprising SEQ ID NO: 92; or (k) a V_{H} comprising SEQ ID NO: 93 and a V_{L} comprising SEQ ID NO: 94.

Also provided herein is a method of detecting the presence of an anti-CD47 antibody or immunologically active fragment thereof in a sample of an individual, comprising: (a) contacting the sample with an anti-idiotypic antibody described herein, and (b) detecting a complex comprising the anti-idiotypic antibody and the anti-CD47 antibody or fragment thereof, thereby detecting the presence of anti-CD47 antibody or fragment thereof.

In some embodiments, the isolated antibody or immunologically active fragment binds to anti-CD47 antibody or immunologically active fragment thereof. For brevity, the CD47-binding isolated monoclonal antibodies and their immunologically active fragments are referred to hereinafter as “CD47 antibodies”. As well known to a person skilled in the art, CD47 antibodies are interchangeably called “anti-CD47” or “anti-CD47 antibodies”. In some embodiments, the isolated antibody or immunologically active fragment that binds to anti-CD47 antibody is an anti-idiotype antibody.

As used herein, the term “isolated” preceding anti-idiotype antibody of this invention means that the antibody is substantially free of other cellular material. In one embodiment, an isolated antibody is substantially free of other proteins from the same species. In another embodiment, an isolated antibody is expressed by a cell from a different species and is substantially free of other proteins from the different species. A protein may be rendered substantially free of naturally associated components (or components associated with the cellular expression system used to produce the antibody) by isolation, using protein purification techniques well known in the art. In some embodiment, the antibodies or immunologically active fragment of the invention are isolated.

The terms “anti-CD47 antibody”, “anti-CD47”, “CD47 antibody” or “an antibody that binds to CD47” refers to an antibody that is capable of binding CD47 with sufficient affinity such that the antibody is useful as a diagnostic and/or therapeutic agent in targeting CD47.

In some embodiments, the CD47 antibody is a fusion protein, further comprising a therapeutic agent or a marker, and the therapeutic agent or marker is conjugated with the monoclonal antibody.

In some embodiments, the isolated antibody that binds to a CD47 antibody is further labeled with an enzyme, fluorophore, chromophore or light refractive particle.

In some embodiments, a method described herein comprises (such as further comprises) indirect antiglobulin testing (IAT), ABO, Rh(D) blood typing test, blood cross matching, and coombs test.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows the amino acid sequences of CDRs of anti-idiotypic antibody 9F9H11F8.

FIG. 2 shows the amino acid sequences of V_{H} and V_{L} of the anti-idiotypic antibody 9F9H11F8.

FIGS. 3A and 3B show the results of flow-cytometry-based competitive binding assays. FIG. 3A is shows the relative amount of anti-CD47 antibody bound to CD47 expressed on the surface of red blood cells (RBCs) in the presence of increasing concentrations of anti-idiotypic antibody. The fluorescence intensity of 200 μg/ml anti-CD47 antibody bound to RBC, wherein the binding of the anti-CD47 antibody to RBC was detected by labeled secondary antibody, was used as the maximum fluorescence intensity. The fluorescence intensity of RBC itself (i.e., without any anti-CD47 antibody) was used as baseline. Briefly, if the anti-CD47 antibody were bound by the anti-idiotypic antibody, the anti-CD47 antibody would be blocked from binding CD47 on the surfaces of RBCs and would not be detected by flow cytometry analysis. Results of the assay demonstrate that the fluorescence intensity of RBCs decreases gradually as the concentration of anti-idiotypic antibody increases. At a 1:2 ratio of anti-CD47 antibody to anti-idiotypic antibody, the binding of the anti-CD47 antibody to RBCs is completely inhibited (e.g., RBC fluorescence is at baseline levels).

FIG. 3B shows the % inhibition of the binding of ant-CD47 to CD47 expressed on the surface of RBCs at various ratios of anti-CD47 antibody: anti-idiotypic antibody.

FIG. 4 provides the V_{H} and V_{L} sequences of CD47 antibodies that can be bound by anti-idiotypic antibodies described herein.

FIG. 5A shows the V_{H} and V_{L} sequences of anti-idiotypic antibody 37F8C4G12, and FIG. 5B shows the V_{H} and V_{L} sequences of anti-idiotypic antibody 34D8H11B5.

FIG. 6A shows that interference in a serological assay was observed when blood samples containing anti-CD47 were used in the assay.

### DETAILED DESCRIPTION OF THE INVENTION

### Definitions

Before describing the embodiments in detail, it is to be understood that the present disclosure is not limited to particular compositions or biological systems, which can, of course, vary. It is also to be understood that the terminology used herein is for the purpose of describing particular embodiments only, and is not intended to be limiting.

Unless defined otherwise, technical and scientific terms used herein have the same meaning as commonly understood by one of ordinary skill in the art to which this invention belongs.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

The term “CD47” (which is also known as Integrin Associated Protein (IAP), Antigenic Surface Determinant Protein OA3, OA3, CD47 Antigen, Rh-Related Antigen, Integrin-Associated Signal Transducer, Antigen Identified By Monoclonal Antibody 1D8, CD47 glycoprotein) preferably refers to human CD47 and, in particular, to a protein comprising the amino acid sequence

(SEQ ID NO: 96)
MWPLVAALLL GSACCGSAQL LFNKTKSVEF TFCNDTVVIP
CFVTNMEAQN TTEVYVKWKF KGRDIYTFDG ALNKSTVPTD
FSSAKIEVSQ LLKGDASLKM DKSDAVSHTG NYTCEVTELT
REGETIIELK YRVVSWFSPN ENILIVIFPI FAILLFWGQF
GIKTLKYRSG GMDEKTIALL VAGLVITVIV IVGAILFVPG
EYSLKNATGL GLIVISTGIL ILLHYYVFST AIGLTSFVIA
ILVIQVIAYI LAVVGLSLCI AACIPMHGPL LISGLSILAL
AQLLGLVYMK FVASNQKTIQ PPRKAVEEPL NAFKESKGMM NDE

or a variant of said amino acid sequence. The term “CD47” also refers to any post translationally modified variants and conformation variants.

As used herein, the term “antibody” is used in the broadest sense and specifically covers monoclonal antibodies (including full length monoclonal antibodies), polyclonal antibodies, multi-specific antibodies (e.g., bispecific antibodies), and antibody fragments so long as they exhibit the desired biological activity. “Antibodies” (or “Abs”) and “immunoglobulins” (or “Igs”) are glycoproteins having the same structural characteristics. While antibodies exhibit binding specificity to a specific antigen, immunoglobulins include both antibodies and other antibody-like molecules which lack antigen specificity.

As used herein, the term “variable” or “variable region” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (See, e.g., Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. See, e.g., Pluckthun, in The Pharmacology of Monoclonal Antibodies, Vol. 113, Rosenburg and Moore eds., Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxy terminus of the heavy chain CHi domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

There are five major classes of immunoglobulins: IgA, IgD, IgE, IgG, and IgM, and several of these can be further divided into subclasses (isotypes), e.g., IgG1, lgG2, lgG3, lgG4, IgA1, lgA2. The heavy-chain constant domains that correspond to the different classes of immunoglobulins are called α, δ, ϵ, γ, and μ, respectively. The subunit structures and three-dimensional configurations of different classes of immunoglobulins are well known.

As used herein, the term “antibody fragment”, and all grammatical variants thereof, are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. In an antibody fragment comprising one or more heavy chains, the heavy chain(s) can contain any constant domain sequence (e.g. CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

Unless specifically indicated to the contrary, the term “conjugate” used herein is defined as a heterogeneous molecule formed by the covalent attachment of one or more antibody fragment(s) to one or more polymer molecule(s), wherein the heterogeneous molecule is water soluble, i.e. soluble in physiological fluids such as blood, and wherein the heterogeneous molecule is free of any structured aggregate. In the context of the foregoing definition, the term “structured aggregate” refers to (1) any aggregate of molecules in aqueous solution having a spheroid or spheroid shell structure, such that the heterogeneous molecule is not in a micelle or other emulsion structure, and is not anchored to a lipid bilayer, vesicle or liposome; and (2) any aggregate of molecules in solid or insolubilized form, such as a chromatography bead matrix, that does not release the heterogeneous molecule into solution upon contact with an aqueous phase. Accordingly, the term “conjugate” as defined herein encompasses the aforementioned heterogeneous molecule in a precipitate, sediment, bioerodible matrix or other solid capable of releasing the heterogeneous molecule into aqueous solution upon hydration of the solid.

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. Isolated antibody includes the antibody in situ within recombinant cells since at least one component of the antibody's natural environment will not be present. Ordinarily, however, isolated antibody will be prepared by at least one purification step.

As used herein, the term “treatment” or “treating” or any grammatical variation refers to both therapeutic treatment and prophylactic or preventative measures of a disease (such as cancer or a fibrotic disease). Those in need of treatment include those already with the disease as well as those in which the disease is to be prevented. Examples of cancer include, but are not limited to, ovarian cancer, colon cancer, breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, and solid tumors. The fibrotic disease can be, e.g., myocardial infarction, angina, osteoarthritis, pulmonary fibrosis, asthma, cystic fibrosis, bronchitis, or asthma.

As used herein, the term “idiotype” refers to the specific region of an immunoglobulin which imparts its idiotypic character. Immunoglobulin molecules possess variable regions that are responsible for their specific antigen recognition. The features that distinguish one immunoglobulin variable region from another are collectively termed the antibody “idiotype,” which is derived from the Greek for “private form”. In the next step in classification and nomenclature, the variable region idiotypes contain and are defined by a plurality of determinants, termed “idiotope.” The binding site of the antigen, which is thereby recognized by the antibody, is termed the “epitope” and the binding site on the antibody is termed the “paratope”. A paratope may serve as an idiotope, i.e., the paratope may stimulate an anti-idiotypic response in which, like the original epitope, the anti-idiotype antibodies bind to the paratope.

An “anti-idiotypic antibody”, “anti-idiotype antibody” or “anti-ID antibody”, as used herein, is an antibody that binds to the V_{H} and/or V_{L} domain of a therapeutic antibody, in this case the therapeutic antibody is CD47 antibody. Typically, such anti-idiotypic antibodies are prepared by immunizing a mammal such as a mouse with the antibody of interest and producing a hybridoma library and selecting from the panel of antibodies derived from the hybridomas those antibodies that give a clean signal in the assay, whether for the capture reagent or the detectable antibody. In some embodiments, the antibody of the invention is an anti-idiotype antibody, which can be used to monitor the presence or activity of therapeutic anti-CD47 antibody, and used to mitigate the interference of therapeutic anti-CD47 antibody in a pre-transfusion test.

As used herein, the term “biological sample” refers to any biological substance that may contain an antibody of interest. A sample can be biological fluid, such as whole blood or whole blood components including red blood cells, white blood cells, platelets, serum and plasma, ascites, vitreous fluid, lymph fluid, synovial fluid, follicular fluid, seminal fluid, amniotic fluid, milk, saliva, sputum, tears, perspiration, mucus, cerebrospinal fluid, and other constituents of the body that may contain the antibody of interest. In some embodiments, the sample is a body sample from any animal. In some embodiments, the biological sample is from clinical patients or patients treated with a therapeutic anti-CD47 antibody.

As used herein, the term “subject” for purposes of treatment refers to any animal classified as a mammal, including humans, domestic and farm animals, and zoo, sports, or pet animals, such as dogs, horses, cats, cows, etc. Preferably, the mammal is human.

All references cited herein, including patent applications and publications, are hereby incorporated by reference in their entirety.

### DETAILED DESCRIPTION OF THE INVENTION

### Overview

CD47 is a transmembrane protein that interacts with several molecules expressed on the surface of immune cells, including signal regulatory protein alpha (SIRPα). Upon binding CD47, SIRPα initiates a signaling cascade that inhibits phagocytosis and prevents phagocytic removal of healthy cells by the immune system. However, many cancers overexpress CD47 and evade phagocytic clearance. Accordingly, drugs that target CD47 (such as therapeutic anti-CD47 antibodies) are of significant therapeutic interest. CD47 is also expressed on the surface of human red blood cells (RBCs) and platelets. Thus, following the administration of therapeutic anti-CD47 antibody to a subject in need thereof, the therapeutic antibody present in the subject's plasma or bound to the subject's RBCs and/or platelets may cause interference in routine pre-transfusion serological assays. The methods described herein mitigate (or, in some embodiments, eliminate) such interference in pre-transfusion serological assays.

### Methods of Using an Anti-Idiotypic Antibody to Mitigate Interference in a Pre-Transfusion Serological Assay Caused by a Therapeutic Anti-CD47 Antibody

Provided herein are methods of reducing interference of a therapeutic anti-CD47 antibody in a serological assay using a blood sample from a subject under treatment of the therapeutic anti-CD47 antibody that comprise adding an anti-idiotypic antibody that specifically recognizes an antigen binding portion of the therapeutic anti-CD47 antibody to the blood sample before conducting the serological assay on the blood sample. In some embodiments, the blood sample is a non-hemolyzed blood sample, a plasma sample, a clotted blood sample, or a serum sample. In some embodiments, the method further comprises the step of conducting the serological assay. Also provided are methods of conducting a serological assay using a blood sample (e.g., a non-hemolyzed blood sample, a plasma sample, a clotted blood sample, or a serum sample) from a subject under treatment with a therapeutic anti-CD47 antibody, comprising adding an anti-idiotypic antibody that specifically recognizes an antigen binding portion of the therapeutic anti-CD47 antibody to the subject's blood sample and conducting the serological assay on the blood sample. Further details regarding subjects under treatment with an anti-CD47 antibody are provided below. In some embodiments, the serological assay is a direct antiglobulin test (DAT), an indirect antiglobulin test (IAT), an ABO test, an Rh(D) blood typing test, blood cross matching, and/or a coombs test. Further details about these, and other, serological assays are provided below.

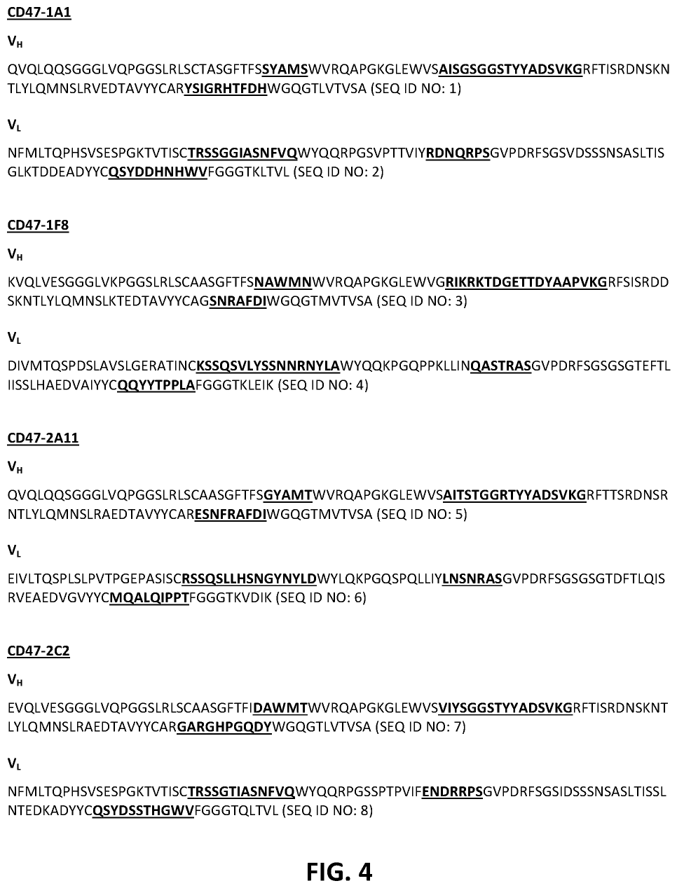

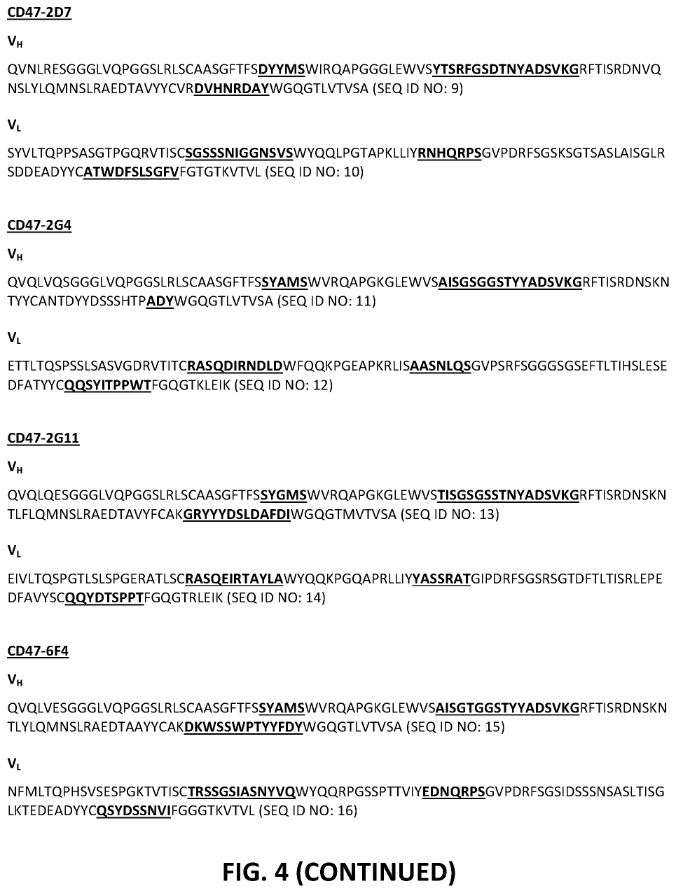

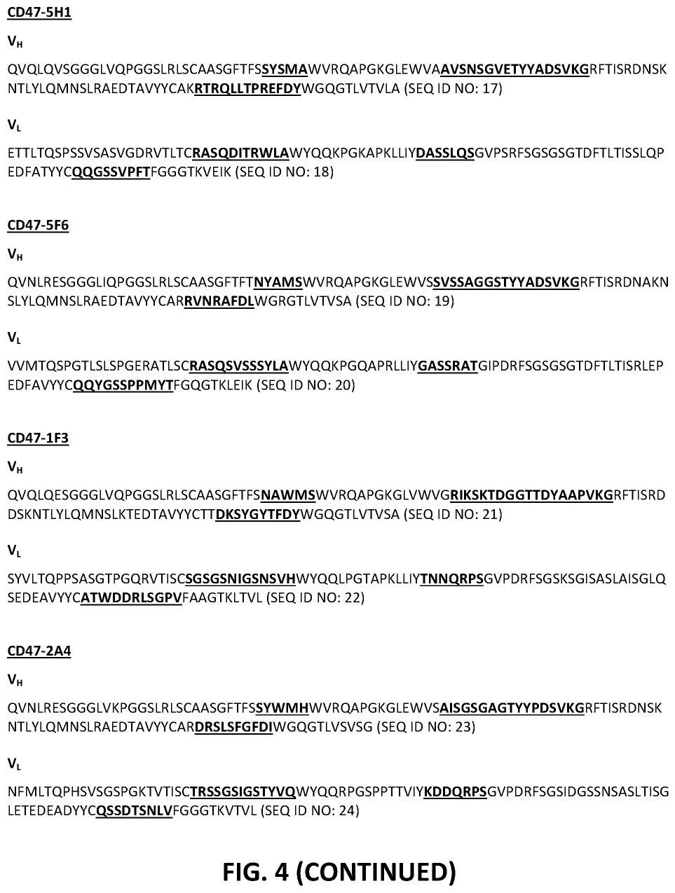

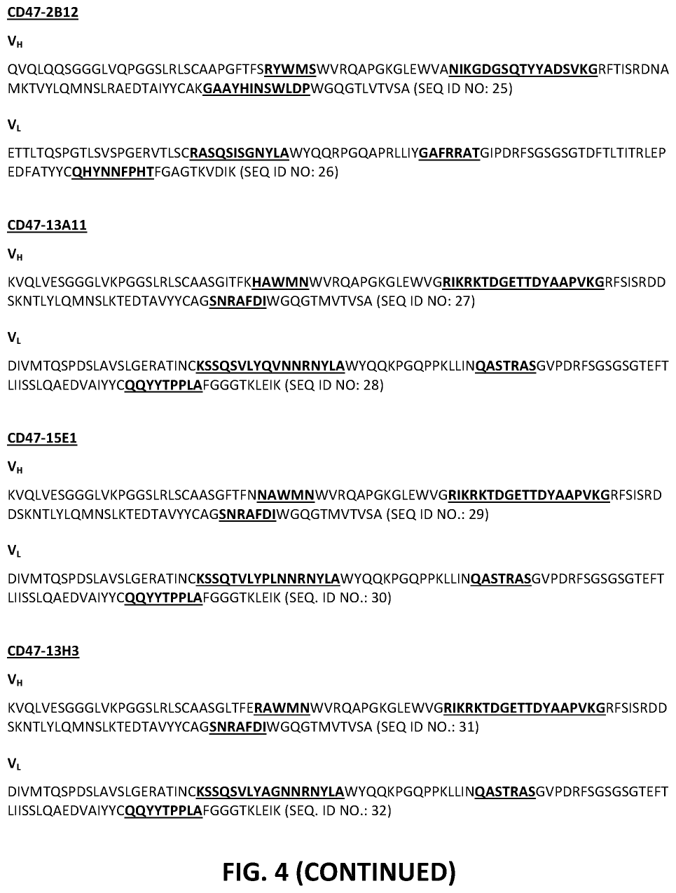

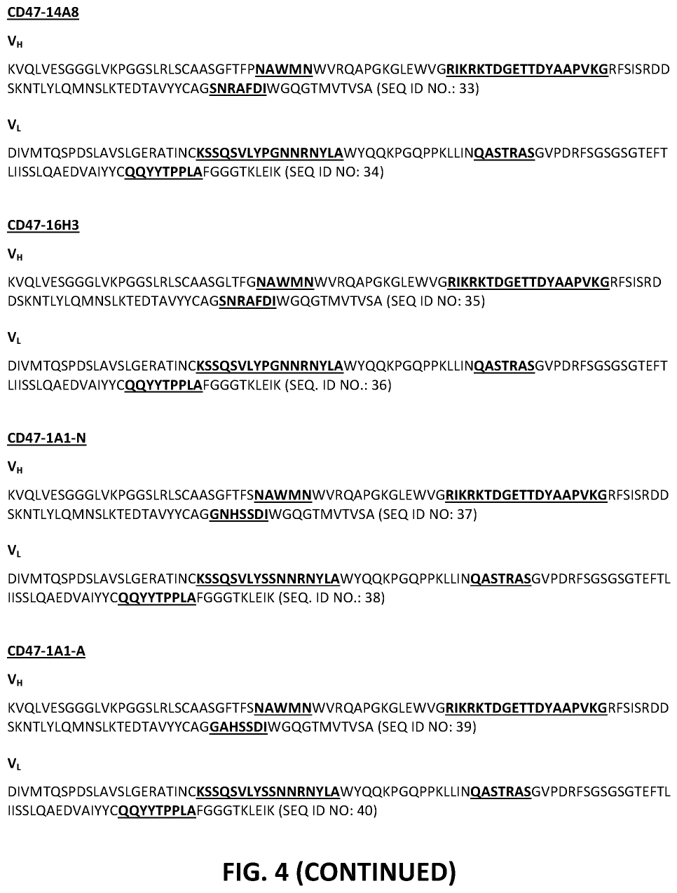

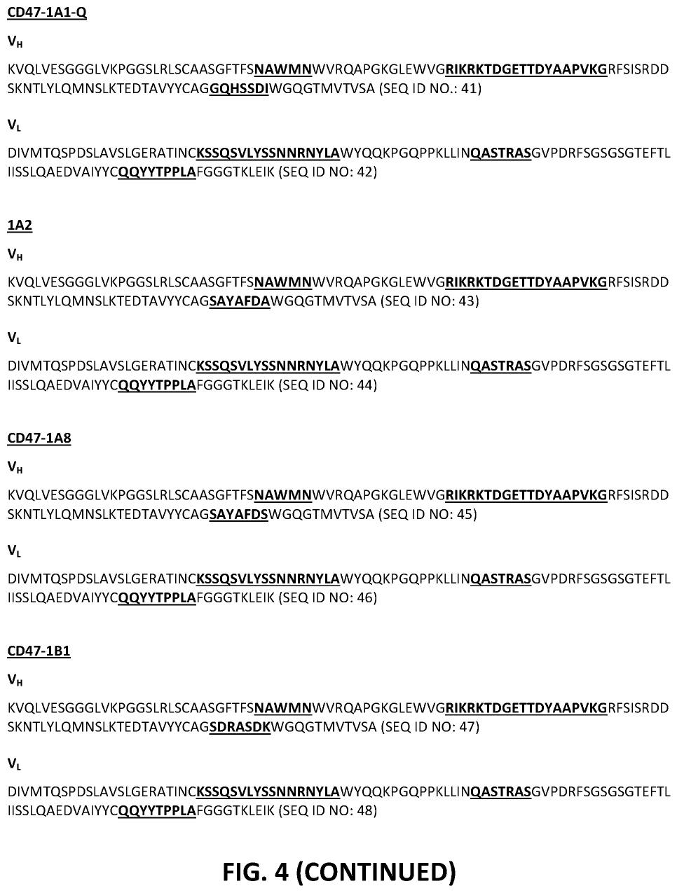

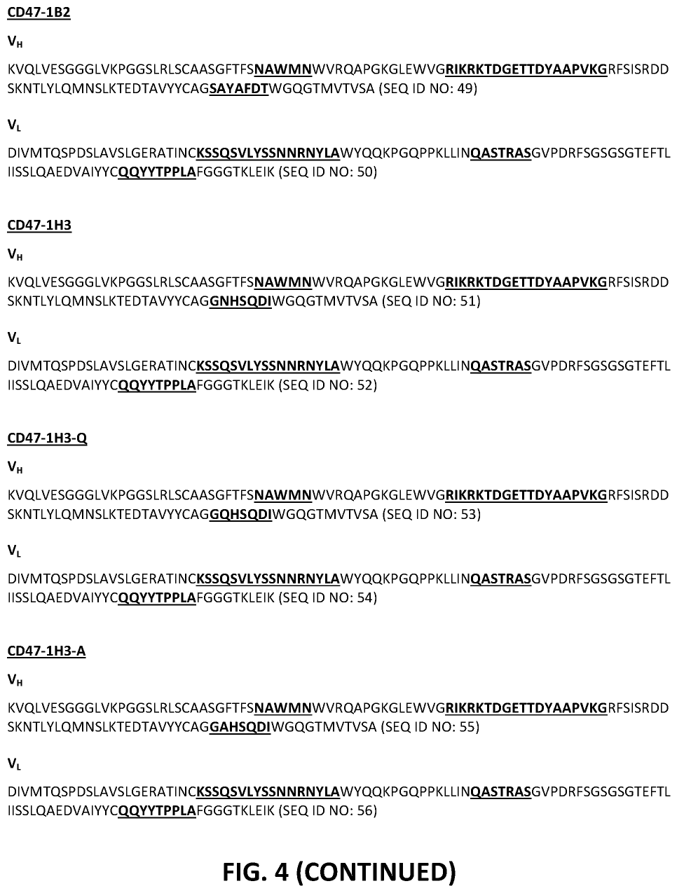

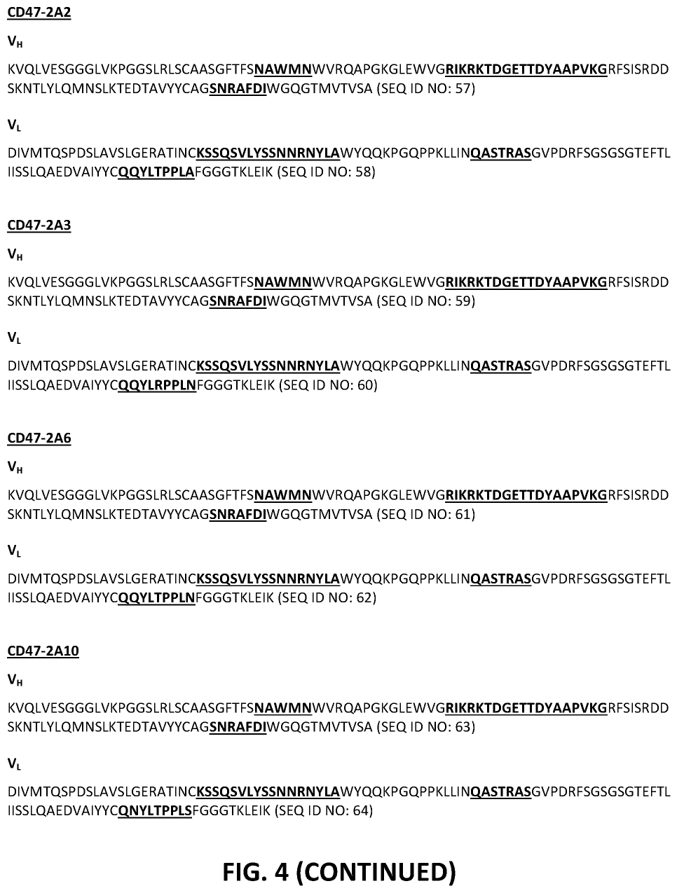

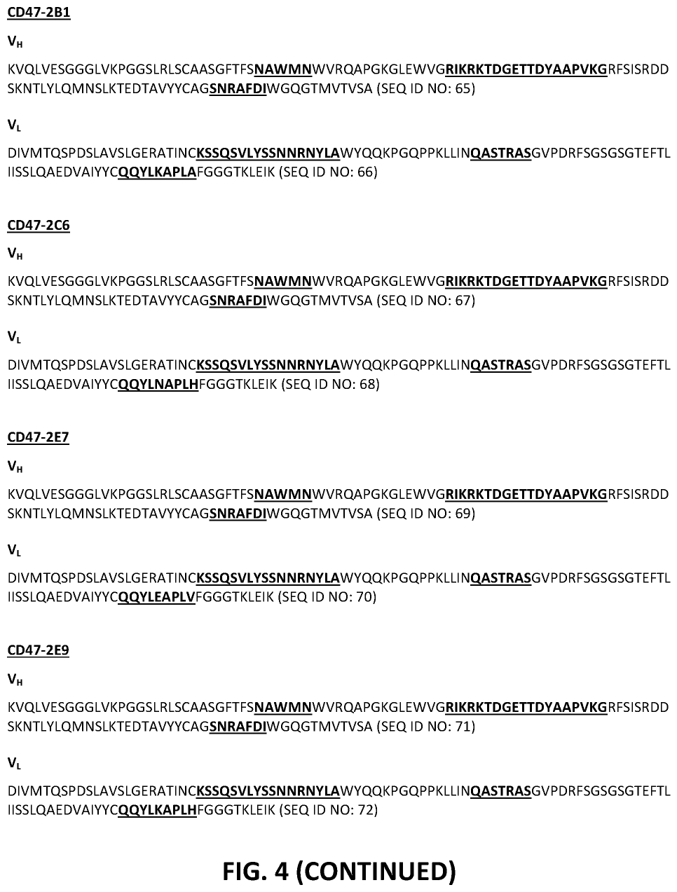

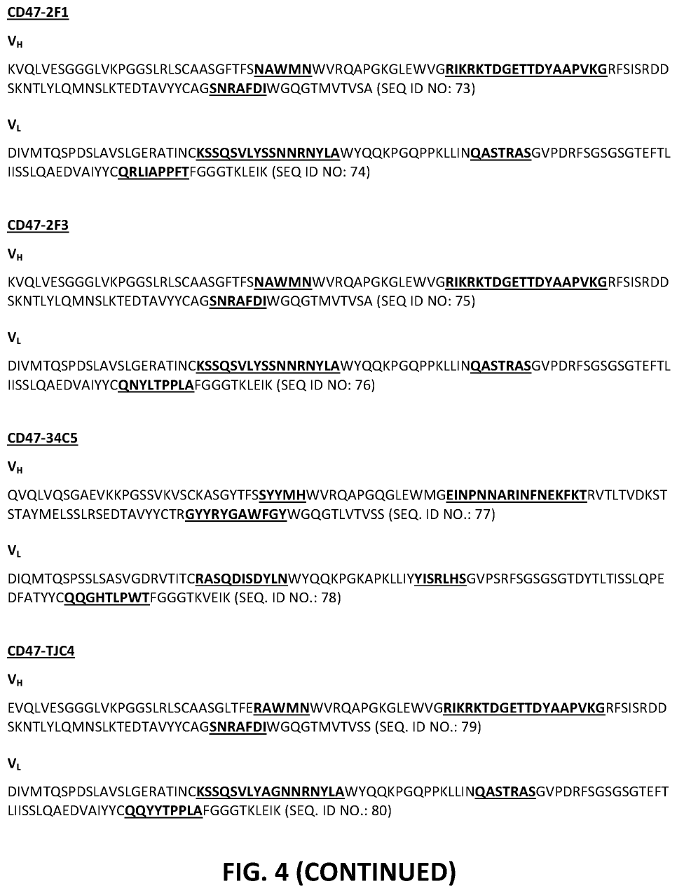

In some embodiments, the therapeutic anti-CD47 antibody competes for human CD47 binding against a second anti-CD47 antibody comprising an HCDR1, an HCDR2 and an HCDR3 as set forth in a heavy chain variable domain (V_{H}) that comprises SEQ ID NO: 1 and an LCDR1, an LCDR2 and an LCDR3 as set forth in a light chain variable domain (V_{L}) that comprises SEQ ID NO: 2. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 3 and an LCDR1, an LCDR2 and an LCDR3 as set forth in a V_{L} that comprises SEQ ID NO: 4. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 5 and an LCDR1, an LCDR2 and an LCDR3 as set forth in a V_{L} that comprises SEQ ID NO: 6. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 7 and an LCDR1, an LCDR2 and an LCDR3 as set forth in a V_{L} that comprises SEQ ID NO: 8. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 9 and an LCDR1, an LCDR2 and an LCDR3 as set forth in a V_{L} that comprises SEQ ID NO: 10. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO:11 and an LCDR1, an LCDR2 and an LCDR3 as set forth in a V_{L} that comprises SEQ ID NO: 12. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 13 and an LCDR1, an LCDR2 and an LCDR3 as set forth in a V_{L} that comprises SEQ ID NO: 14. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO:15 and an LCDR1, an LCDR2 and an LCDR3 as set forth in a V_{L} that comprises SEQ ID NO: 16. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 17 and an LCDR1, an LCDR2 and an LCDR3 as set forth in a V_{L} that comprises SEQ ID NO: 18. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 19 and an LCDR1, an LCDR2 and an LCDR3 as set forth in a V_{L} that comprises SEQ ID NO: 20. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 21 and an LCDR1, an LCDR2 and an LCDR3 as set forth in a V_{L} that comprises SEQ ID NO: 22. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO:23 and an LCDR1, an LCDR2 and an LCDR3 as set forth in a V_{L} that comprises SEQ ID NO: 24. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 25 and an LCDR1, an LCDR2 and an LCDR3 as set forth in a V_{L} that comprises SEQ ID NO: 26. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 27 and an LCDR1, an LCDR2 and an LCDR3 as set forth in a V_{L} that comprises SEQ ID NO: 28. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 29 and an LCDR1, an LCDR2 and an LCDR3 as set forth in a V_{L} that comprises SEQ ID NO: 30. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 31 and an LCDR1, an LCDR2 and an LCDR3 as set forth in a V_{L} that comprises SEQ ID NO: 32. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 33 and an LCDR1, an LCDR2 and an LCDR3 as set forth in a V_{L} that comprises SEQ ID NO: 34. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 35 and an LCDR1, an LCDR2 and an LCDR3 as set forth in a V_{L} that comprises SEQ ID NO: 36. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 37 and an LCDR1, an LCDR2 and an LCDR3 as set forth in a V_{L} that comprises SEQ ID NO: 38. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 39 and an LCDR1, an LCDR2 and an LCDR3 as set forth in a V_{L} that comprises SEQ ID NO: 40. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 41 and an LCDR1, an LCDR2 and an LCDR3 as set forth in a V_{L} that comprises SEQ ID NO: 42. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 43 and an LCDR1, an LCDR2 and an LCDR3 as set forth in a V_{L} that comprises SEQ ID NO: 44. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 45 and an LCDR1, an LCDR2 and an LCDR3 as set forth in a V_{L} that comprises SEQ ID NO: 46. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 47 and an LCDR1, an LCDR2 and an LCDR3 as set forth in a V_{L} that comprises SEQ ID NO: 48. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 49 and an LCDR1, an LCDR2 and an LCDR3 as set forth in a V_{L} that comprises SEQ ID NO: 50. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 51 and an LCDR1, an LCDR2 and an LCDR3 as set forth in a V_{L} that comprises SEQ ID NO: 52. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 53 and an LCDR1, an LCDR2 and an LCDR3 as set forth in a V_{L} that comprises SEQ ID NO: 54. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 55 and an LCDR1, an LCDR2 and an LCDR3 as set forth in a V_{L} that comprises SEQ ID NO: 56. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 57 and an LCDR1, an LCDR2 and an LCDR3 as set forth in a V_{L} that comprises SEQ ID NO: 58. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 59 and an LCDR1, an LCDR2 and an LCDR3 as set forth in a V_{L} that comprises SEQ ID NO: 60. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 61 and an LCDR1, an LCDR2 and an LCDR3 as set forth in a V_{L} that comprises SEQ ID NO: 62. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 63 and an LCDR1, an LCDR2 and an LCDR3 as set forth in a V_{L} that comprises SEQ ID NO: 64. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 65 and an LCDR1, an LCDR2 and an LCDR3 as set forth in a V_{L} that comprises SEQ ID NO: 66. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 67 and an LCDR1, an LCDR2 and an LCDR3 as set forth in a V_{L} that comprises SEQ ID NO: 68. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 69 and an LCDR1, an LCDR2 and an LCDR3 as set forth in a V_{L} that an comprises SEQ ID NO: 70. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 71 and an LCDR1, an LCDR2 and an LCDR3 as set forth in a V_{L} that comprises SEQ ID NO: 72. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 73 and an LCDR1, an LCDR2 and an LCDR3 as set forth in a V_{L} that comprises SEQ ID NO: 74. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 75 and an LCDR1, an LCDR2 and an LCDR3 as set forth in a V_{L} that comprises SEQ ID NO: 76. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 77 and an LCDR1, an LCDR2 and an LCDR3 as set forth in a V_{L} that comprises SEQ ID NO: 78. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 79 and an LCDR1, an LCDR2 and an LCDR3 as set forth in a V_{L} that comprises SEQ ID NO: 80. SEQ ID NOs: 1-80 are shown in FIG. 4. The CDRs of each V_{H} and V_{L} in FIG. 4 are underlined.

In some embodiments, the therapeutic anti-CD47 antibody comprises a V_{H} domain that comprises an HCDR1 comprising SYAMS (SEQ ID NO: 115); an HCDR2 comprising AISGSGGSTYYADSVKG (SEQ ID NO: 116); and an HCDR3 comprising YSIGRHTFDH (SEQ ID NO: 117) and a (V_{L}) domain that comprises an LCDR1 comprising TRSSGGIASNFVQ (SEQ ID NO: 118); an LCDR2 comprising RDNQRPS (SEQ ID NO: 119); and an LCDR3 comprising QSYDDHNHWV (SEQ ID NO: 120). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising GYAMT (SEQ ID NO: 124); an HCDR2 comprising AITSTGGRTYYADSVKG (SEQ ID NO: 125); and an HCDR3 comprising ESNFRAFDI (SEQ ID NO: 126) and a (V_{L}) domain that comprises an LCDR1 comprising RSSQSLLHSNGYNYLD (SEQ ID NO: 127); an LCDR2 comprising LNSNRAS (SEQ ID NO: 128); and an LCDR3 comprising MQALQIPPT (SEQ ID NO: 129 In some embodiments, the therapeutic anti-CD47 antibody comprises a V_{H} domain that comprises an HCDR1 comprising DAWMT (SEQ ID NO: 130); an HCDR2 comprising VIYSGGSTYYADSVKG (SEQ ID NO: 131); and an HCDR3 comprising GARGHPGQDY (SEQ ID NO: 132) and a (V_{L}) domain that comprises an LCDR1 comprising TRSSGTIASNFVQ (SEQ ID NO: 133); an LCDR2 comprising ENDRRPS (SEQ ID NO: 134); and an LCDR3 comprising QSYDSSTHGWV (SEQ ID NO: 135). In some embodiments, the therapeutic anti-CD47 antibody comprises a V_{H} domain that comprises an HCDR1 comprising DYYMS (SEQ ID NO: 136); an HCDR2 comprising YTSRFGSDTNYADSVKG (SEQ ID NO: 137); and an HCDR3 comprising DVHNRDAY (SEQ ID NO: 138) and a (V_{L}) domain that comprises an LCDR1 comprising SGSSSNIGGNSVS (SEQ ID NO: 139); an LCDR2 comprising RNHQRPS (SEQ ID NO: 140); and an LCDR3 comprising ATWDFSLSGFV (SEQ ID NO: 141). In some embodiments, the therapeutic anti-CD47 antibody comprises a V_{H} domain that comprises an HCDR1 comprising SYAMS (SEQ ID NO: 142); an HCDR2 comprising AISGSGGSTYYADSVKG (SEQ ID NO: 143); and an HCDR3 comprising ADY (SEQ ID NO: 144) and a (V_{L}) domain that comprises an LCDR1 comprising RASQDIRNDLD (SEQ ID NO: 145); an LCDR2 comprising AASNLQS (SEQ ID NO: 146); and an LCDR3 comprising QQSYITPPWT (SEQ ID NO: 147). In some embodiments, the therapeutic anti-CD47 antibody comprises a V_{H} domain that comprises an HCDR1 comprising SYGMS (SEQ ID NO: 148); an HCDR2 comprising TISGSGSSTNYADSVKG (SEQ ID NO: 149); and an HCDR3 comprising GRYYYDSLDAFDI (SEQ ID NO: 150) and a (V_{L}) domain that comprises an LCDR1 comprising RASQEIRTAYLA (SEQ ID NO: 151); an LCDR2 comprising YASSRAT (SEQ ID NO: 152); and an LCDR3 comprising QQYDTSPPT (SEQ ID NO: 153). In some embodiments, the therapeutic anti-CD47 antibody comprises a V_{H} domain that comprises an HCDR1 comprising SYAMS (SEQ ID NO: 115); an HCDR2 comprising AISGTGGSTYYADSVKG (SEQ ID NO: 154); and an HCDR3 comprising DKWSSWPTYYFDY (SEQ ID NO: 155) and a (V_{L}) domain that comprises an LCDR1 comprising TRSSGSIASNYVQ (SEQ ID NO: 156); an LCDR2 comprising EDNQRPS (SEQ ID NO: 157); and an LCDR3 comprising QSYDSSNVI (SEQ ID NO: 158). In some embodiments, the therapeutic anti-CD47 antibody comprises a V_{H} domain that comprises an HCDR1 comprising SYSMA (SEQ ID NO: 159); an HCDR2 comprising AVSNSGVETYYADSVKG (SEQ ID NO: 160); and an HCDR3 comprising RTRQLLTPREFDY (SEQ ID NO: 161) and a (V_{L}) domain that comprises an LCDR1 comprising RASQDITRWLA (SEQ ID NO: 162); an LCDR2 comprising DASSLQS (SEQ ID NO: 163); and an LCDR3 comprising QQGSSVPFT (SEQ ID NO: 164). In some embodiments, the therapeutic anti-CD47 antibody comprises a V_{H} domain that comprises an HCDR1 comprising NYAMS (SEQ ID NO: 165); an HCDR2 comprising SVSSAGGSTYYADSVKG (SEQ ID NO: 166); and an HCDR3 comprising RVNRAFDL (SEQ ID NO: 167) and a (V_{L}) domain that comprises an LCDR1 comprising RASQSVSSSYLA (SEQ ID NO: 168); an LCDR2 comprising GASSRAT (SEQ ID NO: 169); and an LCDR3 comprising QQYGSSPPMYT (SEQ ID NO: 170). In some embodiments, the therapeutic anti-CD47 antibody comprises a V_{H} domain that comprises an HCDR1 comprising NAWMS (SEQ ID NO: 171); an HCDR2 comprising RIKSKTDGGTTDYAAPVKG (SEQ ID NO: 172); and an HCDR3 comprising DKSYGYTFDY (SEQ ID NO: 173) and a (V_{L}) domain that comprises an LCDR1 comprising SGSGSNIGSNSVH (SEQ ID NO: 174); an LCDR2 comprising TNNQRPS (SEQ ID NO: 175); and an LCDR3 comprising ATWDDRLSGPV (SEQ ID NO: 176). In some embodiments, the therapeutic anti-CD47 antibody comprises a V_{H} domain that comprises an HCDR1 comprising SYWMH (SEQ ID NO: 177); an HCDR2 comprising AISGSGAGTYYPDSVKG (SEQ ID NO: 178); and an HCDR3 comprising DRSLSFGFDI (SEQ ID NO: 179) and a (V_{L}) domain that comprises an LCDR1 comprising TRSSGSIGSTYVQ (SEQ ID NO: 180); an LCDR2 comprising KDDQRPS (SEQ ID NO: 181); and an LCDR3 comprising QSSDTSNLV (SEQ ID NO: 182). In some embodiments, the therapeutic anti-CD47 antibody comprises a V_{H} domain that comprises an HCDR1 comprising RYWMS (SEQ ID NO: 183); an HCDR2 comprising NIKGDGSQTYYADSVKG (SEQ ID NO: 184); and an HCDR3 comprising GAAYHINSWLDP (SEQ ID NO: 185) and a (V_{L}) domain that comprises an LCDR1 comprising RASQSISGNYLA (SEQ ID NO: 186); an LCDR2 comprising GAFRRAT (SEQ ID NO: 187); and an LCDR3 comprising QHYNNFPHT (SEQ ID NO: 188). In some embodiments, the therapeutic anti-CD47 antibody comprises a V_{H} domain that comprises an HCDR1 comprising HAWMN (SEQ ID NO: 189); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYQVNNRNYLA (SEQ ID NO: 190); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQTVLYPLNNRNYLA (SEQ ID NO: 97); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSSDI (SEQ ID NO: 192) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSSDI (SEQ ID NO: 193) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSSDI (SEQ ID NO: 194) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDA (SEQ ID NO: 195) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDS (SEQ ID NO: 196) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SDRASDK (SEQ ID NO: 98) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDT (SEQ ID NO: 197) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSQDI (SEQ ID NO: 198) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSQDI (SEQ ID NO: 199)) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSQDI (SEQ ID NO: 200) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLRPPLN (SEQ ID NO: 202). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLTPPLN (SEQ ID NO: 99). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLS (SEQ ID NO: 203). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLA (SEQ ID NO: 204). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLNAPLH (SEQ ID NO: 205). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLEAPLV (SEQ ID NO: 206). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLH (SEQ ID NO: 207). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QRLIAPPFT (SEQ ID NO: 208). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLA (SEQ ID NO: 209). In some embodiments, the therapeutic anti-CD47 antibody comprises a V_{H} domain that comprises an HCDR1 comprising SYYMH (SEQ ID NO: 210); an HCDR2 comprising EINPNNARINFNEKFKT (SEQ ID NO: 211); and an HCDR3 comprising GYYRYGAWFGY (SEQ ID NO: 212) and a (V_{L}) domain that comprises an LCDR1 comprising RASQDISDYLN (SEQ ID NO: 213); an LCDR2 comprising YISRLHS (SEQ ID NO: 214); and an LCDR3 comprising QQGHTLPWT (SEQ ID NO: 215). In some embodiments, the therapeutic anti-CD47 antibody comprises a V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the CDRs of the therapeutic anti-CD47 antibody are defined according to the Kabat numbering system (Kabat et al., Sequences of Proteins of Immunological Interest, 5th Ed. Public Health Service, National Institutes of Health, Bethesda, Md. (1991)).

In some embodiments, the therapeutic anti-CD47 antibody comprises a V_{H} that comprises SEQ ID NO: 1 and a V_{L} that comprises SEQ ID NO: 2. In some embodiments, the therapeutic anti-CD47 comprises a V_{H} that comprises SEQ ID NO: 3 and a V_{L} that comprises SEQ ID NO: 4. In some embodiments, the therapeutic anti-CD47 comprises a V_{H} that comprises SEQ ID NO: 5 and a V_{L} that comprises SEQ ID NO: 6. In some embodiments, the therapeutic anti-CD47 antibody comprises a V_{H} that comprises SEQ ID NO: 7 and a V_{L} that comprises SEQ ID NO: 8. In some embodiments, the therapeutic anti-CD47 comprises a V_{H} that comprises SEQ ID NO: 9 and a V_{L} that comprises SEQ ID NO: 10. In some embodiments, the therapeutic anti-CD47 antibody comprises a V_{H} that comprises SEQ ID NO: 11 and a V_{L} that comprises SEQ ID NO: 12. In some embodiments, the therapeutic anti-CD47 antibody comprises a V_{H} that comprises SEQ ID NO: 13 and a V_{L} that comprises SEQ ID NO: 14. In some embodiments, the therapeutic anti-CD47 antibody comprises a V_{H} that comprises SEQ ID NO:15 and a V_{L} that comprises SEQ ID NO: 16. In some embodiments, the therapeutic anti-CD47 antibody comprises V_{H} that comprises SEQ ID NO: 17 and a V_{L} that comprises SEQ ID NO: 18. In some embodiments, the therapeutic anti-CD47 antibody comprises a V_{H} that comprises SEQ ID NO: 19 and a V_{L} that comprises SEQ ID NO: 20. In some embodiments, the therapeutic anti-CD47 antibody comprises a V_{H} that comprises SEQ ID NO: 21 and a V_{L} that comprises SEQ ID NO: 22. In some embodiments, the therapeutic anti-CD47 antibody comprises a V_{H} that comprises SEQ ID NO:23 and a V_{L} that comprises SEQ ID NO: 24. In some embodiments, the therapeutic anti-CD47 antibody comprises in a V_{H} that comprises SEQ ID NO: 25 and a V_{L} that comprises SEQ ID NO: 26. In some embodiments, the therapeutic anti-CD47 antibody comprises a V_{H} that comprises SEQ ID NO: 27 and a V_{L} that comprises SEQ ID NO: 28. In some embodiments, the therapeutic anti-CD47 antibody comprises a V_{H} that comprises SEQ ID NO: 29 and a V_{L} that comprises SEQ ID NO: 30. In some embodiments, the therapeutic anti-CD47 antibody comprises a V_{H} that comprises SEQ ID NO: 31 and a V_{L} that comprises SEQ ID NO: 32. In some embodiments, the therapeutic anti-CD47 antibody a V_{H} that comprises SEQ ID NO: 33 and a V_{L} that comprises SEQ ID NO: 34. In some embodiments, the therapeutic anti-CD47 antibody comprises a V_{H} that comprises SEQ ID NO: 35 and a V_{L} that comprises SEQ ID NO: 36. In some embodiments, the therapeutic anti-CD47 antibody comprises a V_{H} that comprises SEQ ID NO: 37 and a V_{L} that comprises SEQ ID NO: 38. In some embodiments, the therapeutic anti-CD47 antibody comprises a V_{H} that comprises SEQ ID NO: 39 and a V_{L} that comprises SEQ ID NO: 40. In some embodiments, the therapeutic anti-CD47 antibody comprises a V_{H} that comprises SEQ ID NO: 41 and a V_{L} that comprises SEQ ID NO: 42. In some embodiments, the therapeutic anti-CD47 antibody a V_{H} that comprises SEQ ID NO: 43 and a V_{L} that comprises SEQ ID NO: 44. In some embodiments, the therapeutic anti-CD47 antibody comprises a V_{H} that comprises SEQ ID NO: 45 and a V_{L} that comprises SEQ ID NO: 46. In some embodiments, the therapeutic anti-CD47 antibody comprises a V_{H} that comprises SEQ ID NO: 47 and a V_{L} that comprises SEQ ID NO: 48. In some embodiments, the therapeutic anti-CD47 antibody comprises a V_{H} that comprises SEQ ID NO: 49 and a V_{L} that comprises SEQ ID NO: 50. In some embodiments, the therapeutic anti-CD47 antibody comprises a V_{H} that comprises SEQ ID NO: 51 and a V_{L} that comprises SEQ ID NO: 52. In some embodiments, the therapeutic anti-CD47 comprises a V_{H} that comprises SEQ ID NO: 53 and a V_{L} that comprises SEQ ID NO: 54. In some embodiments, the therapeutic anti-CD47 antibody comprises a V_{H} that comprises SEQ ID NO: 55 and a V_{L} that comprises SEQ ID NO: 56. In some embodiments, the therapeutic anti-CD47 antibody comprises a V_{H} that comprises SEQ ID NO: 57 and a V_{L} that comprises SEQ ID NO: 58. In some embodiments, the therapeutic anti-CD47 antibody comprises a V_{H} that comprises SEQ ID NO: 59 and a V_{L} that comprises SEQ ID NO: 60. In some embodiments, the therapeutic anti-CD47 antibody comprises a V_{H} that comprises SEQ ID NO: 61 and a V_{L} that comprises SEQ ID NO: 62. In some embodiments, the therapeutic anti-CD47 antibody comprises a V_{H} that comprises SEQ ID NO: 63 and a V_{L} that comprises SEQ ID NO: 64. In some embodiments, the therapeutic anti-CD47 antibody comprises a V_{H} that comprises SEQ ID NO: 65 and a V_{L} that comprises SEQ ID NO: 66. In some embodiments, the therapeutic anti-CD47 antibody comprises a V_{H} that comprises SEQ ID NO: 67 and a V_{L} that comprises SEQ ID NO: 68. In some embodiments, the therapeutic anti-CD47 antibody comprises a V_{H} that comprises SEQ ID NO: 69 and a V_{L} that an comprises SEQ ID NO: 70. In some embodiments, the therapeutic anti-CD47 antibody comprises a V_{H} that comprises SEQ ID NO: 71 and a V_{L} that comprises SEQ ID NO: 72. In some embodiments, the therapeutic anti-CD47 antibody comprises a V_{H} that comprises SEQ ID NO: 73 and a V_{L} that comprises SEQ ID NO: 74. In some embodiments, the therapeutic anti-CD47 antibody comprises a V_{H} that comprises SEQ ID NO: 75 and a V_{L} that comprises SEQ ID NO: 76. In some embodiments, the therapeutic anti-CD47 antibody comprises a V_{H} that comprises SEQ ID NO: 77 and a V_{L} that comprises SEQ ID NO: 78. In some embodiments, the therapeutic anti-CD47 antibody comprises a V_{H} that comprises SEQ ID NO: 79 a V_{L} that comprises SEQ ID NO: 80. SEQ ID NOs: 1-80 are shown in FIG. 4.

In some embodiments, the therapeutic anti-CD47 antibody comprises a V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the CDRs of the therapeutic anti-CD47 antibody are defined according to the Kabat numbering system. In some embodiments, the therapeutic anti-CD47 comprises an HCDR1, an HCDR2 and an HCDR3 as set forth in a V_{H} that comprises SEQ ID NO: 79 and an LCDR1, an LCDR2 and an LCDR3 as set forth in a V_{L} that comprises SEQ ID NO: 80. In some embodiments, the therapeutic anti-CD47 antibody comprises a V_{H} that comprises SEQ ID NO: 79 a V_{L} that comprises SEQ ID NO: 80. In some embodiments, the therapeutic anti-CD47 antibody is a full-length antibody. In some embodiments, the therapeutic anti-CD47 comprises (such as further comprises) a human IgG4 Fc region or a variant thereof comprising an S228P substitution (wherein amino numbering is according to the EU index). In some embodiments, the therapeutic anti-CD47 comprises a heavy chain comprising the amino acid sequence of SEQ ID NO: 216 and a light chain comprising the amino acid sequence of SEQ ID NO: 218. In some embodiments, the therapeutic anti-CD47 comprises a heavy chain comprising the amino acid sequence of SEQ ID NO: 217 and a light chain comprising the amino acid sequence of SEQ ID NO: 218.

(SEQ ID NO: 216)
EVQLVESGGG LVKPGGSLRL SCAASGLIFE RAWMNWVRQA
PGKGLEWVGR IKRKTDGETT DYAAPVKGRF SISRDDSKNT
LYLQMNSLKT EDTAVYYCAG SNRAFDIWGQ GTMVTVSSAS
TKGPSVFPLA PCSRSTSEST AALGCLVKDY FPEPVTVSWN
SGALTSGVHT FPAVLQSSGL YSLSSVVTVP SSSLGTKTYT
CNVDHKPSNT KVDKRVESKY GPPCPPCPAP EFLGGPSVFL
FPPKPKDTLM ISRTPEVTCV VVDVSQEDPE VQFNWYVDGV
EVHNAKTKPR EEQFNSTYRV VSVLTVLHQD WLNGKEYKCK
VSNKGLPSSI EKTISKAKGQ PREPQVYTLP PSQEEMTKNQ
VSLTCLVKGF YPSDIAVEWE SNGQPENNYK TTPPVLDSDG
SFFLYSRLTV DKSRWQEGNV FSCSVMHEAL HNHYTQKSLS LSLGK
(SEQ ID NO: 217)
EVQLVESGGG LVKPGGSLRL SCAASGLTFE RAWMNWVRQA
PGKGLEWVGR IKRKTDGETT DYAAPVKGRF SISRDDSKNT
LYLQMNSLKT EDTAVYYCAG SNRAFDIWGQ GTMVTVSSAS
TKGPSVFPLA PCSRSTSEST AALGCLVKDY FPEPVTVSWN
SGALTSGVHT FPAVLQSSGL YSLSSVVTVP SSSLGTKTYT
CNVDHKPSNT KVDKRVESKY GPPCPPCPAP EFLGGPSVFL
FPPKPKDTLM ISRTPEVTCV VVDVSQEDPE VQFNWYVDGV
EVHNAKTKPR EEQFNSTYRV VSVLTVLHQD WLNGKEYKCK
VSNKGLPSSI EKTISKAKGQ PREPQVYTLP PSQEEMTKNQ
VSLTCLVKGF YPSDIAVEWE SNGQPENNYK TTPPVLDSDG
SFFLYSRLTV DKSRWQEGNV FSCSVMHEAL HNHYTQKSLS LSLG
(SEQ ID NO: 218)
DIVMTQSPDS LAVSLGERAT INCKSSQSVL YAGNNRNYLA
WYQQKPGQPP KLLINQASTR ASGVPDRFSG SGSGTEFTLI
ISSLQAEDVA IYYCQQYYTP PLAFGGGTKL EIKRTVAAPS
VFIFPPSDEQ LKSGTASVVC LLNNFYPREA KVQWKVDNAL
QSGNSQESVT EQDSKDSTYS LSSTLTLSKA DYEKHKVYAC
EVTHQGLSSP VTKSFNRGEC

Any of the exemplary anti-idiotypic antibodies described in further detail elsewhere herein can be used in these methods, and the embodiments provided below are not intended to be limiting.

In some embodiments, the affinity of the anti-idiotypic antibody to the therapeutic anti-CD47 antibody is higher than the affinity of the therapeutic anti-CD47 antibody for human CD47 (e.g., human CD47 expressed on the surface of RBCs and/or platelets). In some embodiments, the affinity of the anti-idiotypic antibody for the therapeutic anti-CD47 antibody is at least about any one of 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 25-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than the affinity of the therapeutic anti-CD47 antibody for human CD47 (e.g., human CD47 expressed on the surface of RBCs and/or platelets).

In some embodiments, the amount of anti-idiotypic antibody agent added to a subject's blood sample (e.g., a non-hemolyzed blood sample, a plasma sample, a clotted blood sample, or a serum sample) is an amount sufficient to achieve an excess amount of anti-idiotypic antibody relative to the amount of therapeutic anti-CD47 antibody in the subject's blood sample. In some embodiments, the amount of anti-idiotypic antibody is an amount sufficient to bind substantially all (such as all) of the therapeutic anti-CD47 antibody in the subject's blood sample. In some embodiments, the concentration of the therapeutic anti-CD47 antibody in the blood sample is between about 20 μg and about 1500 μg/ml (e.g., about any one of 20, 30, 40, 50, 60, 70, 80, 90, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 μg/ml, including any range in between these values). In some embodiments, the amount of anti-idiotypic antibody agent added to the subject's blood sample (e.g., a non-hemolyzed blood sample, a plasma sample, a clotted blood sample, or a serum sample) is an amount sufficient to achieve any one of about, e.g., a 1-fold, 2-fold, 2.5-fold, 3-fold, 3.5-fold, 4-fold, 4.5-fold, 5-fold, 10-fold, 25-fold, 50-fold, 75-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1500-fold, 2000-fold, 2500-fold, 3000-fold, 3500-fold, 4000-fold, 4500-fold or 5000-fold molar ratio of the anti-idiotypic antibody relative to the amount of therapeutic anti-CD47 antibody in the subject's blood sample (e.g., a non-hemolyzed blood sample, a plasma sample, a clotted blood sample, or a serum sample), including any range in between these values. For example, if the amount of anti-idiotypic antibody agent added to a subject's blood sample is the same as the amount of therapeutic anti-CD47 antibody in the sample, the molar ration of anti-idiotypic antibody to therapeutic anti-CD47 antibody in the sample is a 1-fold ratio, i.e., a 1:1 ratio. Correspondingly, if the amount of anti-idiotypic antibody agent added to the subject's blood sample is twice as the amount of therapeutic anti-CD47 antibody in the sample, the molar ratio of anti-idiotypic antibody to therapeutic anti-CD47 antibody in the sample is a 2-fold ratio, i.e., a 2:1 ratio.

In some embodiments, the amount of the anti-idiotypic antibody added to the subject's blood sample (e.g., a non-hemolyzed blood sample, a plasma sample, a clotted blood sample, or a serum sample) is sufficient to achieve a concentration of any one of about 20 μg/ml, 30 μg/ml, 40 μg/ml, 50 μg/ml, 60 μg/ml, 70 μg/ml, 80 μg/ml, 90 μg/ml, 100 μg/ml, 200 μg/ml, 300 μg/ml, 400 μg/ml, 500 μg/ml, 600 μg/ml, 700 μg/ml, 800 μg/ml, 900 μg/ml, 1 mg/ml, 1.25 mg/ml, 1.5 mg/ml, 1.75 mg/ml, 2 mg/ml, 2.25 mg/ml, 2.5 mg/ml, 2.75 mg/ml, 3 mg/ml, 3.25 mg/ml, 3.5 mg/ml, 3.75 mg/ml, 4 mg/ml 4.25 mg/ml. 4.5 mg/ml, 4.75 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 150 mg/ml, 200 mg/ml, 250 mg/ml, 300 mg/ml, 350 mg/ml, 400 mg/ml, 450 mg/ml, 500 mg/ml, 550 mg/ml, 600 mg/ml, 650 mg/ml, 700 mg/ml, 750 mg/ml, 800 mg/ml, 850 mg/ml, 900 mg/ml, 1000 mg/ml, 1100 mg/ml, 1150 mg/ml, 1200 mg/ml, 1250 mg/ml, 1300 mg/ml, 1350 mg/ml, 1400 mg/ml, 1450 mg/ml, 1500 mg/ml, 1550 mg/ml, 1600 mg/ml, 1650 mg/ml, 1700 mg/ml, 1750 mg/ml, 1800 mg/ml, 1850 mg/ml, 1900 mg/ml, or 2000 mg/ml of the anti-idiotypic antibody, including any range in between these values. In some embodiments, at least about any one of 5 g, 10 μg, 50 μg, 100 μg, 200 μg, 300 μg, 400 μg, 500 μg, 600 μg, 700 μg, 800 μg, 900 μg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg. 4.5 mg, 4.75 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, or 2000 mg of the anti-idiotypic antibody is added to the subject's blood sample (e.g., a non-hemolyzed blood sample, a plasma sample, a clotted blood sample, or a serum sample).

In some embodiments, the anti-idiotypic antibody is added to the subject's blood sample (e.g., a non-hemolyzed blood sample, a plasma sample, a clotted blood sample, or a serum sample) in an amount to achieve at least about any one of a 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold ratio of anti-idiotypic antibody relative to the K_{D} of the therapeutic anti-CD47 antibody for human CD47, including any range in between these values. In some embodiments, anti-idiotypic antibody is added to the subject's blood sample (e.g., a non-hemolyzed blood sample, a plasma sample, a clotted blood sample, or a serum sample) in an amount to achieve at least about any one of a 500-fold, 1000-fold, 5000-fold, 10⁴-fold, 10⁵-fold, 10⁶-fold, 10⁷-fold, 10⁸-fold, 10⁹-fold, or 10¹⁰-fold ratio of anti-idiotypic antibody relative to the K_{D} of the therapeutic anti-CD47 antibody for human CD47, including any range in between these values.

In some embodiments the anti-idiotypic antibody is added to the subject's blood sample (e.g., a non-hemolyzed blood sample, a plasma sample, a clotted blood sample, or a serum sample) in an amount to achieve about any one of a 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold ratio of anti-idiotypic antibody relative to the K_{D} of the anti-idiotypic antibody for the therapeutic anti-CD47 antibody, including any range in between these values. In some embodiments the anti-idiotypic antibody is added to the subject's blood sample (e.g., a non-hemolyzed blood sample, a plasma sample, a clotted blood sample, or a serum sample) in an amount to achieve about any one of a 500-fold, 1000-fold, 5000-fold, 10⁴-fold, 105-fold, 10⁶-fold, 10⁷-fold, 10⁸-fold, 10⁹-fold, or 10¹⁰-fold ratio of anti-idiotypic antibody relative to the K_{D} of the anti-idiotypic antibody for the therapeutic anti-CD47 antibody, including any range in between these values.

In some embodiments, the method comprises using two or more anti-idiotypic antibodies described herein. Thus, in some embodiment two or more anti-idiotypic antibodies (e.g., in any combination) that specifically recognize an antigen binding portion of the therapeutic anti-CD47 antibody to the subject's blood sample (e.g., a non-hemolyzed blood sample, a plasma sample, a clotted blood sample, or a serum sample).

In some embodiments, the method is performed in solution. In some embodiments, method comprises (such as further comprises) incubating the blood sample and the anti-idiotypic antibody for at least about any one of 5, 10, 15, 20, 25, 30, 35, 40, 45, 50, 55, 60, 75, 90. 105, or 120 minutes prior to conducting the serological assay, including any range in between these values. In some embodiments, the incubation is carried out at about 37° C. (such as between e.g., 34° C. and 40° C.).

In some embodiments, the anti-idiotypic antibody is immobilized to a solid phase before the method is performed, e.g., via adsorption to a matrix or surface, covalent coupling, or non-covalent coupling. In some embodiments, the anti-idiotypic antibody is capable of binding the therapeutic anti-CD47 antibody following immobilization of the anti-idiotypic antibody to the solid phase. The solid phase or surface used for immobilization can be any inert support or carrier that is essentially water insoluble and useful in immunoassays, including supports in the form of, for example, surfaces, particles, porous matrices, cellulose polymer sponge (ImmunoCAP®, Phadia), and the like. Examples of commonly used supports include small sheets, Sephadex, polyvinyl chloride, plastic beads, microparticles, assay plates, or test tubes manufactured from polyethylene, polypropylene, polystyrene, and the like. In some embodiments, the anti-idiotypic antibody is coated on a microtiter plate, such as a multi-well microtiter plate that can be used to analyze multiple samples simultaneously.

In some embodiments, the method comprises (such as further comprise) a step of adding an enhancer to the subject's blood sample prior to conducting the method of mitigating interference and/or the serological assay. In some embodiments, the enhancer is added to the blood sample (e.g., a non-hemolyzed blood sample, a plasma sample, a clotted blood sample, or a serum sample) prior to the addition of the anti-idiotypic antibody. Additionally or alternatively, in some embodiments, the enhancer is added to the blood sample (e.g., a non-hemolyzed blood sample, a plasma sample, a clotted blood sample, or a serum sample) after the anti-idiotypic antibody has been added but prior to conducting the serological assay. Additionally or alternatively, in some embodiments, the enhancer is added to the blood sample (e.g., a non-hemolyzed blood sample, a plasma sample, a clotted blood sample, or a serum sample) prior to the agglutination step of the serological assay (e.g., prior to the addition of an agglutination agent, such as anti-human globulin (AHG), to the blood sample). An “enhancer” refers to an agent that enhances agglutination in the serological assay and reduces incubation time, e.g., by promoting blood group antibody-antigen reactions. In some embodiments, the enhancer is low ionic strength saline (LISS) or polyethylene glycol (PEG). In some embodiments, the method comprises treating the blood sample with EDTA.

In some embodiments, the method comprises (such as further comprises) comprises transfusing donor blood to the subject, wherein the donor blood is determined to be compatible with the subject, according to the results of the serological assay. Further details regarding transfusing donor blood to a subject under treatment with a therapeutic anti-CD47 antibody are provided below.

Anti-Idiotypic Antibodies that Recognize an Antigen-Binding Portion of a Therapeutic Anti-CD47 Antibody

Provided herein are novel anti-idiotypic antibodies (and immunologically active fragments thereof) that recognize/bind to an antigen-binding portion of a therapeutic anti-CD47 antibody. As used herein, an “immunologically active fragment” of an antibody refers to an antigen-binding fragment of said antibody. The terms “immunologically active fragment” and “antigen-binding fragment” are used interchangeably herein. Any of the anti-idiotypic antibodies described below can be used with the methods described above.

In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising SYAMS (SEQ ID NO: 115); an HCDR2 comprising AISGSGGSTYYADSVKG (SEQ ID NO: 116); and an HCDR3 comprising YSIGRHTFDH (SEQ ID NO: 117) and a (V_{L}) domain that comprises an LCDR1 comprising TRSSGGIASNFVQ (SEQ ID NO: 118); an LCDR2 comprising RDNQRPS (SEQ ID NO: 119); and an LCDR3 comprising QSYDDHNHWV (SEQ ID NO: 120). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising GYAMT (SEQ ID NO: 124); an HCDR2 comprising AITSTGGRTYYADSVKG (SEQ ID NO: 125); and an HCDR3 comprising ESNFRAFDI (SEQ ID NO: 126) and a (V_{L}) domain that comprises an LCDR1 comprising RSSQSLLHSNGYNYLD (SEQ ID NO: 127); an LCDR2 comprising LNSNRAS (SEQ ID NO: 128); and an LCDR3 comprising MQALQIPPT (SEQ ID NO: 129). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising DAWMT (SEQ ID NO: 130); an HCDR2 comprising VIYSGGSTYYADSVKG (SEQ ID NO: 131); and an HCDR3 comprising GARGHPGQDY (SEQ ID NO: 132) and a (V_{L}) domain that comprises an LCDR1 comprising TRSSGTIASNFVQ (SEQ ID NO: 133); an LCDR2 comprising ENDRRPS (SEQ ID NO: 134); and an LCDR3 comprising QSYDSSTHGWV (SEQ ID NO: 135). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising DYYMS (SEQ ID NO: 136); an HCDR2 comprising YTSRFGSDTNYADSVKG (SEQ ID NO: 137); and an HCDR3 comprising DVHNRDAY (SEQ ID NO: 138) and a (V_{L}) domain that comprises an LCDR1 comprising SGSSSNIGGNSVS (SEQ ID NO: 139); an LCDR2 comprising RNHQRPS (SEQ ID NO: 140); and an LCDR3 comprising ATWDFSLSGFV (SEQ ID NO: 141). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising SYAMS (SEQ ID NO: 142); an HCDR2 comprising AISGSGGSTYYADSVKG (SEQ ID NO: 143); and an HCDR3 comprising ADY (SEQ ID NO: 144) and a (V_{L}) domain that comprises an LCDR1 comprising RASQDIRNDLD (SEQ ID NO: 145); an LCDR2 comprising AASNLQS (SEQ ID NO: 146); and an LCDR3 comprising QQSYITPPWT (SEQ ID NO: 147). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising SYGMS (SEQ ID NO: 148); an HCDR2 comprising TISGSGSSTNYADSVKG (SEQ ID NO: 149); and an HCDR3 comprising GRYYYDSLDAFDI (SEQ ID NO: 150) and a (V_{L}) domain that comprises an LCDR1 comprising RASQEIRTAYLA (SEQ ID NO: 151); an LCDR2 comprising YASSRAT (SEQ ID NO: 152); and an LCDR3 comprising QQYDTSPPT (SEQ ID NO: 153). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising SYAMS (SEQ ID NO: 115); an HCDR2 comprising AISGTGGSTYYADSVKG (SEQ ID NO: 154); and an HCDR3 comprising DKWSSWPTYYFDY (SEQ ID NO: 155) and a (V_{L}) domain that comprises an LCDR1 comprising TRSSGSIASNYVQ (SEQ ID NO: 156); an LCDR2 comprising EDNQRPS (SEQ ID NO: 157); and an LCDR3 comprising QSYDSSNVI (SEQ ID NO: 158). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising SYSMA (SEQ ID NO: 159); an HCDR2 comprising AVSNSGVETYYADSVKG (SEQ ID NO: 160); and an HCDR3 comprising RTRQLLTPREFDY (SEQ ID NO: 161) and a (V_{L}) domain that comprises an LCDR1 comprising RASQDITRWLA (SEQ ID NO: 162); an LCDR2 comprising DASSLQS (SEQ ID NO: 163); and an LCDR3 comprising QQGSSVPFT (SEQ ID NO: 164). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NYAMS (SEQ ID NO: 165); an HCDR2 comprising SVSSAGGSTYYADSVKG (SEQ ID NO: 166); and an HCDR3 comprising RVNRAFDL (SEQ ID NO: 167) and a (V_{L}) domain that comprises an LCDR1 comprising RASQSVSSSYLA (SEQ ID NO: 168); an LCDR2 comprising GASSRAT (SEQ ID NO: 169); and an LCDR3 comprising QQYGSSPPMYT (SEQ ID NO: 170). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMS (SEQ ID NO: 171); an HCDR2 comprising RIKSKTDGGTTDYAAPVKG (SEQ ID NO: 172); and an HCDR3 comprising DKSYGYTFDY (SEQ ID NO: 173) and a (V_{L}) domain that comprises an LCDR1 comprising SGSGSNIGSNSVH (SEQ ID NO: 174); an LCDR2 comprising TNNQRPS (SEQ ID NO: 175); and an LCDR3 comprising ATWDDRLSGPV (SEQ ID NO: 176). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising SYWMH (SEQ ID NO: 177); an HCDR2 comprising AISGSGAGTYYPDSVKG (SEQ ID NO: 178); and an HCDR3 comprising DRSLSFGFDI (SEQ ID NO: 179) and a (V_{L}) domain that comprises an LCDR1 comprising TRSSGSIGSTYVQ (SEQ ID NO: 180); an LCDR2 comprising KDDQRPS (SEQ ID NO: 181); and an LCDR3 comprising QSSDTSNLV (SEQ ID NO: 182). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising RYWMS (SEQ ID NO: 183); an HCDR2 comprising NIKGDGSQTYYADSVKG (SEQ ID NO: 184); and an HCDR3 comprising GAAYHINSWLDP (SEQ ID NO: 185) and a (V_{L}) domain that comprises an LCDR1 comprising RASQSISGNYLA (SEQ ID NO: 186); an LCDR2 comprising GAFRRAT (SEQ ID NO: 187); and an LCDR3 comprising QHYNNFPHT (SEQ ID NO: 188). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising HAWMN (SEQ ID NO: 189); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYQVNNRNYLA (SEQ ID NO: 190); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQTVLYPLNNRNYLA (SEQ ID NO: 97); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSSDI (SEQ ID NO: 192) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSSDI (SEQ ID NO: 193) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSSDI (SEQ ID NO: 194) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDA (SEQ ID NO: 195) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDS (SEQ ID NO: 196) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SDRASDK (SEQ ID NO: 98) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDT (SEQ ID NO: 197) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSQDI (SEQ ID NO: 198) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSQDI (SEQ ID NO: 199)) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSQDI (SEQ ID NO: 200) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLRPPLN (SEQ ID NO: 202). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLTPPLN (SEQ ID NO: 99). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLS (SEQ ID NO: 203). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLA (SEQ ID NO: 204). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLNAPLH (SEQ ID NO: 205). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLEAPLV (SEQ ID NO: 206). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLH (SEQ ID NO: 207). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QRLIAPPFT (SEQ ID NO: 208). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLA (SEQ ID NO: 209). In some embodiments, anti-idiotypic antibody (or the immunologically active fragment thereof) recognizes/binds to the antigen-binding portion of a therapeutic anti-CD47 antibody that comprises a V_{H} domain that comprises an HCDR1 comprising SYYMH (SEQ ID NO: 210); an HCDR2 comprising EINPNNARINFNEKFKT (SEQ ID NO: 211); and an HCDR3 comprising GYYRYGAWFGY (SEQ ID NO: 212) and a (V_{L}) domain that comprises an LCDR1 comprising RASQDISDYLN (SEQ ID NO: 213); an LCDR2 comprising YISRLHS (SEQ ID NO: 214); and an LCDR3 comprising QQGHTLPWT (SEQ ID NO: 215). In some embodiments, the anti-idiotypic antibody (or the immunologically active fragment thereof) recognizes/binds to the antigen-binding portion of a therapeutic anti-CD47 antibody that comprises a V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the CDRs of the therapeutic anti-CD47 antibody are defined according to the Kabat numbering system (Kabat et al., Sequences of Proteins of Immunological Interest, 5th Ed. Public Health Service, National Institutes of Health, Bethesda, Md. (1991)).

In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 1 and a V_{L} that comprises SEQ ID NO: 2. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 3 and a V_{L} that comprises SEQ ID NO: 4. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 5 and a V_{L} that comprises SEQ ID NO: 6. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 7 and a V_{L} that comprises SEQ ID NO: 8. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 9 and a V_{L} that comprises SEQ ID NO: 10. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO:11 and a V_{L} that comprises SEQ ID NO: 12. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 13 and a V_{L} that comprises SEQ ID NO: 14. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO:15 and a V_{L} that comprises SEQ ID NO: 16. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 17 and a V_{L} that comprises SEQ ID NO: 18. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 19 and a V_{L} that comprises SEQ ID NO: 20. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 21 and a V_{L} that comprises SEQ ID NO: 22. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO:23 and a V_{L} that comprises SEQ ID NO: 24. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 25 and a V_{L} that comprises SEQ ID NO: 26. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 27 and a V_{L} that comprises SEQ ID NO: 28. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 29 and a V_{L} that comprises SEQ ID NO: 30. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 31 and a V_{L} that comprises SEQ ID NO: 32. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 33 and a V_{L} that comprises SEQ ID NO: 34. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 35 and a V_{L} that comprises SEQ ID NO: 36. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 37 and a V_{L} that comprises SEQ ID NO: 38. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 39 and a V_{L} that comprises SEQ ID NO: 40. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 41 and a V_{L} that comprises SEQ ID NO: 42. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 43 and a V_{L} that comprises SEQ ID NO: 44. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 45 and a V_{L} that comprises SEQ ID NO: 46. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 47 and a V_{L} that comprises SEQ ID NO: 48. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 49 and a V_{L} that comprises SEQ ID NO: 50. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 51 and a V_{L} that comprises SEQ ID NO: 52. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 53 and a V_{L} that comprises SEQ ID NO: 54. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 55 and a V_{L} that comprises SEQ ID NO: 56. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 57 and a V_{L} that comprises SEQ ID NO: 58. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 59 and a V_{L} that comprises SEQ ID NO: 60. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 61 and a V_{L} that comprises SEQ ID NO: 62. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 63 and a V_{L} that comprises SEQ ID NO: 64. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 65 and a V_{L} that comprises SEQ ID NO: 66. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 67 and a V_{L} that comprises SEQ ID NO: 68. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 69 and a V_{L} that an comprises SEQ ID NO: 70. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 71 and a V_{L} that comprises SEQ ID NO: 72. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 73 and a V_{L} that comprises SEQ ID NO: 74. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 75 and an a V_{L} that comprises SEQ ID O: 76. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 77 and a V_{L} that comprises SEQ ID NO: 78. In some embodiments, the anti-idiotypic antibody (or the immunologically active fragment thereof) recognizes/binds to the antigen-binding portion of a therapeutic anti-CD47 antibody that comprises a V_{H} that comprises SEQ ID NO: 79 and a V_{L} that comprises SEQ ID NO: 80. (See FIG. 4, which provides the amino acid sequences of SEQ ID NOs: 1-80. The CDRs in each V_{H} and V_{L} are underlined.)

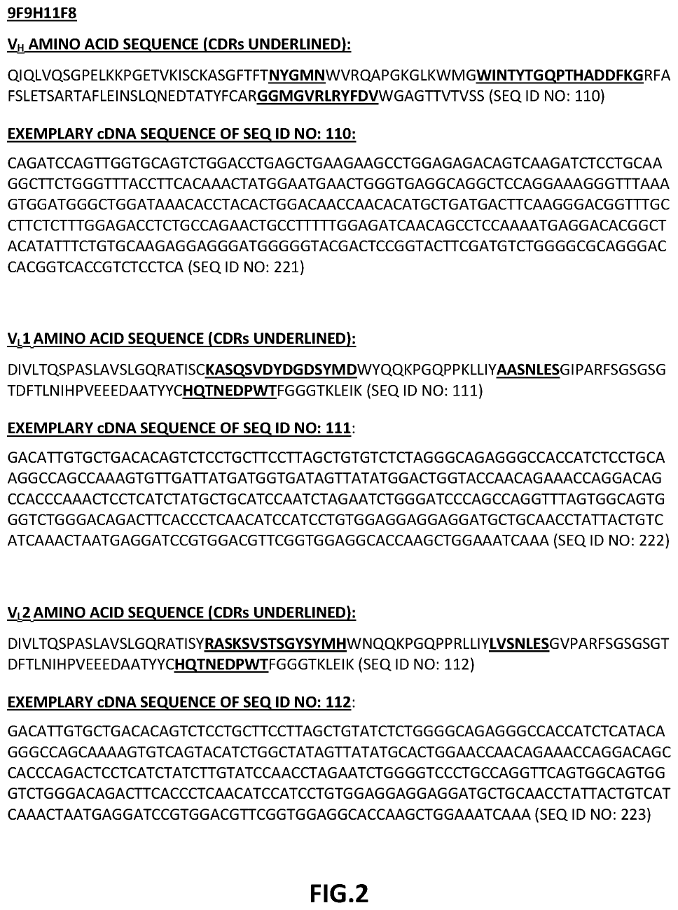

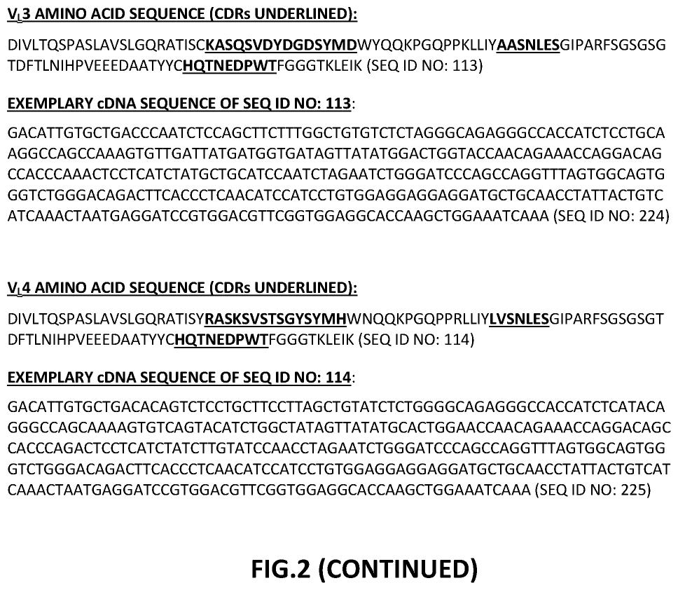

In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106). See, e.g., FIG. 2. In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109). See, e.g., FIG. 2. In some embodiments, the CDRs of the therapeutic anti-CD47 antibody are defined according to the Kabat numbering system (Kabat et al., Sequences of Proteins of Immunological Interest, 5th Ed. Public Health Service, National Institutes of Health, Bethesda, Md. (1991)). In some embodiments, the anti-idiotypic antibody (or immunologically active fragment thereof) comprises a heavy chain variable domain (VH) comprising an amino acid sequence that has at least about 95%, 96%, 97%, 98%, 99%, or 100% sequence identity to an amino acid sequence set forth in SEQ ID NO: 110, and, optionally, a light chain variable domain (VL) comprising an amino acid sequence that has at least about 95%, 96%, 97%, 98%, 99%, or 100% sequence identity to an amino acid sequence set forth in any one of SEQ ID NOs: 111-114. The amino acid sequences of SEQ ID NOs: 110-114 are provided in FIG. 2. SEQ ID NOs: 221-225, which are exemplary cDNA sequences of 110-114, respectively, are also provided in FIG. 2. In some embodiments, the anti-idiotypic antibody (or immunologically active fragment thereof) comprises 3 CDRs of a VH domain comprising SEQ ID NO: 110. Additionally or alternatively, in some embodiments, the anti-idiotypic antibody (or immunologically active fragment thereof) comprises 3 CDRs of a VL domain comprising any one of SEQ ID NOs: 111-114. In some embodiments, the 3 CDRs of the VH domain are CDRs according to Kabat, Chothia, AbM or Contact numbering scheme. Additionally or alternatively, in some embodiments, the 3 CDRs of the VL domain are CDRs according to Kabat, Chothia, AbM or Contact numbering scheme. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO:111. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO:112. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO:113. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO: 114.

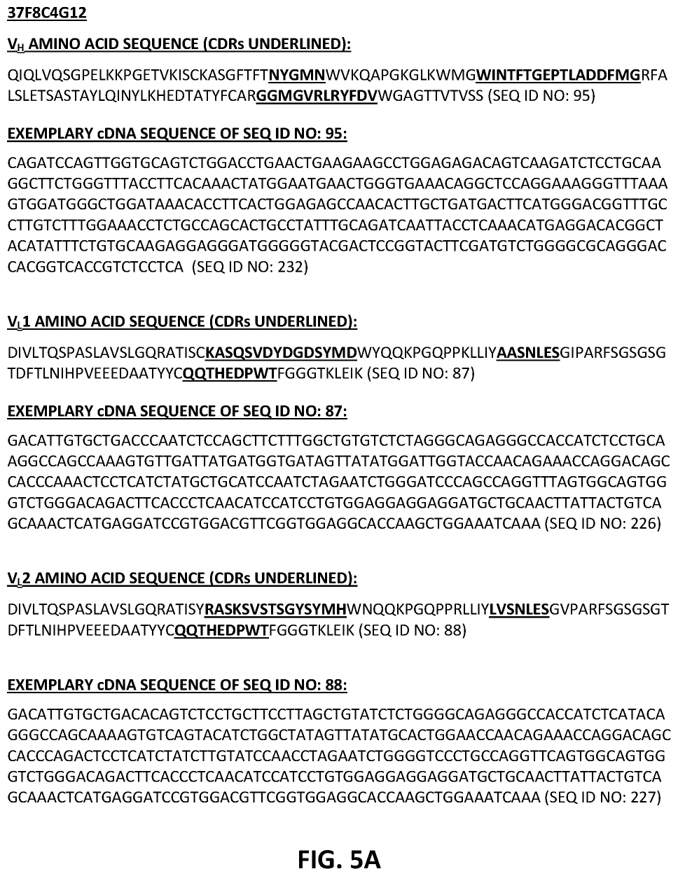

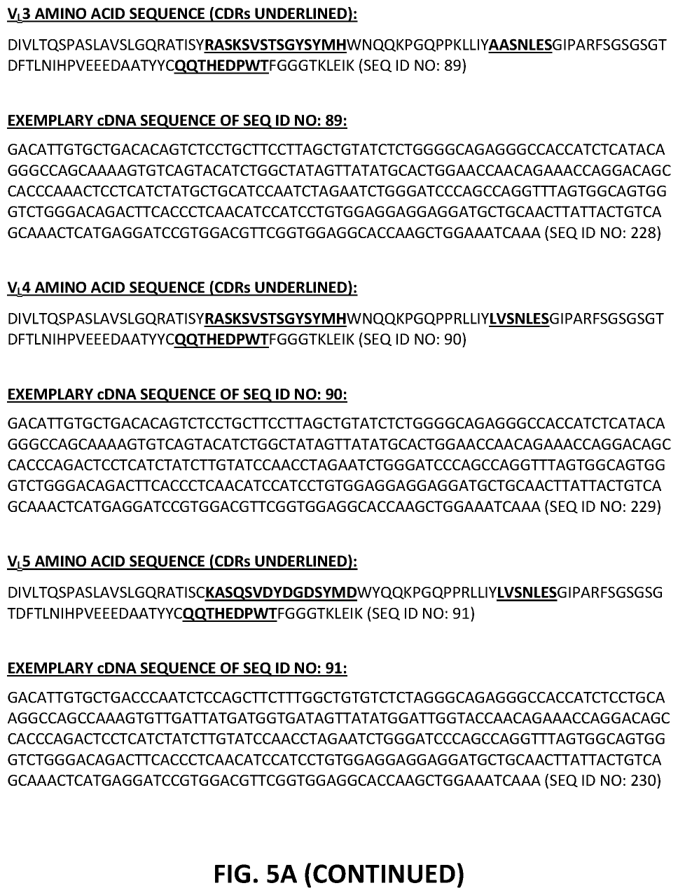

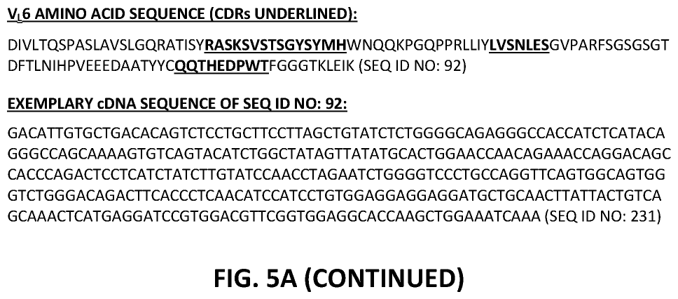

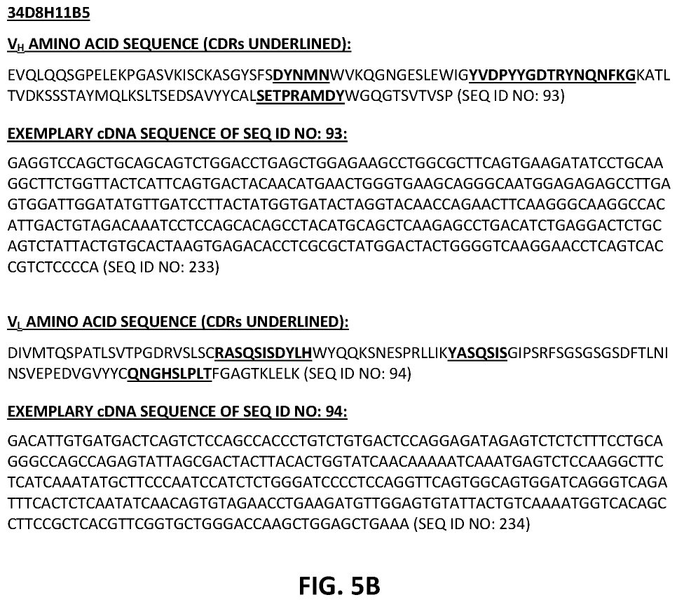

In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising AASNLES (SEQ ID NO: 105); and an LCDR3 comprising QQTHEDPWT (SEQ ID NO: 220). See, e.g., FIG. 5A. In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising QQTHEDPWT (SEQ ID NO: 220). See, e.g., FIG. 5A. In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising AASNLES (SEQ ID NO: 105); and an LCDR3 comprising QQTHEDPWT (SEQ ID NO: 220). See, e.g., FIG. 5A. In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising LVSNLES (SEQ ID NO: 108); and an LCDR3 comprising QQTHEDPWT (SEQ ID NO: 220). See, e.g., FIG. 5A. In some embodiments, the CDRs of the therapeutic anti-CD47 antibody are defined according to the Kabat numbering system. In some embodiments, the anti-idiotypic antibody (or immunologically active fragment thereof) comprises a heavy chain variable domain (VH) comprising an amino acid sequence that has at least about 95%, 96%, 97%, 98%, 99%, or 100% sequence identity to an amino acid sequence set forth in SEQ ID NO: 95. In some embodiments, the anti-idiotypic antibody (or immunologically active fragment thereof) comprises (such as further comprises) a light chain variable domain (VL) comprising an amino acid sequence that has at least about 95%, 96%, 97%, 98%, 99%, or 100% sequence identity to an amino acid sequence set forth in any one of SEQ ID NOs: 87-92. The amino acid sequences of SEQ ID NOs: 87-92 and 95 are provided in FIG. 5A. SEQ ID NOs: 226-232, which are exemplary cDNA sequences of 87-92 and 95, respectively, are also provided in FIG. 5A. In some embodiments, the anti-idiotypic antibody (or immunologically active fragment thereof) comprises 3 CDRs of a VH domain comprising SEQ ID NO: 95. Additionally or alternatively, in some embodiments, the anti-idiotypic antibody (or immunologically active fragment thereof) comprises 3 CDRs of a VL domain comprising any one of SEQ ID NOs: 87-92. In some embodiments, the 3 CDRs of the VH domain are CDRs according to Kabat, Chothia, AbM or Contact numbering scheme. Additionally or alternatively, in some embodiments, the 3 CDRs of the VL domain are CDRs according to Kabat, Chothia, AbM or Contact numbering scheme. In some embodiments, the VH domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 95, and the VL domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 87. In some embodiments, the VH domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 95, and the VL domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 88. In some embodiments, the VH domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 95, and the VL domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 89. In some embodiments, the VH domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 95, and the VL domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 90. In some embodiments, the VH domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 95, and the VL domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 91. In some embodiments, the VH domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 95, and the VL domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 92.

In some embodiments, the anti-idiotypic antibody (or the immunologically active fragment thereof) comprises a V_{H} domain that comprises an HCDR1 comprising DYNMN (SEQ ID NO: 100); an HCDR2 comprising YVDPYYGDTRYNQNFKG (SEQ ID NO: 235); and an HCDR3 comprising SETPRAMDY (SEQ ID NO: 236); and a light chain variable (V_{L}) domain that comprises an LCDR1 comprising RASQSISDYLH (SEQ ID NO: 237); an LCDR2 comprising YASQSIS (SEQ ID NO: 238); and an LCDR3 comprising QNGHSLPLT (SEQ ID NO: 239). See, e.g., FIG. 5B. In some embodiments, the CDRs of the therapeutic anti-CD47 antibody are defined according to the Kabat numbering system. In some embodiments, the anti-idiotypic antibody (or immunologically active fragment thereof) comprises a heavy chain variable domain (VH) comprising an amino acid sequence that has at least about 95%, 96%, 97%, 98%, 99%, or 100% sequence identity to an amino acid sequence set forth in SEQ ID NO: 93. In some embodiments, the anti-idiotypic antibody (or immunologically active fragment thereof) comprises (such as further comprises) a light chain variable domain (VL) comprising an amino acid sequence that has at least about 95%, 96%, 97%, 98%, 99%, or 100% sequence identity to an amino acid sequence set forth in SEQ ID NO: 94. The amino acid sequences of SEQ ID NOs: 93-94 are provided in FIG. 5B. SEQ ID NOs: 233-234, which are exemplary cDNA sequences of 93-94, respectively, are also provided in FIG. 5B. In some embodiments, the anti-idiotypic antibody (or immunologically active fragment thereof) comprises 3 CDRs of a VH domain comprising SEQ ID NO: 93. Additionally or alternatively, in some embodiments, the anti-idiotypic antibody (or immunologically active fragment thereof) comprises 3 CDRs of a VL domain comprising SEQ ID NO: 94. In some embodiments, the 3 CDRs of the VH domain are CDRs according to Kabat, Chothia, AbM or Contact numbering scheme. Additionally or alternatively, in some embodiments, the 3 CDRs of the VL domain are CDRs according to Kabat, Chothia, AbM or Contact numbering scheme. In some embodiments, the VH domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 93, and the VL domain of the anti-idiotypic antibody (or immunologically active fragment thereof) comprises an amino acid sequence that is at least about 95%, 96%, 97%, 98%, 99%, or 100% identity to the amino acid sequence set forth in SEQ ID NO: 94.

In some embodiments, the anti-idiotypic antibody is a full length antibody. In some embodiments the full-length anti-idiotypic antibody comprises a human Fc region (e.g., a human IgG Fc region, such as a human IgG1, IgG2, IgG3, or IgG4 Fc region). In some embodiments, the full-length anti-idiotypic antibody does not comprises a human Fc region (e.g., a human IgG Fc region, such as a human IgG1, IgG2, IgG3, or IgG4 Fc region). In some embodiments, the anti-idiotypic antibody comprises a non-human Fc region, e.g., without limitation, a goat, pig, rat, mouse, or chicken Fc region. In some embodiments, an anti-idiotypic antibody of the present disclosure is an antibody fragment, including without limitation a Fab, F(ab′)2, Fab′-SH, Fv, or scFv fragment, or a single domain, single heavy chain, or single light chain antibody.

Antibody fragments can be generated, e.g., by enzymatic digestion or by recombinant techniques. In some embodiments, Proteolytic digestion of an intact antibody is used to generate an antibody fragment, e.g., as described in Morimoto et al., Journal of Biochemical and Biophysical Methods 24:107-117 (1992) and Brennan et al., Science, 229:81 (1985). In some embodiments, an antibody fragment is produced by a recombinant host cell. For example, Fab, Fv and ScFv antibody fragments are expressed by and secreted from E. coli. Antibody fragments can alternatively be isolated from an antibody phage library. Fab′-SH fragments can be directly recovered from E. coli and chemically coupled to form F(ab′)₂ fragments. See Carter et al., Bio/Technology 10:163-167 (1992). F(ab′)₂ fragments can also be isolated directly from a recombinant host cell culture. Fab and F(ab′)₂ fragment with increased in vivo half-life comprising salvage receptor binding epitope residues are described in U.S. Pat. No. 5,869,046. In some embodiments, an antibody is a single chain Fv fragment (scFv). See WO 93/16185 and U.S. Pat. Nos. 5,571,894 and 5,587,458. The antibody fragment may also be a “linear antibody”, e.g., as described in U.S. Pat. No. 5,641,870, for example.

Subjects Under Treatment with an Therapeutic Anti-CD47 Antibody

A subject who is “under treatment with a therapeutic anti-CD47 antibody” refers to a subject who has been administered with at least one dose of an anti-CD47 antibody, e.g., by intravenous administration, for the treatment of a disease or disorder associated with aberrant CD47 expression (e.g., CD47 overexpression). In some embodiments, the subject has been administered with a therapeutic anti-CD47 antibody within about any one of, e.g., 5, 10, 15, 30, 45, or 60 minutes, 1, 2, 3, 4, 5, 6, 7, 8, 9, 10, 11, 12, 13, 14, 15, 16, 17, 18, 19, 20, 21, 22, 23, or 24 hours, 1, 2, 3, 4, 5, 6, or 7 days, 1, 2, 3, 4, 5, 6, 7, or 8 weeks, or 1, 2, 3, 4, 5, or 6 or 12 months. In some embodiments, the subject has cancer. In some embodiments, the cancer is a hematological cancer. In some embodiments, the hematological cancer is non-Hodgkin lymphoma (NHL), follicular lymphoma (FL), diffuse large B-cell lymphoma (DLBCL), mantle cell lymphoma (MCL), acute myeloid leukemia (AML), chronic myeloid leukemia (CML), acute lymphoblastic leukemia (ALL) or chronic lymphoblastic leukemia (CLL). In some embodiments, the cancer is solid tumor (such as lung cancer, ovarian cancer, colorectal cancer, pancreatic cancer, sarcoma cancer, head and neck cancer, gastric cancer, renal cancer, or skin cancer, etc.). In some embodiments, the cancer is a relapsed cancer (e.g., a cancer that has relapsed or recurred during or following a prior treatment for the cancer) and/or refractory cancer (e.g., a cancer that is refractory or not responsive to a prior treatment for cancer). In some embodiments, the subject is under treatment with the therapeutic anti-CD47 antibody as a single agent. In some embodiments, the subject is under treatment with the therapeutic anti-CD47 antibody in combination with at least one additional anti-cancer agent (e.g., chemotherapeutic agent, therapeutic antibody, etc.).

Exemplary Methods

In some embodiments, provided is a method of reducing interference of a therapeutic anti-CD47 antibody in a serological assay using a blood sample (e.g., a non-hemolyzed blood sample, a plasma sample, a clotted blood sample, or a serum sample) from a subject under treatment of the therapeutic anti-CD47 antibody that comprises adding an anti-idiotypic antibody that specifically recognizes an antigen binding portion of the therapeutic anti-CD47 antibody to the blood sample. In some embodiments, the method further comprises the step of conducting the serological assay. In some embodiments, provided is a method of conducting a serological assay using a blood sample (e.g., a non-hemolyzed blood sample, a plasma sample, a clotted blood sample, or a serum sample) from a subject under treatment with a therapeutic anti-CD47 antibody, comprising adding an anti-idiotypic antibody that specifically recognizes an antigen binding portion of the therapeutic anti-CD47 antibody to the subject's blood sample and conducting the serological assay on the blood sample. In some embodiments, the anti-idiotypic antibody is added to the blood sample in an amount sufficient to achieve a molar ratio of between about 1:1 and about 5:1 (such as a ratio of about 2:1 or 2.5:1) of anti-idiotypic antibody relative to therapeutic anti-CD47 antibody in the blood sample. In some embodiments, the concentration of the therapeutic anti-CD47 antibody in the blood sample is between about 20 μg/ml and about 1500 μg/ml. In some embodiments, the method comprises (such as further comprises) incubating the blood sample and the anti-idiotypic antibody for at least about 15 minutes, e.g., at 37° C., prior to conducting the serological assay. In some embodiments, the method comprises (such as further comprises) adding an enhancer (e.g., low ionic strength saline (LISS), polyethylene glycol (PEG), saline, and albumin) to the blood sample prior to conducting the serological assay. In some embodiments, the enhancer is LISS or PEG. In some embodiments, the enhancer is added to the blood sample prior to the addition of the anti-idiotypic antibody and/or prior to conducting the serological assay, and/or prior to the agglutination step of the serological assay (e.g., prior to the addition of an agglutination agent, such as anti-human globulin (AHG), to the blood sample). In some embodiments, the blood sample is treated with EDTA. In some embodiments, the serological assay is a direct antiglobulin test (DAT), an indirect antiglobulin test (IAT), an ABO test, an Rh(D) blood typing test, blood cross matching, and/or a coombs test. In some embodiments, the method comprises (such as further comprises) comprises transfusing donor blood to the subject, wherein the donor blood is determined to be compatible with the subject, according to the results of the serological assay.

In some embodiments, the subject has been administered with at least one dose a therapeutic anti-CD47 antibody (e.g., for the treatment of an CD47-associated disease or disorder) within about any one of, e.g., 5, 10, 15, 30, 45, or 60 minutes, 1, 2, 3, 4, 5, 6, 7, 8, 9, 10, 11, 12, 13, 14, 15, 16, 17, 18, 19, 20, 21, 22, 23, or 24 hours, 1, 2, 3, 4, 5, 6, or 7 days, 1, 2, 3, 4, 5, 6, 7, or 8 weeks, or 1, 2, 3, 4, 5, or 6 or 12 months. In some embodiments, the subject has cancer. In some embodiments, the cancer is a hematological cancer. In some embodiments, the hematological cancer is non-Hodgkin lymphoma (NHL), follicular lymphoma (FL), diffuse large B-cell lymphoma (DLBCL), mantle cell lymphoma (MCL), acute myeloid leukemia (AML), chronic myeloid leukemia (CML), acute lymphoblastic leukemia (ALL) or chronic lymphoblastic leukemia (CLL). In some embodiments, the cancer is solid tumor (such as lung cancer, ovarian cancer, colorectal cancer, pancreatic cancer, sarcoma cancer, head and neck cancer, gastric cancer, renal cancer, or skin cancer, etc.). In some embodiments, the cancer is a relapsed cancer (e.g., a cancer that has relapsed or recurred during or following a prior treatment for the cancer) and/or refractory cancer (e.g., a cancer that is refractory or not responsive to a prior treatment for cancer). In some embodiments, the subject is under treatment with the therapeutic anti-CD47 antibody as a single agent.

In some embodiments, the anti-idiotypic antibody binds to a therapeutic anti-CD47 antibody that comprises an HCDR1, an HCDR2 and an HCDR3 as set forth in a VH that comprises SEQ ID NO: 79 and an LCDR1, an LCDR2 and an LCDR3 as set forth in a V_{L} that comprises SEQ ID NO: 80. In some embodiments, the therapeutic anti-CD47 antibody comprises a VH domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the CDRs of the therapeutic anti-CD47 antibody are defined according to the Kabat numbering system. In some embodiments, the therapeutic anti-CD47 antibody comprises a V_{H} that comprises SEQ ID NO: 79 a V_{L} that comprises SEQ ID NO: 80. In some embodiments, the therapeutic anti-CD47 antibody comprises a VH that comprises SEQ ID NO: 79 a V_{L} that comprises SEQ ID NO: 80. In some embodiments, the therapeutic anti-CD47 antibody is a full-length antibody. In some embodiments, the therapeutic anti-CD47 comprises (such as further comprises) a human IgG4 Fc region or a variant thereof comprising an S228P substitution (wherein amino numbering is according to the EU index. In some embodiments, the therapeutic anti-CD47 comprises a heavy chain comprising the amino acid sequence of SEQ ID NO: 216 and a light chain comprising the amino acid sequence of SEQ ID NO: 218. In some embodiments, the therapeutic anti-CD47 comprises a heavy chain comprising the amino acid sequence of SEQ ID NO: 217 and a light chain comprising the amino acid sequence of SEQ ID NO: 218.

In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106). In some embodiments, the anti-idiotypic antibody (or the immunologically active fragment thereof) comprises a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109). In some embodiments, the CDRs of the anti-idiotypic antibody (or immunologically active fragment thereof) are defined according to the Kabat numbering system. In some embodiments, the anti-idiotypic antibody (or immunologically active fragment thereof) comprises a heavy chain variable domain (VH) comprising an amino acid sequence that has at least about 95%, 96%, 97%, 98%, 99%, or 100% sequence identity to an amino acid sequence set forth in SEQ ID NO: 110, and, optionally, a light chain variable domain (VL) comprising an amino acid sequence that has at least about 95%, 96%, 97%, 98%, 99%, or 100% sequence identity to an amino acid sequence set forth in any one of SEQ ID NOs: 111-114. In some embodiments, the anti-idiotypic antibody (or immunologically active fragment thereof) comprises 3 CDRs of a VH domain comprising SEQ ID NO: 110. Additionally or alternatively, in some embodiments, the anti-idiotypic antibody (or immunologically active fragment thereof) comprises 3 CDRs of a VL domain comprising any one of SEQ ID NOs: 111-114. The amino acid sequences of SEQ ID NOs: 110-114 are provided in FIG. 2. In some embodiments, the 3 CDRs of the VH domain are CDRs according to Kabat, Chothia, AbM or Contact numbering scheme. Additionally or alternatively, in some embodiments, the 3 CDRs of the VL domain are CDRs according to Kabat, Chothia, AbM or Contact numbering scheme. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO:111. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO:112. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO:113. In some embodiments, the VH domain of the anti-idiotypic antibody comprises an amino acid sequence that is at least about 95%, 96%, 97%, 98%, 99%, or 100% identity to the amino acid sequence set forth in SEQ ID NO:110, and the VL domain of the anti-idiotypic antibody comprises an amino acid sequence that is at least about 95%, 96%, 97%, 98%, 99%, or 100% identity to the amino acid sequence set forth in SEQ ID NO:114.

Methods of Detecting a Therapeutic Anti-CD47 Antibody in a Sample from a Subject Under Treatment with the Therapeutic Anti-CD47

In some embodiments, provided is a method of detecting an anti-CD47 antibody (e.g., a therapeutic anti-CD47 antibody) or an immunologically active fragment thereof in a sample (e.g., a blood or tissue sample) from a subject, comprising contacting the sample with an anti-idiotypic antibody described herein (or an immunologically active fragment thereof), and detecting a complex comprising the anti-idiotypic antibody and the anti-CD47 antibody or fragment thereof, thereby detecting the presence of anti-CD47 antibody or fragment thereof. In some embodiments, the assay is, e.g., a western blot analysis, an immunoprecipitation, a molecular binding assay, an ELISA, an ELIFA, or a fluorescence activated cell sorting assay. In some embodiments, the sample is a fresh sample. In some embodiments, the sample is a fixed (e.g., formalin-fixed or paraffin-embedded sample).

Nucleic Acids and Vectors Encoding Anti-Idiotypic Antibodies

Nucleic acid molecules encoding the anti-idiotypic antibodies (or immunologically active fragments thereof) described herein are also contemplated. Exemplary nucleic acid sequences of the V_{H} and V_{L} domains of the anti-idiotypic antibodies (or immunologically active fragments thereof) described herein are provided in FIGS. 2, 5A, and 5B. In some embodiments, provided is a nucleic acid (or a set of nucleic acids) encoding an anti-idiotypic antibody, including any of the anti-idiotypic antibodies described herein. In some embodiments, the nucleic acid (or set of nucleic acids) encoding an anti-idiotypic antibody described herein may further comprises a nucleic acid sequence encoding a peptide tag (such as protein purification tag, e.g., His-tag, HA tag). In some embodiments, the nucleic acid (or set of nucleic acids) encoding an anti-idiotypic antibody (or an immunologically active fragment thereof) comprises a leader sequence. In some embodiments, provided are nucleic acids comprising nucleotide sequences that hybridize to the nucleic acid sequences encoding an anti-idiotypic antibody described herein under at least moderately stringent hybridization conditions.

Also provided are vectors in which a nucleic acid described herein is inserted.

In brief summary, the expression of an anti-idiotypic antibody (or antigen binding fragment thereof) by a natural or synthetic nucleic acid encoding the anti-idiotypic antibody (or antigen binding fragment thereof) can be achieved by inserting the nucleic acid into an appropriate expression vector, such that the nucleic acid is operably linked to 5′ and 3′ regulatory elements, including for example a promoter (e.g., a constitutive, regulatable, tissue-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the nucleic acid(s) encoding the anti-idiotypic antibody (or immunologically active fragment thereof) is inducible. In some embodiments, the nucleic acid(s) encoding the anti-idiotypic antibody (or immunologically active fragment thereof) is operably linked to an inducible promoter, including any inducible promoter known in the art. In some embodiments, the nucleic acid(s) encoding the anti-idiotypic antibody described herein has been engineered to encode an epitope tag, e.g., to facilitate purification or detection of the antibody. Exemplary epitope tags include, but are not limited to, e.g., 6×His (also known as His-tag or hexahistidine tag), FLAG, HA, Myc, V5, GFP (green fluorescent protein, e.g., enhanced green fluorescent protein or EGFP), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein).

Methods of Antibody Production

An anti-idiotypic antibody (or immunologically active fragment thereof) of the present disclosure may be produced by any means known in the art. Exemplary techniques for antibody production are described below; however these exemplary techniques are provided for illustrative purposes only and are not intended to be limiting. In addition, exemplary antibody properties contemplated for use with the antibodies described herein are further described.

To prepare an antigen, the antigen may be purified or otherwise obtained from a natural source, or it may be expressed using recombinant techniques. In some embodiments, the antigen may be used as a soluble protein. In some embodiments, the antigen may be conjugate to another polypeptide or other moiety, e.g., to increase its immunogenicity. For example, an antigen described herein may be coupled with an Fc region. In some embodiments, a cell expressing the antigen on its cell surface may be used as the antigen.

Polyclonal antibodies can be raised in an animal by multiple subcutaneous (sc) or intraperitoneal (ip) injections of the antigen and an adjuvant. For example, descriptions of chicken immunization are described herein. In some embodiments, the antigen is conjugated with an immunogenic protein, e.g., keyhole limpet hemocyanin, serum albumin, bovine thyroglobulin, or soybean trypsin inhibitor using a bifunctional or derivatizing agent. Exemplary methods for immunization of chickens are provided herein. Relevant methods suitable for a variety of other organisms, such as mammals, are well known in the art.

As described supra, monoclonal antibodies may be produced by a variety of methods. In some embodiments, a monoclonal antibody of the present disclosure is made using the hybridoma method first described by Kohler et al., Nature, 256:495 (1975), and further described in Hongo et al., Hybridoma, 14 (3): 253-260 (1995); Harlow et al., Antibodies: A Laboratory Manual, (Cold Spring Harbor Laboratory Press, 2nd ed. 1988); and Hammerling et al., in: Monoclonal Antibodies and T-Cell Hybridomas 563-681 (Elsevier, N.Y., 1981). Human hybridoma technology (Trioma technology) is described in Vollmers and Brandlein, Histology and Histopathology, 20(3):927-937 (2005) and Vollmers and Brandlein, Methods and Findings in Experimental and Clinical Pharmacology, 27(3):185-91 (2005). A culture medium in which hybridoma cells are grown may be screened for the presence of an antibody of interest, e.g., by in vitro binding assay, immunoprecipitation, ELISA, RIA, etc.; and the binding affinity may be determined, e.g., by Scatchard analysis. A hybridoma that produces an antibody with desired binding properties can be subcloned and grown using known culture techniques, grown in vivo as ascites tumors in an animal, and the like.

In some embodiments, an anti-idiotypic antibody is made using a library method, such as a phage display library. See, e.g., Hoogenboom et al. in Methods in Molecular Biology 178:1-37 (O'Brien et al., ed., Human Press, Totowa, N J, 2001). In some embodiments, repertoires of VH and VL genes are cloned by polymerase chain reaction (PCR) and recombined randomly in phage libraries, which are then screened for antigen-binding phage, e.g., as described in Winter et al., Ann. Rev. Immunol., 12: 433-455 (1994). Phage typically display antibody fragments, either as single-chain Fv (scFv) fragments or as Fab fragments. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992).

Antibodies can be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

An antibody of the present disclosure can be produced recombinantly as a fusion polypeptide with a heterologous polypeptide, e.g., a signal sequence or other polypeptide having a specific cleavage site at the N-terminus of the mature protein or polypeptide. The heterologous signal sequence selected can be one that is recognized and processed (e.g., cleaved by a signal peptidase) by the host cell. For prokaryotic host cells that do not recognize and process a native antibody signal sequence, the signal sequence is substituted by a prokaryotic signal sequence selected, for example, from alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, etc. In mammalian cell expression, mammalian signal sequences as well as viral secretory leaders, for example, the herpes simplex gD signal, are available.

Both expression and cloning vectors contain a nucleic acid sequence that enables the vector to replicate in one or more selected host cells, e.g., to allow the vector to replicate independently of the host chromosomal DNA. This sequence can include origins of replication or autonomously replicating sequences. Such sequences are well known for a variety of bacteria, yeast, and viruses. Generally, the origin of replication component is not needed for mammalian expression vectors (the SV40 origin may be used because it contains the early promoter).

Expression and cloning vectors can contain a selection gene or selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media. Examples of dominant selection use the drugs neomycin, mycophenolic acid and hygromycin. Another example of suitable selectable markers for mammalian cells are those that enable the identification of cells competent to take up antibody-encoding nucleic acid, such as DHFR, glutamine synthetase (GS), thymidine kinase, metallothionein-I and -II, preferably primate metallothionein genes, adenosine deaminase, ornithine decarboxylase, and the like. For example, a Chinese hamster ovary (CHO) cell line deficient in endogenous DHFR activity transformed with the DHFR gene is identified by culturing the transformants in a culture medium containing methotrexate (Mtx), a competitive antagonist of DHFR.

Alternatively, host cells (particularly wild-type hosts that contain endogenous DHFR) transformed or co-transformed with DNA sequences encoding an antibody of interest, wild-type DHFR gene, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors generally contain a promoter that is recognized by the host organism and is operably linked to nucleic acid encoding an antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoter sequences are known for eukaryotes. Yeast promoters are well known in the art and can include inducible promoters/enhancers regulated by growth conditions. Virtually all eukaryotic genes have an AT-rich region located approximately 25 to 30 bases upstream from the site where transcription is initiated. Examples include without limitation the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Antibody transcription from vectors in mammalian host cells can be controlled, for example, by promoters obtained from the genomes of viruses. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. Alternatively, the Rous Sarcoma Virus long terminal repeat can be used as the promoter.

Transcription of a DNA encoding an antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus.

Expression vectors used in eukaryotic host cells (yeast, fungi, insect, plant, animal, human, or nucleated cells from other multicellular organisms) will also contain sequences necessary for the termination of transcription and for stabilizing the mRNA.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, etc. In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. Certain fungi and yeast strains may be selected in which glycosylation pathways have been “humanized,” resulting in the production of an antibody with a partially or fully human glycosylation pattern. See, e.g., Li et al., Nat. Biotech. 24:210-215 (2006).

Plant cell cultures of cotton, corn, potato, soybean, petunia, tomato, duckweed (Leninaceae), alfalfa (M. truncatula), and tobacco can also be utilized as hosts.

Suitable host cells for the expression of glycosylated antibody are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified.

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268.

The host cells of the present disclosure may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to one of skill in the art.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli.

The antibody composition prepared from the cells can be purified using, for example, hydroxylapatite chromatography, hydrophobic interaction chromatography, gel electrophoresis, dialysis, and affinity chromatography, with affinity chromatography being among one of the typically preferred purification steps. In some embodiments, an anti-idiotypic antibody described herein comprises an epitope tag (e.g., a tag attached to the antibody via a cleavable linker) to facilitate purification. Exemplary epitope tags include, but are not limited to, e.g., e.g., 6×His (also known as His-tag or hexahistidine tag), FLAG, HA, Myc, V5, GFP (green fluorescent protein, e.g., enhanced green fluorescent protein or EGFP), GST (glutathione-S-transferase), j-GAL (0-galactosidase), Luciferase, MBP (Maltose Binding Protein), RFP (Red Fluorescence Protein), and VSV-G (Vesicular Stomatitis Virus Glycoprotein.

Thus, in some embodiments, provided is a method of making an anti-idiotypic antibody (or an immunologically active fragment thereof) described herein comprising culturing a host cell that comprises a nucleic acid that encodes the anti-idiotypic antibody (or immunologically active fragment thereof) under conditions effective to cause expression of the antibody (or fragment); and b) recovering the anti-idiotypic antibody (or fragment thereof) expressed by the host cell. In some embodiments, the method further comprises the step of c) purifying the antibody. In some embodiments, purifying the anti-idiotypic antibody comprises at least one chromatography step, such as a Protein A or Protein L chromatography step.

Articles of Manufacture and Kits

Also provided is an article of manufacture or kit for comprising one or more anti-idiotypic antibodies described herein. In some embodiments, the article or manufacture or kit is for use in mitigating/eliminating interference in a serological caused by a therapeutic anti-CD47 antibody, e.g., according to a method described herein. In certain embodiments, the article of manufacture or kit comprises a container containing one or more of the anti-idiotypic antibodies (or immunologically active fragments thereof) described herein or compositions comprising such anti-idiotypic antibodies. In some embodiments, the kit includes one or more positive controls, for example CD47 (or fragments thereof) or CD47⁺ cells. In some embodiments, the kit includes negative controls, for example a surface or solution that is substantially free of CD47, or a cell that does not express CD47.

In certain embodiments, the article of manufacture or kit comprises a container and a label or package insert on or associated with the container. Suitable containers include, for example, bottles, vials, test tubes, etc. The containers may be formed from a variety of materials such as glass or plastic. The container holds a composition comprising one or more anti-idiotypic antibodies described herein. In some embodiments, the label or package insert indicates that the composition is used for mitigating/eliminating the interference in a serological test caused by the presence of a therapeutic anti-CD47 antibody in a subject's blood sample (e.g., a non-hemolyzed blood sample, a plasma sample, a clotted blood sample, or a serum sample).

Kits are also provided that are useful for various purposes, e.g., for isolation or detection of a therapeutic anti-CD47 antibody, e.g., in a blood sample or tissue sample obtained from a subject, optionally in combination with the articles of manufacture. For detection of a therapeutic anti-CD47 antibody, the kit can contain an anti-idiotypic antibody (or immunologically active fragment thereof) provided herein coupled to beads (e.g., sepharose beads). Kits can be provided which contain the antibodies (or fragments thereof) for detection and quantitation of a therapeutic anti-CD47 antibody in vitro, e.g., in an ELISA or a Western blot. As with the article of manufacture, the kit comprises a container and a label or package insert on or associated with the container. For example, the container holds a composition comprising at least one anti-idiotypic antibody provided herein. Additional containers may be included that contain, e.g., diluents and buffers, control antibodies. Where the antibody is labeled with an enzyme, the kit will include substrates and cofactors required by the enzyme (e.g., a substrate precursor which provides the detectable chromophore or fluorophore). The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration. The label or package insert may provide a description of the composition as well as instructions for the intended in vitro use (e.g., detecting a therapeutic anti-CD47 antibody).

Methods of Using an Anti-Human Globulin Reagent that does not Bind a Human IgG4 Fc Region to Mitigate Interference in a Pre-Transfusion Serological Assay Caused by a Therapeutic Anti-CD47 Antibody

Also provided herein are methods of reducing interference of a therapeutic anti-CD47 antibody in a serological assay using a blood sample from a subject under treatment of the therapeutic anti-CD47 antibody that comprises a human IgG4 Fc domain (or a variant thereof comprising an S228P substitution, wherein amino acid numbering is according to the EU index). In some embodiments, the method comprises conducting a serological assay on the blood sample using an anti-human globulin (AHG) reagent that does not recognize or bind an human IgG4 antibody Fc region. In some embodiments, the AHG reagent that does not recognize or bind an human IgG4 antibody Fc region comprises (or is) ANTI-IgG (MURINE MONOCLONAL)(GREEN OR UNCOLORED) GAMMA-CLONE® commercially available from Immucor (Catalog #0409203 and 0409210. In some embodiments, the serological assay is performed at room temperature (e.g., between 17° C.-25° C.). In some embodiments, the blood sample is a non-hemolyzed blood sample, a plasma sample, a clotted blood sample, or a serum sample. In some embodiments, the serological assay is, e.g., a direct antiglobulin test (DAT), an indirect antiglobulin test (IAT), an ABO test, an Rh(D) blood typing test, blood cross matching, and/or a coombs test. Further details about these, and other, serological assays are provided below. In some embodiments, the method comprises (such as further comprises) comprises transfusing donor blood to the subject, wherein the donor blood is determined to be compatible with the subject, according to the results of the serological assay. Further details regarding transfusing donor blood to a subject under treatment with a therapeutic anti-CD47 antibody are provided below.

In some embodiments, the blood sample used in the method is obtained from any one of the exemplary subjects (i.e., subjects under treatment with a therapeutic anti-CD47 antibody) described elsewhere herein. In some embodiments, the subject has been administered with a therapeutic anti-CD47 antibody (e.g., via intravenous infusion) within about any one of, e.g., 5, 10, 15, 30, 45, or 60 minutes, 1, 2, 3, 4, 5, 6, 7, 8, 9, 10, 11, 12, 13, 14, 15, 16, 17, 18, 19, 20, 21, 22, 23, or 24 hours, 1, 2, 3, 4, 5, 6, or 7 days, 1, 2, 3, 4, 5, 6, 7, or 8 weeks, or 1, 2, 3, 4, 5, or 6 or 12 months.

In some embodiments, the method comprises (such as further comprise) a step of adding an enhancer to the subject's blood sample prior to conducting the method of mitigating interference and/or the serological assay. In some embodiments, the enhancer is added to the blood sample (e.g., a non-hemolyzed blood sample, a plasma sample, a clotted blood sample, or a serum sample) prior to the addition of the anti-idiotypic antibody. Additionally or alternatively, in some embodiments, the enhancer is added to the blood sample (e.g., a non-hemolyzed blood sample, a plasma sample, a clotted blood sample, or a serum sample) after the anti-idiotypic antibody has been added but prior to conducting the serological assay. Additionally or alternatively, in some embodiments, the enhancer is added to the blood sample (e.g., a non-hemolyzed blood sample, a plasma sample, a clotted blood sample, or a serum sample) prior to the agglutination step of the serological assay (e.g., prior to the addition of an agglutination agent, such as anti-human globulin (AHG), to the blood sample). An “enhancer” refers to an agent that enhances agglutination in the serological assay and reduces incubation time, e.g., by promoting blood group antibody-antigen reactions. In some embodiments, the enhancer is low ionic strength saline (LISS) or polyethylene glycol (PEG). In some embodiments, the method comprises treating the blood sample with EDTA.

In some embodiments, the therapeutic anti-CD47 antibody comprises a V_{H} domain that comprises an HCDR1 comprising SYAMS (SEQ ID NO: 115); an HCDR2 comprising AISGSGGSTYYADSVKG (SEQ ID NO: 116); and an HCDR3 comprising YSIGRHTFDH (SEQ ID NO: 117) and a (V_{L}) domain that comprises an LCDR1 comprising TRSSGGIASNFVQ (SEQ ID NO: 118); an LCDR2 comprising RDNQRPS (SEQ ID NO: 119); and an LCDR3 comprising QSYDDHNHWV (SEQ ID NO: 120). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising GYAMT (SEQ ID NO: 124); an HCDR2 comprising AITSTGGRTYYADSVKG (SEQ ID NO: 125); and an HCDR3 comprising ESNFRAFDI (SEQ ID NO: 126) and a (V_{L}) domain that comprises an LCDR1 comprising RSSQSLLHSNGYNYLD (SEQ ID NO: 127); an LCDR2 comprising LNSNRAS (SEQ ID NO: 128); and an LCDR3 comprising MQALQIPPT (SEQ ID NO: 129). In some embodiments, the therapeutic anti-CD47 antibody comprises a V_{H} domain that comprises an HCDR1 comprising DAWMT (SEQ ID NO: 130); an HCDR2 comprising VIYSGGSTYYADSVKG (SEQ ID NO: 131); and an HCDR3 comprising GARGHPGQDY (SEQ ID NO: 132) and a (V_{L}) domain that comprises an LCDR1 comprising TRSSGTIASNFVQ (SEQ ID NO: 133); an LCDR2 comprising ENDRRPS (SEQ ID NO: 134); and an LCDR3 comprising QSYDSSTHGWV (SEQ ID NO: 135). In some embodiments, the therapeutic anti-CD47 antibody comprises a V_{H} domain that comprises an HCDR1 comprising DYYMS (SEQ ID NO: 136); an HCDR2 comprising YTSRFGSDTNYADSVKG (SEQ ID NO: 137); and an HCDR3 comprising DVHNRDAY (SEQ ID NO: 138) and a (V_{L}) domain that comprises an LCDR1 comprising SGSSSNIGGNSVS (SEQ ID NO: 139); an LCDR2 comprising RNHQRPS (SEQ ID NO: 140); and an LCDR3 comprising ATWDFSLSGFV (SEQ ID NO: 141). In some embodiments, the therapeutic anti-CD47 antibody comprises a V_{H} domain that comprises an HCDR1 comprising SYAMS (SEQ ID NO: 142); an HCDR2 comprising AISGSGGSTYYADSVKG (SEQ ID NO: 143); and an HCDR3 comprising ADY (SEQ ID NO: 144) and a (V_{L}) domain that comprises an LCDR1 comprising RASQDIRNDLD (SEQ ID NO: 145); an LCDR2 comprising AASNLQS (SEQ ID NO: 146); and an LCDR3 comprising QQSYITPPWT (SEQ ID NO: 147). In some embodiments, the therapeutic anti-CD47 antibody comprises a V_{H} domain that comprises an HCDR1 comprising SYGMS (SEQ ID NO: 148); an HCDR2 comprising TISGSGSSTNYADSVKG (SEQ ID NO: 149); and an HCDR3 comprising GRYYYDSLDAFDI (SEQ ID NO: 150) and a (V_{L}) domain that comprises an LCDR1 comprising RASQEIRTAYLA (SEQ ID NO: 151); an LCDR2 comprising YASSRAT (SEQ ID NO: 152); and an LCDR3 comprising QQYDTSPPT (SEQ ID NO: 153). In some embodiments, the therapeutic anti-CD47 antibody comprises a V_{H} domain that comprises an HCDR1 comprising SYAMS (SEQ ID NO: 115); an HCDR2 comprising AISGTGGSTYYADSVKG (SEQ ID NO: 154); and an HCDR3 comprising DKWSSWPTYYFDY (SEQ ID NO: 155) and a (V_{L}) domain that comprises an LCDR1 comprising TRSSGSIASNYVQ (SEQ ID NO: 156); an LCDR2 comprising EDNQRPS (SEQ ID NO: 157); and an LCDR3 comprising QSYDSSNVI (SEQ ID NO: 158). In some embodiments, the therapeutic anti-CD47 antibody comprises a V_{H} domain that comprises an HCDR1 comprising SYSMA (SEQ ID NO: 159); an HCDR2 comprising AVSNSGVETYYADSVKG (SEQ ID NO: 160); and an HCDR3 comprising RTRQLLTPREFDY (SEQ ID NO: 161) and a (V_{L}) domain that comprises an LCDR1 comprising RASQDITRWLA (SEQ ID NO: 162); an LCDR2 comprising DASSLQS (SEQ ID NO: 163); and an LCDR3 comprising QQGSSVPFT (SEQ ID NO: 164). In some embodiments, the therapeutic anti-CD47 antibody comprises a V_{H} domain that comprises an HCDR1 comprising NYAMS (SEQ ID NO: 165); an HCDR2 comprising SVSSAGGSTYYADSVKG (SEQ ID NO: 166); and an HCDR3 comprising RVNRAFDL (SEQ ID NO: 167) and a (V_{L}) domain that comprises an LCDR1 comprising RASQSVSSSYLA (SEQ ID NO: 168); an LCDR2 comprising GASSRAT (SEQ ID NO: 169); and an LCDR3 comprising QQYGSSPPMYT (SEQ ID NO: 170). In some embodiments, the therapeutic anti-CD47 antibody comprises a V_{H} domain that comprises an HCDR1 comprising NAWMS (SEQ ID NO: 171); an HCDR2 comprising RIKSKTDGGTTDYAAPVKG (SEQ ID NO: 172); and an HCDR3 comprising DKSYGYTFDY (SEQ ID NO: 173) and a (V_{L}) domain that comprises an LCDR1 comprising SGSGSNIGSNSVH (SEQ ID NO: 174); an LCDR2 comprising TNNQRPS (SEQ ID NO: 175); and an LCDR3 comprising ATWDDRLSGPV (SEQ ID NO: 176). In some embodiments, the therapeutic anti-CD47 antibody comprises a V_{H} domain that comprises an HCDR1 comprising SYWMH (SEQ ID NO: 177); an HCDR2 comprising AISGSGAGTYYPDSVKG (SEQ ID NO: 178); and an HCDR3 comprising DRSLSFGFDI (SEQ ID NO: 179) and a (V_{L}) domain that comprises an LCDR1 comprising TRSSGSIGSTYVQ (SEQ ID NO: 180); an LCDR2 comprising KDDQRPS (SEQ ID NO: 181); and an LCDR3 comprising QSSDTSNLV (SEQ ID NO: 182). In some embodiments, the therapeutic anti-CD47 antibody comprises a V_{H} domain that comprises an HCDR1 comprising RYWMS (SEQ ID NO: 183); an HCDR2 comprising NIKGDGSQTYYADSVKG (SEQ ID NO: 184); and an HCDR3 comprising GAAYHINSWLDP (SEQ ID NO: 185) and a (V_{L}) domain that comprises an LCDR1 comprising RASQSISGNYLA (SEQ ID NO: 186); an LCDR2 comprising GAFRRAT (SEQ ID NO: 187); and an LCDR3 comprising QHYNNFPHT (SEQ ID NO: 188). In some embodiments, the therapeutic anti-CD47 antibody comprises a V_{H} domain that comprises an HCDR1 comprising HAWMN (SEQ ID NO: 189); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYQVNNRNYLA (SEQ ID NO: 190); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQTVLYPLNNRNYLA (SEQ ID NO: 97); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSSDI (SEQ ID NO: 192) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSSDI (SEQ ID NO: 193) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSSDI (SEQ ID NO: 194) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDA (SEQ ID NO: 195) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDS (SEQ ID NO: 196) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SDRASDK (SEQ ID NO: 98) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDT (SEQ ID NO: 197) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSQDI (SEQ ID NO: 198) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSQDI (SEQ ID NO: 199)) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSQDI (SEQ ID NO: 200) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLRPPLN (SEQ ID NO: 202). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLTPPLN (SEQ ID NO: 99). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLS (SEQ ID NO: 203). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLA (SEQ ID NO: 204). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLNAPLH (SEQ ID NO: 205). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLEAPLV (SEQ ID NO: 206). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLH (SEQ ID NO: 207). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QRLIAPPFT (SEQ ID NO: 208). In some embodiments, the therapeutic anti-CD47 antibody comprises a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLA (SEQ ID NO: 209). In some embodiments, the therapeutic anti-CD47 antibody comprises a V_{H} domain that comprises an HCDR1 comprising SYYMH (SEQ ID NO: 210); an HCDR2 comprising EINPNNARINFNEKFKT (SEQ ID NO: 211); and an HCDR3 comprising GYYRYGAWFGY (SEQ ID NO: 212) and a (V_{L}) domain that comprises an LCDR1 comprising RASQDISDYLN (SEQ ID NO: 213); an LCDR2 comprising YISRLHS (SEQ ID NO: 214); and an LCDR3 comprising QQGHTLPWT (SEQ ID NO: 215). In some embodiments, the therapeutic anti-CD47 antibody comprises a V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the CDRs of the therapeutic anti-CD47 antibody are defined according to the Kabat numbering system (Kabat et al., Sequences of Proteins of Immunological Interest, 5th Ed. Public Health Service, National Institutes of Health, Bethesda, Md. (1991)).

In some embodiments, the therapeutic anti-CD47 antibody comprises a V_{H} that comprises SEQ ID NO: 1 and an LCDR1, an LCDR2 and a VL that comprises SEQ ID NO: 2. In some embodiments, the therapeutic anti-CD47 comprises a V_{H} that comprises SEQ ID NO: 3 and a V_{L} that comprises SEQ ID NO: 4. In some embodiments, the therapeutic anti-CD47 comprises a V_{H} that comprises SEQ ID NO: 5 and a V_{L} that comprises SEQ ID NO: 6. In some embodiments, the therapeutic anti-CD47 antibody comprises a V_{H} that comprises SEQ ID NO: 7 and a V_{L} that comprises SEQ ID NO: 8. In some embodiments, the therapeutic anti-CD47 comprises a V_{H} that comprises SEQ ID NO: 9 and a V_{L} that comprises SEQ ID NO: 10. In some embodiments, the therapeutic anti-CD47 antibody comprises a V_{H} that comprises SEQ ID NO:11 and an LCDR1, an LCDR2 and an LCDR3 as set forth in a V_{L} that comprises SEQ ID NO: 12. In some embodiments, the therapeutic anti-CD47 antibody comprises a V_{H} that comprises SEQ ID NO: 13 and a V_{L} that comprises SEQ ID NO: 14. In some embodiments, the therapeutic anti-CD47 antibody comprises a V_{H} that comprises SEQ ID NO:15 and a V_{L} that comprises SEQ ID NO: 16. In some embodiments, the therapeutic anti-CD47 antibody comprises V_{H} that comprises SEQ ID NO: 17 and a V_{L} that comprises SEQ ID NO: 18. In some embodiments, the therapeutic anti-CD47 antibody comprises a V_{H} that comprises SEQ ID NO: 19 and a V_{L} that comprises SEQ ID NO: 20. In some embodiments, the therapeutic anti-CD47 antibody comprises a V_{H} that comprises SEQ ID NO: 21 and a V_{L} that comprises SEQ ID NO: 22. In some embodiments, the therapeutic anti-CD47 antibody comprises a V_{H} that comprises SEQ ID NO:23 and a V_{L} that comprises SEQ ID NO: 24. In some embodiments, the therapeutic anti-CD47 antibody comprises in a V_{H} that comprises SEQ ID NO: 25 and a V_{L} that comprises SEQ ID NO: 26. In some embodiments, the therapeutic anti-CD47 antibody comprises a V_{H} that comprises SEQ ID NO: 27 and a V_{L} that comprises SEQ ID NO: 28. In some embodiments, the therapeutic anti-CD47 antibody comprises a V_{H} that comprises SEQ ID NO: 29 and a V_{L} that comprises SEQ ID NO: 30. In some embodiments, the therapeutic anti-CD47 antibody comprises a V_{H} that comprises SEQ ID NO: 31 and a V_{L} that comprises SEQ ID NO: 32. In some embodiments, the therapeutic anti-CD47 antibody a V_{H} that comprises SEQ ID NO: 33 and a V_{L} that comprises SEQ ID NO: 34. In some embodiments, the therapeutic anti-CD47 antibody comprises a V_{H} that comprises SEQ ID NO: 35 and a V_{L} that comprises SEQ ID NO: 36. In some embodiments, the therapeutic anti-CD47 antibody comprises a V_{H} that comprises SEQ ID NO: 37 and a V_{L} that comprises SEQ ID NO: 38. In some embodiments, the therapeutic anti-CD47 antibody comprises a V_{H} that comprises SEQ ID NO: 39 and a V_{L} that comprises SEQ ID NO: 40. In some embodiments, the therapeutic anti-CD47 antibody comprises a V_{H} that comprises SEQ ID NO: 41 and a V_{L} that comprises SEQ ID NO: 42. In some embodiments, the therapeutic anti-CD47 antibody a V_{H} that comprises SEQ ID NO: 43 and a V_{L} that comprises SEQ ID NO: 44. In some embodiments, the therapeutic anti-CD47 antibody comprises a V_{H} that comprises SEQ ID NO: 45 and a V_{L} that comprises SEQ ID NO: 46. In some embodiments, the therapeutic anti-CD47 antibody comprises a V_{H} that comprises SEQ ID NO: 47 and a V_{L} that comprises SEQ ID NO: 48. In some embodiments, the therapeutic anti-CD47 antibody comprises a V_{H} that comprises SEQ ID NO: 49 and a V_{L} that comprises SEQ ID NO: 50. In some embodiments, the therapeutic anti-CD47 antibody comprises a V_{H} that comprises SEQ ID NO: 51 and a V_{L} that comprises SEQ ID NO: 52. In some embodiments, the therapeutic anti-CD47 comprises a V_{H} that comprises SEQ ID NO: 53 and a V_{L} that comprises SEQ ID NO: 54. In some embodiments, the therapeutic anti-CD47 antibody comprises a V_{H} that comprises SEQ ID NO: 55 and a V_{L} that comprises SEQ ID NO: 56. In some embodiments, the therapeutic anti-CD47 antibody comprises a V_{H} that comprises SEQ ID NO: 57 and a V_{L} that comprises SEQ ID NO: 58. In some embodiments, the therapeutic anti-CD47 antibody comprises a V_{H} that comprises SEQ ID NO: 59 and a V_{L} that comprises SEQ ID NO: 60. In some embodiments, the therapeutic anti-CD47 antibody comprises a V_{H} that comprises SEQ ID NO: 61 and a V_{L} that comprises SEQ ID NO: 62. In some embodiments, the therapeutic anti-CD47 antibody comprises a V_{H} that comprises SEQ ID NO: 63 and a V_{L} that comprises SEQ ID NO: 64. In some embodiments, the therapeutic anti-CD47 antibody comprises a V_{H} that comprises SEQ ID NO: 65 and a V_{L} that comprises SEQ ID NO: 66. In some embodiments, the therapeutic anti-CD47 antibody comprises a V_{H} that comprises SEQ ID NO: 67 and a V_{L} that comprises SEQ ID NO: 68. In some embodiments, the therapeutic anti-CD47 antibody comprises a V_{H} that comprises SEQ ID NO: 69 and a V_{L} that an comprises SEQ ID NO: 70. In some embodiments, the therapeutic anti-CD47 antibody comprises a V_{H} that comprises SEQ ID NO: 71 and a V_{L} that comprises SEQ ID NO: 72. In some embodiments, the therapeutic anti-CD47 antibody competes for human CD47 binding against a second anti-CD47 antibody comprising an HCDR1, an HCDR2 and an HCDR3 as set forth in a V_{H} that comprises SEQ ID NO: 73 and an LCDR1, an LCDR2 and an LCDR3 as set forth in a V_{L} that comprises SEQ ID NO: 74. In some embodiments, the therapeutic anti-CD47 antibody comprises a V_{H} that comprises SEQ ID NO: 75 and a V_{L} that comprises SEQ ID NO: 76. In some embodiments, the therapeutic anti-CD47 antibody comprises a V_{H} that comprises SEQ ID NO: 77 and a V_{L} that comprises SEQ ID NO: 78. In some embodiments, the therapeutic anti-CD47 antibody comprises a V_{H} that comprises SEQ ID NO: 79 a V_{L} that comprises SEQ ID NO: 80. SEQ ID NOs: 1-80 are shown in FIG. 4. In some embodiments, the therapeutic anti-CD47 comprises a heavy chain comprising the amino acid sequence of SEQ ID NO: 216 and a light chain comprising the amino acid sequence of SEQ ID NO: 218. In some embodiments, the therapeutic anti-CD47 comprises a heavy chain comprising the amino acid sequence of SEQ ID NO: 217 and a light chain comprising the amino acid sequence of SEQ ID NO: 218.

Exemplary Methods

In some embodiments, provided is a method of reducing interference of a therapeutic anti-CD47 antibody in a serological assay using a blood sample from a subject under treatment of the therapeutic anti-CD47 antibody that comprises a human IgG4 Fc domain (or a variant thereof comprising an S228P substitution, wherein amino acid numbering is according to the EU index), the method comprising conducting a serological assay on the blood sample using an anti-human globulin (AHG) reagent that does not recognize or bind an human IgG4 antibody Fc region, e.g., ANTI-IgG (MURINE MONOCLONAL)(GREEN OR UNCOLORED) GAMMA-CLONE® commercially available from Immucor (Catalog #0409203 and 0409210). In some embodiments, the serological assay is performed at room temperature (e.g., between 17° C.-25° C.). In some embodiments, the method comprises (such as further comprises) adding an enhancer (e.g., low ionic strength saline (LISS), polyethylene glycol (PEG), saline, and albumin) to the blood sample prior to conducting the serological assay. In some embodiments, the enhancer is LISS or PEG. In some embodiments, the enhancer is added to the blood sample prior to the addition of the anti-idiotypic antibody and/or prior to conducting the serological assay, and/or prior to the agglutination step of the serological assay (e.g., prior to the addition of an agglutination agent, such as anti-human globulin (AHG), to the blood sample). In some embodiments, the blood sample is treated with EDTA. In some embodiments, the serological assay is, e.g., a direct antiglobulin test (DAT), indirect antiglobulin test (IAT), ABO test, Rh(D) blood typing test, blood cross matching, or a coombs test. In some embodiments, the method comprises (such as further comprises) comprises transfusing donor blood to the subject, wherein the donor blood is determined to be compatible with the subject, according to the results of the serological assay.

In some embodiments, the subject has been administered with at least one dose a therapeutic anti-CD47 antibody (e.g., for the treatment of an CD47-associated disease or disorder) within about any one of, e.g., 5, 10, 15, 30, 45, or 60 minutes, 1, 2, 3, 4, 5, 6, 7, 8, 9, 10, 11, 12, 13, 14, 15, 16, 17, 18, 19, 20, 21, 22, 23, or 24 hours, 1, 2, 3, 4, 5, 6, or 7 days, 1, 2, 3, 4, 5, 6, 7, or 8 weeks, or 1, 2, 3, 4, 5, or 6 or 12 months. In some embodiments, the subject has cancer. In some embodiments, the cancer is a hematological cancer. In some embodiments, the hematological cancer is non-Hodgkin lymphoma (NHL), follicular lymphoma (FL), diffuse large B-cell lymphoma (DLBCL), mantle cell lymphoma (MCL), acute myeloid leukemia (AML), chronic myeloid leukemia (CML), acute lymphoblastic leukemia (ALL) or chronic lymphoblastic leukemia (CLL). In some embodiments, the cancer is solid tumor (such as lung cancer, ovarian cancer, colorectal cancer, pancreatic cancer, sarcoma cancer, head and neck cancer, gastric cancer, renal cancer, or skin cancer, etc.). In some embodiments, the cancer is a relapsed cancer (e.g., a cancer that has relapsed or recurred during or following a prior treatment for the cancer) and/or refractory cancer (e.g., a cancer that is refractory or not responsive to a prior treatment for cancer). In some embodiments, the subject is under treatment with the therapeutic anti-CD47 antibody as a single agent. In some embodiments, the subject is under treatment with the therapeutic anti-CD47 antibody in combination with at least one additional anti-cancer agent (e.g., chemotherapeutic agent, therapeutic antibody, etc.

In some embodiments, the therapeutic anti-CD47 antibody comprises a V_{H} domain that comprises an HCDR1 comprising RAWIIN (SEQ ID NO: 81); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the CDRs of the therapeutic anti-CD47 antibody are defined according to the Kabat numbering system. In some embodiments, the therapeutic anti-CD47 antibody comprises a VH that comprises SEQ ID NO: 79 a V_{L} that comprises SEQ ID NO: 80. In some embodiments, the therapeutic anti-CD47 antibody comprises a VH that comprises SEQ ID NO: 79 a V_{L} that comprises SEQ ID NO: 80. In some embodiments, the therapeutic anti-CD47 antibody is a full-length antibody. In some embodiments, the therapeutic anti-CD47 comprises (such as further comprises) a human IgG4 Fc region or a variant thereof comprising an S228P substitution (wherein amino numbering is according to the EU index). In some embodiments, the therapeutic anti-CD47 comprises a heavy chain comprising the amino acid sequence of SEQ ID NO: 216 and a light chain comprising the amino acid sequence of SEQ ID NO: 218. In some embodiments, the therapeutic anti-CD47 comprises a heavy chain comprising the amino acid sequence of SEQ ID NO: 217 and a light chain comprising the amino acid sequence of SEQ ID NO: 218.

### Serological Assays for Pre-Transfusion Testing

Pre-transfusion testing is performed to ensure that the blood product intended for transfusion is compatible with the blood of the subject (i.e., the recipient of the transfusion). Pre-transfusion testing encompasses the serological assays that are used to confirm ABO compatibility between donor blood and recipient blood, as well as those that are used to detect most clinically significant RBC/platelet alloantibodies that react with antigens on donor RBCs and/or donor platelets (ref. Technical Manual, 18th ed, AABB, Bethesda, MD, 2014). Other exemplary blood group antigens for which serological assays are performed to determine donor/recipient transfusion compatibility include, without limitation, e.g., Kell blood group antigens, Duffy blood group antigens, Knops blood group antigens, Cartwright blood group antigens, Scianna blood group antigens, Indian blood group antigens, Rhesus blood group antigens, Dombrock blood group antigens, Landsteiner-Wiener blood group antigens, and VEL blood group antigens. The methods provided herein reduce or prevent drug interference (e.g., interference by a drug comprising (i) an antibody Fc region and (ii) a moiety that binds to human CD47) in a number of serological assays known in the art. Exemplary serological assays in which the methods can be used include (but are not limited to) those described in further detail below.

Typically, serological assays are performed using samples comprising, e.g., non-hemolyzed blood, plasma (e.g., a plasma sample that has been anticoagulated in EDTA), clotted blood, or serum from a subject who is in need of the transfusion (e.g., a subject who is under treatment with a therapeutic anti-CD47 antibody). In general, the subject's ABO group and Rh type are determined first. Next, an antibody screening method is used to detect any clinically significant unexpected non-ABO blood group antibodies that may be present in the subject's plasma. If the screening test reveals the presence of such an antibody, the specificity of that antibody is determined using an antibody identification panel. After the specificity of the antibody is identified, donor units of the appropriate ABO group and Rh type are screened for the corresponding antigen. Units that are negative for that antigen are cross-matched with the subject who is in need of the transfusion to ensure compatibility.

Serological assays can be performed in a tube, on a slide, on a gel column or in microtiter well plates, and hemolysis and agglutination are signals that indicate a positive (incompatible) test result. Agglutination, a reaction reflecting linkage of adjacent RBCs that are coated with antibody, can be scored macroscopically and/or microscopically and on scale from 0-4+ in the most commonly used tube methods. A score of zero indicates no reactivity and is characterized by smooth and easily dispersed cells. A score of 4+ indicates strong reactivity and is characterized by one solid agglutinate that is not easily dispersed. Scores of 1+, 2+, or 3+ indicate intermediate levels of reactivity, characterized by gradually increasing size of agglutinates with higher scores. Similar principles of agglutination scoring can be applied when the serological tests are conducted using gel columns with anti IgG antibody in the column (gel card) or microtiter well plates with bound red blood cell antigens (solid phase). Various techniques are currently available for the detection of antibody-RBC antigen interaction with varying sensitivities. In some embodiments, serological assays are performed manually. In some embodiments, serological assays are performed via automated machine.

For example, immediate-spin (IS) (also known as “immediate spin crossmatch”) is an assay that entails mixing, e.g., reagent plasma/antisera (i.e., plasma containing antibodies against a known RBC and/or platelet surface antigen) and the subject's blood cells, immediately centrifuging the mixture for about 15-30 seconds at room temperature or at 37° C., and visually examining the tube for direct agglutination. Direct agglutination indicates that there is a strong interaction between an antibody in the plasma and an RBC surface antigen. Alternatively, the subject's plasma and reagent RBC (i.e., RBC that are known to express a particular cell surface antigen, or group of cell surface antigens) and/or regent platelets (i.e., platelets that are known to express a particular cell surface antigen, or group of cell surface antigens) can be mixed, centrifuged, and assessed visually for direct agglutination.

Anti-human globulins (AHGs) are used to detect antibody-bound RBC that do not produce direct agglutination. AHG are secondary anti-human globulin antibodies that have been produced in another species. AHG reagents can be specific for a single class of human Ig (such as IgG), or polyspecific, i.e., capable of binding to multiple human Ig classes (e.g., IgG, IgM, IgA) and to complement. In some embodiments, the AHG reagent does not bind to a human IgG4 Fc region. AHG sera may be used in a direct antiglobulin test (DAT) and/or in an indirect antiglobulin test (IAT). The DAT demonstrates in vivo sensitization of red cells and is performed by directly testing a sample of washed patient red cells with AHG. An IAT demonstrates in vitro reactions between red cells and antibodies. In an IAT, serum (or plasma) is incubated with red cells, which are then washed to remove unbound globulins. The presence of agglutination with the addition of AHG indicates antibody binding to a specific red cell antigen. Some methods involve addition of potentiator reagents (enhancement) such as saline, albumin, low ionic strength saline (LISS), or polyethylene glycol (PEG), and the samples are then incubated at 37° C. for 10-60 minutes prior to the AHG test.

ABO typing involves testing the recipient's red blood cells for the presence of A and B antigens using anti-A and anti-B antisera (forward grouping). Testing of the recipient plasma for the presence of anti-A and anti-B using known Type A and Type B red blood cells (reverse grouping) is also part of routine ABO blood group testing.

The Rh (D) type of the transfusion recipient is determined by testing recipient red blood cells with anti-D. ABO grouping is typically tested using immediate spin (IS).

Alloantibodies to antigens that are not present on an individual's red blood cells may develop in anyone who has been exposed to foreign red blood cell antigens through pregnancy or transfusion. To detect antibodies to non-group A or B antigens, a sample of the patient's plasma or serum is tested against selected commercial Type O red blood cells that express the majority of clinically significant antigens, other than A and B.

In cases of positive antibody screening, further serological testing is conducted with an expanded panel of commercial Type O reagent RBCs for the identification of clinically significant antibodies is required. Then, once the specificity of the antibody is known, donor units must be screened for the corresponding antigen to select those units that lack the antigen.

Antigen typing (phenotyping) of the recipient red blood cells may also be performed to determination of which red blood cell antibodies an individual is likely to develop. Serological assay for RBC phenotyping involves mixing recipient cells with commercial reagent anti-sera containing specific antibodies.

An IAT without and with enhancement (e.g. saline, LISS, PEG) is used in antibody detection and antibody identification.

“Crossmatch” refers to a method of confirming compatibility between the patient's blood (plasma) and the donor red blood cells. The crossmatch is meant primarily to detect and prevent ABO incompatibility. A serological crossmatch assay (either IS crossmatch or AHG phase crossmatch) involves the direct mixing of donor red blood cells with recipient plasma and scores for hemolysis and agglutination following immediate-spin method or AHG test.

Methods of Transfusing Donor Blood to a Subject Under Treatment with a Therapeutic Anti-CD47 Antibody

Also provided herein are methods of transfusing donor blood to a subject under treatment with a therapeutic anti-CD47 antibody. In some embodiments, the method comprises performing a method for mitigating the interference in a pre-transfusion serological assay caused by a therapeutic anti-CD47 antibody described herein. In some embodiments, the method further comprises performing a serological assay (e.g., a serological assay described above). In some embodiments, the method further comprises transfusing donor blood to the subject, wherein the donor blood is determined to be compatible with the subject, according to the results of the serological assay.

In some embodiments, the subject under treatment with the therapeutic anti-CD47 antibody receives the transfusion within about any one of, e.g., 5, 10, 15, 30, 45, 60, 90, or 120 minutes, 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, or 96 hours, 1, 1.5, 2, 2.5, 3, 3.5, 4, 4.5, or 5 days or 1, 2, 3, 4, 5, 6 or 12 months after being administered with the therapeutic anti-CD47 antibody. In some embodiments, the transfusion is provided to the subject on a fixed schedule, e.g., any of every 1, 2, 3, 4, 5, 6, 7, 8, 9, or 10 days, every 1, 2, 3, 4, 5, 6, 7, 8, 9, or 10 weeks, or every 1, 2, 3, 4, or 5 months throughout the entire treatment cycle with the therapeutic anti-CD47 antibody.

In some embodiments, the subject has been administered with at least one dose a therapeutic anti-CD47 antibody (e.g., for the treatment of an CD47-associated disease or disorder) within about any one of, e.g., 5, 10, 15, 30, 45, or 60 minutes, 1, 2, 3, 4, 5, 6, 7, 8, 9, 10, 11, 12, 13, 14, 15, 16, 17, 18, 19, 20, 21, 22, 23, or 24 hours, 1, 2, 3, 4, 5, 6, or 7 days, 1, 2, 3, 4, 5, 6, 7, or 8 weeks, or 1, 2, 3, 4, 5, or 6 or 12 months. In some embodiments, the subject has cancer. In some embodiments, the cancer is a hematological cancer. In some embodiments, the hematological cancer is non-Hodgkin lymphoma (NHL), follicular lymphoma (FL), diffuse large B-cell lymphoma (DLBCL), or mantle cell lymphoma (MCL). In some embodiments, the cancer is solid tumor (such as lung cancer, ovarian cancer, colorectal cancer, pancreatic cancer, sarcoma cancer, head and neck cancer, gastric cancer, renal cancer, or skin cancer, etc.). In some embodiments, the cancer is a relapsed cancer (e.g., a cancer that has relapsed or recurred during or following a prior treatment for the cancer) and/or refractory cancer (e.g., a cancer that is refractory or not responsive to a prior treatment for cancer). In some embodiments, the subject is under treatment with the therapeutic anti-CD47 antibody as a single agent. In some embodiments, the subject is under treatment with the therapeutic anti-CD47 antibody in combination with at least one additional anti-cancer agent (e.g., chemotherapeutic agent, therapeutic antibody, etc.).

In some embodiments, the subject has anemia. In some embodiments, the anemia results from/is induced by administration of the therapeutic anti-CD47 antibody to the subject. In some embodiments, the subject has anemia if the subject's hemoglobin level is below about any one of 12 g/dL, 11 g/dL, 10 g/dL, 9 g/dL, or 8 g/dL. In some embodiments, the subject is transfused with donor blood (e.g., compatible donor blood) within about any one of, e.g., 30, 60, 90 minutes after the serological assay has been performed or within about any one of, e.g., 1, 2, 3, 4, 5, 6, 7, 8, 9, 10, 11, 12, 18, 24, 30, 36, 42, 48, 54, 60, 66, 72, 78, 84, 90, or 96 hours after the serological assay has been performed.

In some embodiments, the therapeutic anti-CD47 antibody comprises a V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86). In some embodiments, the CDRs of the therapeutic anti-CD47 antibody are defined according to the Kabat numbering system. In some embodiments, the therapeutic anti-CD47 antibody comprises a V_{H} that comprises SEQ ID NO: 79 a V_{L} that comprises SEQ ID NO: 80. In some embodiments, the therapeutic anti-CD47 antibody comprises a V_{H} that comprises SEQ ID NO: 79 a V_{L} that comprises SEQ ID NO: 80. In some embodiments, the therapeutic anti-CD47 antibody is a full-length antibody. In some embodiments, the therapeutic anti-CD47 comprises (such as further comprises) a human IgG4 Fc region or a variant thereof comprising an S228P substitution (wherein amino numbering is according to the EU index). In some embodiments, the therapeutic anti-CD47 comprises a heavy chain comprising the amino acid sequence of SEQ ID NO: 216 and a light chain comprising the amino acid sequence of SEQ ID NO: 218. In some embodiments, the therapeutic anti-CD47 comprises a heavy chain comprising the amino acid sequence of SEQ ID NO: 217 and a light chain comprising the amino acid sequence of SEQ ID NO: 218.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1. Preparation of the F(ab′)2 Fragment of an Anti-CD47 Antibody for Generation of Anti-Idiotypic Antibodies

The F(ab′)₂ fragment of an anti-CD47 antibody was prepared by Papain digestion. Briefly, anti-CD47 antibody TJC4, which comprises a VH domain that comprises SEQ ID NO: 79 and a V_{L} domain that comprises SEQ ID NO: 80, was digested by incubation with pepsin (5 μg pepsin per mg antibody) for 30 min at 37° C. After digestion, the mixture was applied to a gel filtration column followed by an affinity column to recover the fraction of F(ab′)₂ fragment, and the purity of F(ab′)₂ fraction was detected by SDS-PAGE.

### Example 2. Generation of Anti-Idiotypic Antibodies that Bind to the Anti-CD47 Antibody

Anti-idiotypic antibodies were generated by hybridoma technology. Generating the anti-idiotypic antibodies entailed (1) immunizing mice with the anti-CD47 antibody fragment prepared in Example 1; (2) performing indirect ELISAs to test immune responses of the mice against the anti-CD47 antibody fragment; (3) obtaining antibodies from the mice found to have the highest titers against the anti-CD47 antibody fragment for hybridoma fusion, and (4) cloning and characterizing the antibodies produced by each hybridoma. The scFv/Fab forms of the anti-CD47 antibody were used to immunize mice in the presence of isotype matching control antibodies for counter-selection.

Briefly, six mice (balb/c) were immunized with F(ab′)2 fragments of an anti-CD47 antibody TJC4. After 3 boosts, serum titers were evaluated by standard enzyme-linked immunosorbent assay (ELISA) to identify mice with positive serum titers to the anti-CD47 F(ab′)₂ fragment. B cells from two mice having the highest antibody titers against the anti-CD47 F(ab′)₂ fragment were then fused with mouse myeloma cells via electro fusion. The fusion efficiency was approximately 1 hybridoma/3000 B cells. All fused cells from each cell fusion were plated into 96-well plates. After several days, hybridoma supernatants were harvested and screened against the anti-CD47 F(ab′)2 fragment by indirect ELISA for anti-CD47 antibody specific anti-idiotype antibody production. Antibodies exhibiting a strong assay signal as compared to the associated reference signal, were selected as candidates for further characterization and development.

Three positive primary clones selected were subcloned by limiting dilution to ensure that the subclones were derived from a single parental cell. The subclones were then inoculated and cultured. The anti-idiotypic antibodies were purified from the harvest culture supernatant by Protein A affinity chromatography and were screened by indirect ELISA and antigen blocking assay. The purified antibodies were dialyzed into PBS buffer for storage. The products were certified by quality control, including indirect ELISA, IC50, purity test by SDS-PAGE, and concentration determination by absorption at OD280 nm.

### Example 3. Cloning and Sequence Analysis of Anti-Idiotypic Antibodies

Nucleic acid sequences of the anti-idiotypic antibodies were cloned for recombinant antibody production as follows. cDNAs encoding antibody variable domains were amplified from total RNA isolated from each hybridoma cell via PCR. The nucleic acid sequences of antibody heavy chain variable domains (V_{H}) and light chain variable domains (V_{L}) were amplified according to the standard operating procedure (SOP) of rapid amplification of cDNA ends (RACE) by GenScript. Amplified antibody fragments were each cloned into a standard cloning vector.

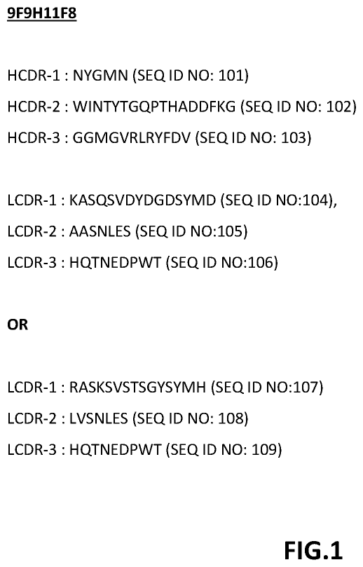

The amino acid sequences of the V_{H} and V_{L} for the monoclonal anti-idiotype antibodies were determined as shown in FIGS. 1, 2, 5A, and 5B.

### Example 4. Characterization of Anti-idiotype Antibodies Binding to CD47 Antibody

The binding of the anti-idiotypic antibodies generated in Examples 2 and 3 to anti-CD47 TJC4 antibody was characterized by indirect ELISA assay, and the IC₅₀ of the anti-idiotypic antibodies was used as a measure of their potential effectiveness in reducing interference by a therapeutic anti-CD47 antibody in a serological assay. Lower IC₅₀ values indicated stronger blocking ability. The lowest IC₅₀ value for any hybridoma clone was determined to be 381.5 ng/ml for anti-idiotypic antibody 9F9H11H8. The IC₅₀ value for 9F9H11H8, and, therefore, the blocking ability of 9F9H11H8, was better than those of 37F8C4G12 and 34D8H11B5 (i.e., anti-idiotypic antibodies produced by other hybridoma clones).

### Example 5. Competitive Binding Assay Shows that 9F9H11H8 Prevents Anti-CD47 Antibody TJC4 from Binding to CD47 on Red Blood Cells (RBCs)

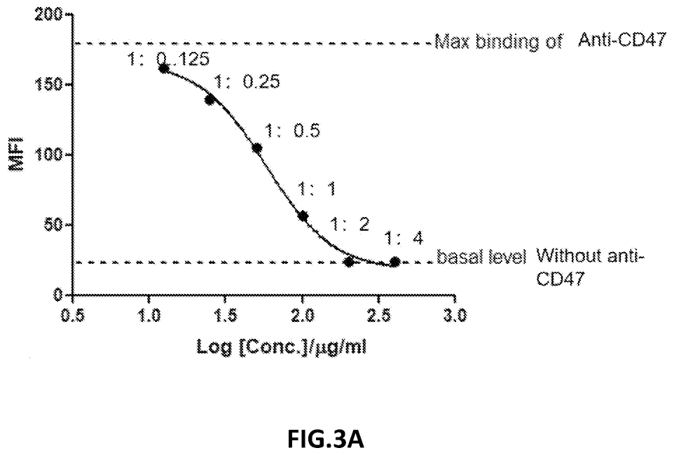

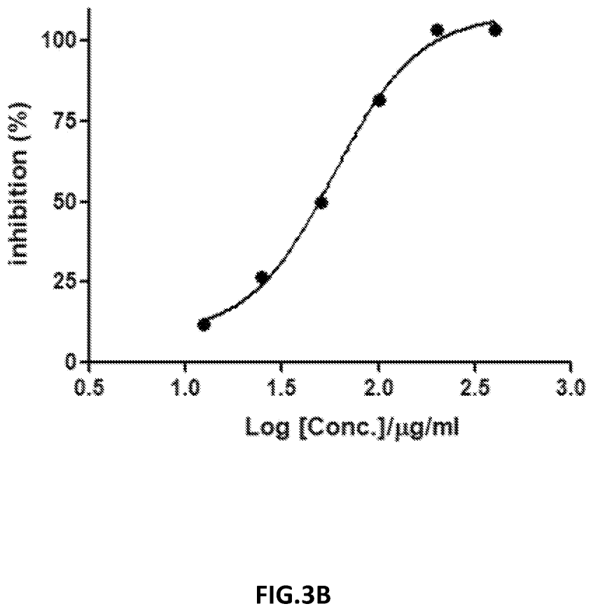

A flow cytometry-based competitive bindings analysis was performed as follows. Anti-CD47 antibody TJC4 was incubated with anti-idiotypic antibody 9F9H11H8, which comprises a V_{H} domain comprising SEQ ID NO: 110 and a V_{L} domain comprising SEQ ID NO: 113, for 30 minutes at room temperature at the following ratios of anti-CD47 antibody: anti-idiotypic antibody: 1:0, 1:0.125, 1:0.25, 1:0.5, 1:1, 1:2, 1:4, and 0:1. Next, 50 μL of a3% red blood cell (RBC) suspension was added to the antibody mixture and incubating further for another 30 minutes at room temperature. Anti-human IgG4 conjugated with allophycocyanin (APC) fluorescent dye was used as a secondary antibody to detect the binding of the anti-CD47 antibody to CD47 on the surface of the RBCs. The fluorescence intensity of each sample was measured. Mean fluorescent intensities (MFI) for each sample were calculated from the histograms and used to plot the binding curves shown in FIG. 3A. Percent inhibition of anti-CD47 binding to RBCs at each of ratio of anti-CD47 antibody: anti-idiotypic antibody were determined (see FIG. 3B).

FIG. 3A is a schematic diagram of the binding of anti-CD47 antibody TJC4 to CD47 on the surface of RBCs, while in the presence of anti-idiotypic antibody 9F9H11H8 (which comprises a V_{H} domain comprising SEQ ID NO: 110 and a V_{L} domain comprising SEQ ID NO: 113) at specified ratios of anti-CD47 antibody to anti-idiotypic antibody. The fluorescence intensity of 200 μg/ml anti-CD47 antibody bound to RBC, wherein the binding of the anti-CD47 antibody to RBC was detected by labeled secondary antibody, was used as the maximum fluorescence intensity. The fluorescence intensity of RBC itself (i.e., without any anti-CD47 antibody) was used as baseline. If anti-CD47 antibody binding to RBCs were inhibited by the anti-idiotypic antibody, the fluorescence resulting from the binding of the labeled anti-IgG4 secondary antibody to the anti-CD47 antibody bound to the RBCs would not be detected by flow cytometry. The results in FIGS. 3A and 3B show that fluorescence intensity decreases gradually with increasing amounts of anti-idiotypic antibody. FIG. 3A shows that anti-idiotypic antibody 9F9H11H8 completely inhibits the binding of anti-CD47 antibody TJC4 to RBCs at an anti-CD47 antibody to anti-idiotypic antibody ratio of 1:2. FIG. 3B shows the percentage of inhibition of the binding of the anti-CD47 antibody to RBCs at different anti-CD47 antibody to anti-idiotypic antibody ratios.

### Example 6. Mitigation of Interference by an Anti-CD47 Antibody in Pre-Transfusion Tests Using an Anti-Idiotypic Antibody

### Reagent:

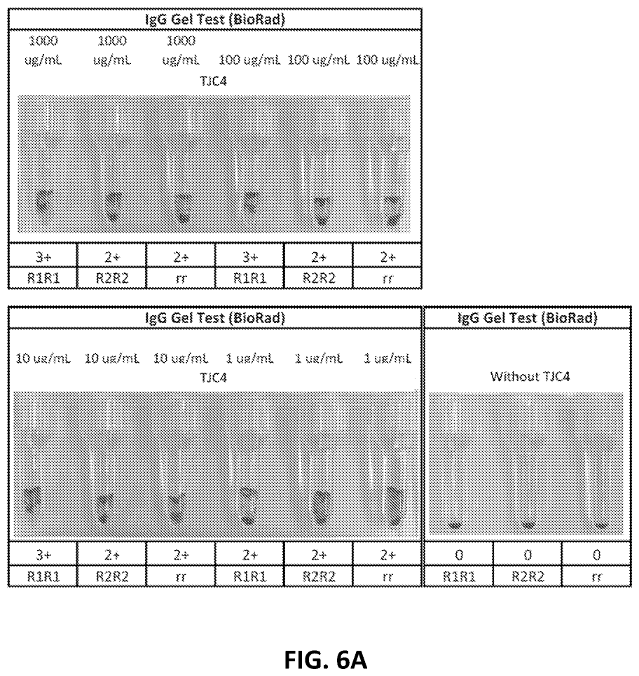

A blood sample donated by a healthy volunteer was divided into 5 vials. Each vial was spiked with 0 μg/mL (control), 1 μg/mL, 10 μg/mL, 100 μg/mL, or 1000 μg/mL of anti-CD47 antibody TJC4 and incubated at 37° C. for 15 min. 25 μL of plasma isolated from each vial was mixed with 1% RBC suspension (Bio-Rad platform) with R₁R₁, R₂R₂, or rr phenotype, respectively. The mixtures was incubated at 37° C. for 15 min and then centrifuged at 900 rpm for 10 min for IAT testing. As shown in FIG. 6A, the presence of anti-CD47 antibody TJC4 at 1 μg/mL, 10 μg/mL, 100 μg/mL, and 1000 μg/mL caused moderate to strong (2+ to 3+) interference in IAT assay in all phenotypes including R₁R₁, R₂R₂ and rr in Bio-Rad Platform.

To assess whether the anti-idiotypic antibody 9F9H11H8 (which comprises a VH domain comprising SEQ ID NO: 110 and a VL domain comprising SEQ ID NO: 113) could mitigate the interference in the IAT assay caused by TJC4, 100 μL plasma was spiked with 200 μg/ml of TJC4 was mixed with 70 μL 9F9H11H8 (i.e., a total of 0.1 mg 9F9H11H8, achieving a 5:1 w/w ratio of 9F9H11H8:TJC4) or 40 μL 9F9H11H8 (i.e., a total of 0.06 mg 9F9H11H8, achieving a 2:1 w/w ratio of 9F9H11H8:TJC4) and incubated at 37° C. for 1 hour. Next, 25 μL of each mixture was mixed with 1% RBC suspension (Bio-Rad platform) with R₁R₁, R₂R₂, or rr phenotype, respectively. Plasma without TJC4 treatment was used as negative control.

It was found that the anti-idiotypic 9F9H11H8 significantly mitigated the interference in the IAT assay caused by the presence of TJC4 in the blood sample. Such result suggests that 9F9H11H8 can mitigate interference in serological assays that use blood samples from subjects under treatment with a therapeutic anti-CD47 antibody (such as TJC4).

### Example 7. Mitigation of Anti-CD47 Antibody Interference in IAT Using Immucor Gamma Clone Anti-IgG

a) Interference Resulted from Treatment of Anti-CD47 Antibody in IAT Assay.

A blood sample donated by a healthy volunteer was divided into separate vials. The vials were spiked with anti-CD47 antibody TJC4 at concentration of 1 μg/ml, 10 μg/ml, 100 μg/ml or 1000 μg/ml, respectively at 37° C. for 1 hour. Plasma was isolated from each of the vials.

50 μl of each plasma sample was incubated with 100 μl commercial red blood cells with a phenotype of R₁R₁, R₂R₂, or rr for 15 minutes. Control plasma samples, to which commercial red blood cells were not added, were prepared in parallel. Interference of the anti-CD47 antibody (i.e., binding of anti-CD47 antibody TJC4 to CD47 on the surface of the red blood cells) in each plasma sample was measured, and the results are shown in Table 1 below. The presence of ani-CD47 antibody TJC4 resulted in interference from panreaction at 10 and 100 μg/ml. However, the interference was not observed under high concentration of TJC4 such as 1000 μg/ml, which may be resulted from prozone effect by the antibody.

TABLE 1
Interference in IAT resulting from presence
of anti-CD47 antibody in a plasma sample*

TJC4 (μg/ml)
Concentration of TJC4 in plasma sample

| | 1 μg/ml | 10 μg/ml | 100 μg/ml | 1000 μg/ml |
|---|---|---|---|---|
| R₁R₁ | 0 | 1+ | 1+ | 0* |
| R₂R₂ | 0 | 1+ | 1+ | 0* |
| rr | 0 | 1+ | 1+ | 0* |

0, micro+, 1+, 2+, 3+ and 4+ illustrate different levels of positive reactivity, i.e. interference by anti-CD47 antibody, determined by microscope.
*indicates prozone effect that resulted from high concentration of anti-CD47 antibody.

b) Reduction of Interference in IAT by Immucor Anti-IgG AHG, which does not Bind with Human IgG4

Plasma samples were prepared as described above in section a). 2 drops of Immucor anti-IgG, which does not bind to the human IgG4 Fc region was added to the reaction system and then centrifuged immediately. Ortho anti-IgG which binds to human IgG4 was used as a positive control. The results are shown in Table 2 and Table 3. As can be seen from Table 2 and Table 3, treatment with the anti-IgG that does not bind with human IgG4, such as Immucor anti-IgG, mitigated the interference resulted from anti-CD47 antibody in the system. In contrast, stronger interference was observed when the anti-IgG reagent that binds human IgG4 was used.

TABLE 2
Reduction of Interference in IAT using anti-IgG that
does not bind with human IgG4 (Immucor anti-IgG)

| | Saline 37° C. | | | |
|---|---|---|---|---|
| | TJC4 (μg/ml) | | | |
| | Concentration of TJC4 in plasma sample | | | |
| | 1 μg/ml | 10 μg/ml | 100 μg/ml | 1000 μg/ml |
| | (without/with | (without/with | (without/with | (without/with |
| | Immucor | Immucor | Immucor | Immucor |
| | anti-IgG) | anti-IgG) | anti-IgG) | anti-IgG) |
| R₁R₁ | 0/0 | 2+/0 | 1+/0 | 0*/0 |
| R₂R₂ | 0/0 | 2+/0 | 1+/0 | 0*/0 |
| rr | 0/0 | 1+/0 | 2+/0 | 0*/0 |

0, micro+, 1+, 2+, 3+ and 4+ illustrate different levels of positive reactivity, i.e. interference by anti-CD47 antibody, determined by microscope.
*indicates prozone effect that resulted from high concentration of anti-CD47 antibody.

TABLE 3
Interference in IAT using anti-IgG binding
that binds human IgG4 (Ortho anti-IgG)

| | Saline 37° C. | | | |
|---|---|---|---|---|
| | TJC4 (μg/ml) | | | |
| | Concentration of TJC4 in plasma sample | | | |
| | 1 μg/ml | 10 μg/ml | 100 μg/ml | 1000 μg/ml |
| | (without/with | (without/with | (without/with | (without/with |
| | Immucor | Immucor | Immucor | Immucor |
| | anti-IgG) | anti-IgG) | anti-IgG) | anti-IgG) |
| R₁R₁ | 0/4+ | 2+/4+ | 2+/4+ | 0*/4+ |
| R₂R₂ | 0/4+ | 2+/4+ | 2+/4+ | 0*/4+ |
| rr | 0/3+ | 2+/4+ | 2+/4+ | 0*/4+ |

0, micro+, 1+, 2+, 3+ and 4+ illustrate different levels of positive reactivity, i.e. interference by anti-CD47 antibody, determined by microscope.
*indicates prozone effect that resulted from high concentration of anti-CD47 antibody.

c) Effect of Enhancer on IAT Using Anti-IgG that does not Bind with Human IgG4* (Immucor Anti-IgG)

Plasma samples were prepared as described above in section a). 100 μg/ml of enhancer (i.e., LISS or PEG) was added to the system and incubated for 15 minutes at 37° C. Then). 2 drops of Immucor anti-IgG, which does not bind to the human IgG4 Fc region was added to the reaction system and then centrifuged immediately. As shown in Table 4 and Table 5, robust interference induced by anti-CD47 antibody was observed after adding the enhancer. Addition of an anti-IgG that does not bind with human IgG4, however, mitigated the interference to a micro level.

TABLE 4
Reduction of interference in IAT using anti-IgG which does not
bind with human IgG4 (Immucor anti-IgG), with enhancer LISS

| | LISS enhancer | | | |
|---|---|---|---|---|
| | TJC4 (μg/ml) | | | |
| | Concentration of TJC4 in plasma sample | | | |
| | 1 μg/ml | 10 μg/ml | 100 μg/ml | 1000 μg/ml |
| | (without/with | (without/with | (without/with | (without/with |
| | Immucor | Immucor | Immucor | Immucor |
| | anti-IgG) | anti-IgG) | anti-IgG) | anti-IgG) |
| R₁R₁ | 2+/0 | 3+/Micro+ | 3+/Micro+ | 2+/0 |
| R₂R₂ | 1+/0 | 3+/Micro+ | 3+/Micro+ | 2+/Micro+ |
| rr | 1+/0 | 3+/Micro+ | 3+/Micro+ | 1+/Micro+ |

0, micro+, 1+, 2+, 3+ and 4+ illustrate different levels of positive reactivity, i.e. interference by anti-CD47 antibody, determined by microscope.
*indicates prozone effect that resulted from high concentration of anti-CD47 antibody.

TABLE 5
Reduction of interference in IAT using anti-IgG which does not
bind with human IgG4 (Immucor anti-IgG), with enhancer PEG

| | PEG enhancer | | | |
|---|---|---|---|---|
| | TJC4 (μg/ml) | | | |
| | Concentration of TJC4 in plasma sample | | | |
| | 1 μg/ml | 10 μg/ml | 100 μg/ml | 1000 μg/ml |
| | (without/with | (without/with | (without/with | (without/with |
| | Immucor | Immucor | Immucor | Immucor |
| | anti-IgG) | anti-IgG) | anti-IgG) | anti-IgG) |
| R₁R₁ | 0 | Micro+ | Micro+ | 0* |
| R₂R₂ | 0 | Micro+ | Micro+ | 0* |
| rr | 0 | Micro+ | Micro+ | 0* |

0, micro+, 1+, 2+, 3+ and 4+ illustrate different levels of positive reactivity, i.e. interference by anti-CD47 antibody, determined by microscope.
*indicates prozone effect that resulted from high concentration of anti-CD47 antibody.

From above results, it can be seen that the presence of an anti-CD47 antibody such as TJC4 in a blood sample may cause plasma interference in serological assay. After the treatment with Immucor anti-IgG, which does not bind to human IgG4, interference in IAT was reduced. However, robust interference was observed by using Ortho anti-IgG, which does bind to human IgG4.

When LISS or PEG was added to the system, robust interference induced by anti-CD47 antibody was observed. Treatment with an anti-IgG that does not bind to human IgG4 reduced the robust interference to micro level.

Above results suggests use of the anti-IgG that does not bind to human IgG4 in mitigating interference in serological assay for patient under treatment with a therapeutic anti-CD47 antibody (such as TJC4).

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

1. A method of reducing interference of a therapeutic anti-CD47 antibody in a serological assay using a blood sample from a subject under treatment of the therapeutic anti-CD47 antibody, comprising:
adding an anti-idiotypic antibody specifically recognizing an antigen binding portion of the therapeutic anti-CD47 antibody to the blood sample before conducting the serological assay,
wherein the therapeutic anti-CD47 antibody competes for human CD47 binding against an anti-CD47 antibody that comprises an HCDR1, an HCDR2 and an HCDR3 as set forth in a light chain variable domain (V_{H}) comprising SEQ ID NO:79 and an LCDR1, an LCDR2 and an LCDR3 as set forth in a light chain variable domain (V_{L}) comprising SEQ ID NO: 80.

2. A method of conducting a serological assay using a blood sample of an individual who is under treatment with a therapeutic anti-CD47 antibody, the method comprising:
(a) adding an anti-idiotypic antibody specifically recognizing an antigen binding portion of the therapeutic anti-CD47 antibody to the blood sample; and
(b) performing the serological assay on the blood sample,
wherein the therapeutic anti-CD47 antibody competes for human CD47 binding against an anti-CD47 antibody that comprises an HCDR1, an HCDR2 and an HCDR3 as set forth in a heavy chain variable domain (V_{H}) comprising SEQ ID NO:79 and an LCDR1, an LCDR2 and an LCDR3 as set forth a light chain variable domain (V_{L}) comprising SEQ ID NO:80, and
wherein the addition of the anti-idiotypic antibody reduces interference of the therapeutic anti-CD47 antibody in the serological assay.

3. The method of claim 1 or 2, wherein the therapeutic anti-CD47 antibody comprises: a V_{H} that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83); and a V_{L} that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86).

4. The method of claim 3, wherein the therapeutic anti-CD47 antibody comprises a V_{H} that comprises SEQ ID NO: 79 and a V_{L} that comprises SEQ ID NO: 80.

5. The method of any one of claims 1-4, wherein the therapeutic anti-CD47 antibody comprises a human IgG4 Fc region or a variant thereof that comprises an S228P substitution, wherein amino acid numbering is according to the EU index.

6. The method of any one of claims 1-5, wherein the anti-idiotypic antibody comprises:
(a) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106);
(b) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109);
(c) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising AASNLES (SEQ ID NO: 105); and an LCDR3 comprising QQTHEDPWT (SEQ ID NO: 220);
(d) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising QQTHEDPWT (SEQ ID NO: 220);
(e) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising AASNLES (SEQ ID NO: 105); and an LCDR3 comprising QQTHEDPWT (SEQ ID NO: 220);
(f) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising LVSNLES (SEQ ID NO: 108); and an LCDR3 comprising QQTHEDPWT (SEQ ID NO: 220);
(g) a V_{H} domain that comprises an HCDR1 comprising DYNMN (SEQ ID NO: 100); an HCDR2 comprising YVDPYYGDTRYNQNFKG (SEQ ID NO: 235); and an HCDR3 comprising SETPRAMDY (SEQ ID NO: 236); and a light chain variable (V_{L}) domain that comprises an LCDR1 comprising RASQSISDYLH (SEQ ID NO: 237); an LCDR2 comprising YASQSIS (SEQ ID NO: 238); and an LCDR3 comprising QNGHSLPLT (SEQ ID NO: 239).

7. The method of claim 6, wherein the anti-idiotypic antibody comprises
(a) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106; or
(b) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109).

8. The method of claim 6, wherein the anti-idiotypic antibody comprises:
(a) a V_{H} comprising SEQ ID NO: 110 and a V_{L} comprising SEQ ID NO: 111;
(b) a V_{H} comprising SEQ ID NO: 110 and a V_{L} comprising SEQ ID NO: 112;
(c) a V_{H} comprising SEQ ID NO: 110 and a V_{L} comprising SEQ ID NO: 113;
(d) a V_{H} comprising SEQ ID NO: 110 and a V_{L} comprising SEQ ID NO: 114;
(e) a V_{H} comprising SEQ ID NO: 95 and a V_{L} comprising SEQ ID NO: 87;
(f) a V_{H} comprising SEQ ID NO: 95 and a V_{L} comprising SEQ ID NO: 88;
(g) a V_{H} comprising SEQ ID NO: 95 and a V_{L} comprising SEQ ID NO: 89;
(h) a V_{H} comprising SEQ ID NO: 95 and a V_{L} comprising SEQ ID NO: 90;
(i) a V_{H} comprising SEQ ID NO: 95 and a V_{L} comprising SEQ ID NO: 91;
(j) a V_{H} comprising SEQ ID NO: 95 and a V_{L} comprising SEQ ID NO: 92; or
(k) a V_{H} comprising SEQ ID NO: 93 and a V_{L} comprising SEQ ID NO: 94.

9. The method of claim 8, wherein the anti-idiotypic antibody comprises:
(a) a V_{H} comprising SEQ ID NO: 110 and a V_{L} comprising SEQ ID NO: 111;
(b) a V_{H} comprising SEQ ID NO: 110 and a V_{L} comprising SEQ ID NO: 112;
(c) a V_{H} comprising SEQ ID NO: 110 and a V_{L} comprising SEQ ID NO: 113; or
(d) a V_{H} comprising SEQ ID NO: 110 and a V_{L} comprising SEQ ID NO: 114.

10. The method of any one of claims 1-9, wherein the binding affinity of the anti-idiotypic antibody to the therapeutic anti-CD47 antibody is higher than the binding affinity of human CD47 on the surface of red blood cells to the therapeutic anti-CD47 antibody.

11. The method of any one of claims 1-10, wherein the anti-idiotypic antibody is added to the blood sample in an excess amount relative to the amount of the therapeutic anti-CD47 antibody in the blood sample.

12. The method of any one of claims 1-11, wherein the anti-idiotypic antibody is added to the blood sample in amount sufficient to achieve a molar ratio of between about 1:1 and about 3:1 of anti-idiotypic antibody relative to therapeutic anti-CD47 antibody in the blood sample.

13. The method of claim 12, wherein the anti-idiotypic antibody is added to the blood sample in an amount sufficient to achieve a molar ratio of about 2:1 of anti-idiotypic antibody relative to therapeutic anti-CD47 antibody in the blood sample.

14. The method of any one of claims 1-13, wherein the serological assay is selected from the group consisting of direct antiglobulin test (DAT), indirect antiglobulin test (IAT), ABO test, Rh(D) blood typing test, blood cross matching, and coombs test.

15. The method of claim 14, wherein the serological assay is an indirect antiglobulin test (IAT).

16. The method of claim 14, wherein the serological assay is a direct antiglobulin test (DAT).

17. The method of claim 15, wherein the serological assay is an eluate test performed after a DAT assay.

18. The method of any one of claims 1-17, wherein the concentration of the therapeutic anti-CD47 antibody in the blood sample is between about 20 μg/ml and about 1500 μg/ml.

19. The method of any one of claims 1-18, wherein the method comprises incubating the blood sample and the anti-idiotypic antibody for at least about 15 minutes prior to conducting the serological assay.

20. The method of claim 19, wherein the incubation is carried out at 37° C.

21. A method of conducting a serological assay in a blood sample of an individual who under treatment with a therapeutic anti-CD47 antibody comprising an IgG4 Fc domain, the method comprising: conducting a direct antiglobulin testing (DAT) or indirect antiglobulin testing (IAT) on the blood sample using an anti-human globulin (AHG) anti-IgG that does not recognize a human IgG4 antibody Fc region.

22. The method of claim 21, wherein the therapeutic anti-CD47 antibody comprises:
(a) a V_{H} domain that comprises an HCDR1 comprising SYAMS (SEQ ID NO: 115); an HCDR2 comprising AISGSGGSTYYADSVKG (SEQ ID NO: 116); and an HCDR3 comprising YSIGRHTFDH (SEQ ID NO: 117) and a (V_{L}) domain that comprises an LCDR1 comprising TRSSGGIASNFVQ (SEQ ID NO: 118); an LCDR2 comprising RDNQRPS (SEQ ID NO: 119); and an LCDR3 comprising QSYDDHNHWV (SEQ ID NO: 120);
(b) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(c) a V_{H} domain that comprises an HCDR1 comprising GYAMT (SEQ ID NO: 124); an HCDR2 comprising AITSTGGRTYYADSVKG (SEQ ID NO: 125); and an HCDR3 comprising ESNFRAFDI (SEQ ID NO: 126) and a (V_{L}) domain that comprises an LCDR1 comprising RSSQSLLHSNGYNYLD (SEQ ID NO: 127); an LCDR2 comprising LNSNRAS (SEQ ID NO: 128); and an LCDR3 comprising MQALQIPPT (SEQ ID NO: 129);
(d) a V_{H} domain that comprises an HCDR1 comprising DAWMT (SEQ ID NO: 130); an HCDR2 comprising VIYSGGSTYYADSVKG (SEQ ID NO: 131); and an HCDR3 comprising GARGHPGQDY (SEQ ID NO: 132) and a (V_{L}) domain that comprises an LCDR1 comprising TRSSGTIASNFVQ (SEQ ID NO: 133); an LCDR2 comprising ENDRRPS (SEQ ID NO: 134); and an LCDR3 comprising QSYDSSTHGWV (SEQ ID NO: 135);
(e) a V_{H} domain that comprises an HCDR1 comprising DYYMS (SEQ ID NO: 136); an HCDR2 comprising YTSRFGSDTNYADSVKG (SEQ ID NO: 137); and an HCDR3 comprising DVHNRDAY (SEQ ID NO: 138) and a (V_{L}) domain that comprises an LCDR1 comprising SGSSSNIGGNSVS (SEQ ID NO: 139); an LCDR2 comprising RNHQRPS (SEQ ID NO: 140); and an LCDR3 comprising ATWDFSLSGFV (SEQ ID NO: 141);
(f) a V_{H} domain that comprises an HCDR1 comprising SYAMS (SEQ ID NO: 142); an HCDR2 comprising AISGSGGSTYYADSVKG (SEQ ID NO: 143); and an HCDR3 comprising ADY (SEQ ID NO: 144) and a (V_{L}) domain that comprises an LCDR1 comprising RASQDIRNDLD (SEQ ID NO: 145); an LCDR2 comprising AASNLQS (SEQ ID NO: 146); and an LCDR3 comprising QQSYITPPWT (SEQ ID NO: 147);
(g) a V_{H} domain that comprises an HCDR1 comprising SYGMS (SEQ ID NO: 148); an HCDR2 comprising TISGSGSSTNYADSVKG (SEQ ID NO: 149); and an HCDR3 comprising GRYYYDSLDAFDI (SEQ ID NO: 150) and a (V_{L}) domain that comprises an LCDR1 comprising RASQEIRTAYLA (SEQ ID NO: 151); an LCDR2 comprising YASSRAT (SEQ ID NO: 152); and an LCDR3 comprising QQYDTSPPT (SEQ ID NO: 153);
(h) a V_{H} domain that comprises an HCDR1 comprising SYAMS (SEQ ID NO: 115); an HCDR2 comprising AISGTGGSTYYADSVKG (SEQ ID NO: 154); and an HCDR3 comprising DKWSSWPTYYFDY (SEQ ID NO: 155) and a (V_{L}) domain that comprises an LCDR1 comprising TRSSGSIASNYVQ (SEQ ID NO: 156); an LCDR2 comprising EDNQRPS (SEQ ID NO: 157); and an LCDR3 comprising QSYDSSNVI (SEQ ID NO: 158);
(i) a V_{H} domain that comprises an HCDR1 comprising SYSMA (SEQ ID NO: 159); an HCDR2 comprising AVSNSGVETYYADSVKG (SEQ ID NO: 160); and an HCDR3 comprising RTRQLLTPREFDY (SEQ ID NO: 161) and a (V_{L}) domain that comprises an LCDR1 comprising RASQDITRWLA (SEQ ID NO: 162); an LCDR2 comprising DASSLQS (SEQ ID NO: 163); and an LCDR3 comprising QQGSSVPFT (SEQ ID NO: 164);
(j) a V_{H} domain that comprises an HCDR1 comprising NYAMS (SEQ ID NO: 165); an HCDR2 comprising SVSSAGGSTYYADSVKG (SEQ ID NO: 166); and an HCDR3 comprising RVNRAFDL (SEQ ID NO: 167) and a (V_{L}) domain that comprises an LCDR1 comprising RASQSVSSSYLA (SEQ ID NO: 168); an LCDR2 comprising GASSRAT (SEQ ID NO: 169); and an LCDR3 comprising QQYGSSPPMYT (SEQ ID NO: 170);
(k) a V_{H} domain that comprises an HCDR1 comprising NAWMS (SEQ ID NO: 171); an HCDR2 comprising RIKSKTDGGTTDYAAPVKG (SEQ ID NO: 172); and an HCDR3 comprising DKSYGYTFDY (SEQ ID NO: 173) and a (V_{L}) domain that comprises an LCDR1 comprising SGSGSNIGSNSVH (SEQ ID NO: 174); an LCDR2 comprising TNNQRPS (SEQ ID NO: 175); and an LCDR3 comprising ATWDDRLSGPV (SEQ ID NO: 176);
(l) a V_{H} domain that comprises an HCDR1 comprising SYWMH (SEQ ID NO: 177); an HCDR2 comprising AISGSGAGTYYPDSVKG (SEQ ID NO: 178); and an HCDR3 comprising DRSLSFGFDI (SEQ ID NO: 179) and a (V_{L}) domain that comprises an LCDR1 comprising TRSSGSIGSTYVQ (SEQ ID NO: 180); an LCDR2 comprising KDDQRPS (SEQ ID NO: 181); and an LCDR3 comprising QSSDTSNLV (SEQ ID NO: 182);
(m) a V_{H} domain that comprises an HCDR1 comprising RYWMS (SEQ ID NO: 183); an HCDR2 comprising NIKGDGSQTYYADSVKG (SEQ ID NO: 184); and an HCDR3 comprising GAAYHINSWLDP (SEQ ID NO: 185) and a (V_{L}) domain that comprises an LCDR1 comprising RASQSISGNYLA (SEQ ID NO: 186); an LCDR2 comprising GAFRRAT (SEQ ID NO: 187); and an LCDR3 comprising QHYNNFPHT (SEQ ID NO: 188);
(n) a V_{H} domain that comprises an HCDR1 comprising HAWMN (SEQ ID NO: 189); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYQVNNRNYLA (SEQ ID NO: 190); an LCDR2 comprising QASTRAS (SEQ ID NO: 85); and an LCDR3 comprising QQYYTPPLA (SEQ ID NO: 86);
(o) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQTVLYPLNNRNYLA (SEQ ID NO: 97); an LCDR2 comprising QASTRAS (SEQ ID NO: 85); and an LCDR3 comprising QQYYTPPLA (SEQ ID NO: 86);
(p) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86);
(q) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 122) and a (V_{L}) domain that comprises an LCDR1 comprising KSSQSVLYPGNNRNYLA (SEQ ID NO: 191); an LCDR2 comprising QASTRAS (SEQ ID NO: 85); and an LCDR3 comprising QQYYTPPLA (SEQ ID NO: 86);
(r) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSSDI (SEQ ID NO: 192) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(s) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSSDI (SEQ ID NO: 193) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(t) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSSDI (SEQ ID NO: 194) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(u) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDA (SEQ ID NO: 195) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(v) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDS (SEQ ID NO: 196) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(w) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SDRASDK (SEQ ID NO: 98) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(x) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SAYAFDT (SEQ ID NO: 197) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(y) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GNHSQDI (SEQ ID NO: 198) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(z) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GQHSQDI (SEQ ID NO: 199)) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(aa) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising GAHSQDI (SEQ ID NO: 200) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(bb) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYYTPPLA (SEQ ID NO: 86);
(cc) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLRPPLN (SEQ ID NO: 202);
(dd) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLTPPLN (SEQ ID NO: 99);
(ee) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLS (SEQ ID NO: 203);
(ff) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLA (SEQ ID NO: 204);
(gg) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLNAPLH (SEQ ID NO: 205);
(hh) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLEAPLV (SEQ ID NO: 206);
(ii) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QQYLKAPLH (SEQ ID NO: 207);
(jj) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QRLIAPPFT (SEQ ID NO: 208);
(kk) a V_{H} domain that comprises an HCDR1 comprising NAWMN (SEQ ID NO: 121); an HCDR2 comprising RIKRKTDGETTDYAAPVKG (SEQ ID NO: 82); and an HCDR3 comprising SNRAFDI (SEQ ID NO: 201) and a (V_{L}) domain that comprises an LCDR1 comprising KSSQSVLYSSNNRNYLA (SEQ ID NO: 123); an LCDR2 comprising QASTRAS (SEQ ID NO: 85); and an LCDR3 comprising QNYLTPPLA (SEQ ID NO: 209);
(ll) a V_{H} domain that comprises an HCDR1 comprising SYYMH (SEQ ID NO: 210); an HCDR2 comprising EINPNNARINFNEKFKT (SEQ ID NO: 211); and an HCDR3 comprising GYYRYGAWFGY (SEQ ID NO: 212) and a (V_{L}) domain that comprises an LCDR1 comprising RASQDISDYLN (SEQ ID NO: 213); an LCDR2 comprising YISRLHS (SEQ ID NO: 214); and an LCDR3 comprising QQGHTLPWT (SEQ ID NO: 215);
or
(mm) V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86).

23. The method of claim 22, wherein the therapeutic anti-CD47 antibody comprises a V_{H} domain that comprises an HCDR1 comprising RAWMN (SEQ ID NO: 81); an HCDR2 comprising RIKRKTDGETTDYAAPVKG (SEQ ID NO: 82); and an HCDR3 comprising SNRAFDI (SEQ ID NO: 83) and a (V_{L}) domain that comprises an LCDR1 comprising KSSQSVLYAGNNRNYLA (SEQ ID NO: 84); an LCDR2 comprising QASTRAS (SEQ ID NO: 85); and an LCDR3 comprising QQYYTPPLA (SEQ ID NO: 86).

24. The method of claim 21, wherein the therapeutic anti-CD47 antibody comprises:
(a) a V_{H} that comprises SEQ ID NO: 1 and a V_{L} that comprises SEQ ID NO: 2;
(b) a V_{H} that comprises SEQ ID NO: 3 and a V_{L} that comprises SEQ ID NO: 4;
(c) a V_{H} that comprises SEQ ID NO: 5 and a V_{L} that comprises SEQ ID NO: 6;
(d) a V_{H} that comprises SEQ ID NO: 7 and a V_{L} that comprises SEQ ID NO: 8;
(e) a V_{H} that comprises SEQ ID NO: 9 and a V_{L} that comprises SEQ ID NO: 10;
(f) a V_{H} that comprises SEQ ID NO: 11 and a V_{L} that comprises SEQ ID NO: 12;
(g) a V_{H} that comprises SEQ ID NO: 13 and a V_{L} that comprises SEQ ID NO: 14;
(h) a V_{H} that comprises SEQ ID NO: 14 and a V_{L} that comprises SEQ ID NO: 15;
(i) a V_{H} that comprises SEQ ID NO: 16 and a V_{L} that comprises SEQ ID NO: 17;
(j) a V_{H} that comprises SEQ ID NO: 18 and a V_{L} that comprises SEQ ID NO: 19;
(k) a VH that comprises SEQ ID NO: 20 and a V_{L} that comprises SEQ ID NO: 21;
(l) a VH that comprises SEQ ID NO: 22 and a V_{L} that comprises SEQ ID NO: 23;
(m) a VH that comprises SEQ ID NO: 23 and a V_{L} that comprises SEQ ID NO: 24;
(n) a VH that comprises SEQ ID NO: 25 and a V_{L} that comprises SEQ ID NO: 26;
(o) a VH that comprises SEQ ID NO: 27 and a V_{L} that comprises SEQ ID NO: 28;
(p) a VH that comprises SEQ ID NO: 29 and a V_{L} that comprises SEQ ID NO: 30;
(q) a V_{H} that comprises SEQ ID NO: 31 and a V_{L} that comprises SEQ ID NO: 32;
(r) a VH that comprises SEQ ID NO: 33 and a V_{L} that comprises SEQ ID NO: 34;
(s) a VH that comprises SEQ ID NO: 35 and a V_{L} that comprises SEQ ID NO: 36;
(t) a VH that comprises SEQ ID NO: 37 and a V_{L} that comprises SEQ ID NO: 38;
(u) a VH that comprises SEQ ID NO: 39 and a V_{L} that comprises SEQ ID NO: 40;
(v) a VH that comprises SEQ ID NO: 41 and a V_{L} that comprises SEQ ID NO: 42;
(w) a VH that comprises SEQ ID NO: 43 and a V_{L} that comprises SEQ ID NO: 44;
(x) a VH that comprises SEQ ID NO: 45 and a V_{L} that comprises SEQ ID NO: 46;
(y) a VH that comprises SEQ ID NO: 47 and a V_{L} that comprises SEQ ID NO: 48;
(z) a VH that comprises SEQ ID NO: 49 and a V_{L} that comprises SEQ ID NO: 50;
(aa) a VH that comprises SEQ ID NO: 51 and a V_{L} that comprises SEQ ID NO: 52;
(bb) a VH that comprises SEQ ID NO: 53 and a V_{L} that comprises SEQ ID NO: 54;
(cc) a VH that comprises SEQ ID NO: 55 and a V_{L} that comprises SEQ ID NO: 56;
(dd) a VH that comprises SEQ ID NO: 57 and a V_{L} that comprises SEQ ID NO: 58;
(ee) a VH that comprises SEQ ID NO: 59 and a V_{L} that comprises SEQ ID NO: 60;
(ff) a VH that comprises SEQ ID NO: 61 and a V_{L} that comprises SEQ ID NO: 62;
(gg) a VH that comprises SEQ ID NO: 63 and a V_{L} that comprises SEQ ID NO: 64;
(hh) a VH that comprises SEQ ID NO: 65 and a V_{L} that comprises SEQ ID NO: 66;
(ii) a VH that comprises SEQ ID NO: 67 and a V_{L} that comprises SEQ ID NO: 68;
(jj) a VH that comprises SEQ ID NO: 69 and a V_{L} that comprises SEQ ID NO: 70;
(kk) a VH that comprises SEQ ID NO: 71 and a V_{L} that comprises SEQ ID NO: 72;
(ll) a VH that comprises SEQ ID NO: 73 and a V_{L} that comprises SEQ ID NO: 74;
(mm) a V_{H} that comprises SEQ ID NO: 75 and a V_{L} that comprises SEQ ID NO: 76;
(nn) a VH that comprises SEQ ID NO: 77 and a V_{L} that comprises SEQ ID NO: 78; or
(oo) a VH that comprises SEQ ID NO: 79 and a V_{L} that comprises SEQ ID NO: 80.

25. The method of claim 24, wherein the therapeutic anti-CD47 antibody comprises a V_{H} that comprises SEQ ID NO: 79 and a V_{L} that comprises SEQ ID NO: 80.

26. The method of any one of claims 1-25, wherein the wherein the therapeutic anti-CD47 antibody comprises a heavy chain comprising the amino acid sequence of SEQ ID NO: 216 or 217 and a light chain comprising the amino acid sequence of SEQ ID NO: 218.

27. The method of any one of claims 1-26, wherein the subject is diagnosed with cancer.

28. The method of any one of claims 1-27, wherein the cancer is a hematological malignancy.

29. The method of any one of claims 1-27, wherein the cancer is solid tumor

30. The method of any one of claims 2-29, wherein the method comprises a step of adding an enhancer to the blood sample prior to performing the serological assay.

31. The method of claim 30, wherein the enhancer is selected from the group consisting of: low ionic strength saline (LISS), polyethylene glycol (PEG), saline, and albumin.

32. The method of claim 31, wherein the enhancer is LISS.

33. The method of any one of claims 1-32, wherein the blood sample is selected from the group consisting of: a non-hemolyzed blood sample, a plasma sample, clotted blood, and serum.

34. The method of claim 33, wherein the blood sample is a plasma sample.

35. The method of claims 2-34, wherein the method further comprises a step of treating the blood sample with EDTA prior to performing the serological assay.

36. A method of transfusing donor blood to a subject who is under treatment with an anti-CD47 antibody, the method comprising:
(a) conducting the method of any one of claims 2-33 on a blood sample from the subject; and
(b) transfusing the donor blood into the individual,
wherein the donor blood is determined to be compatible with the individual according to the method of any one of claims 2-33.

37. The method of any one of claims 2-36, wherein the individual has anemia.

38. The method of claim 37, wherein the anemia is induced by the anti-CD47 antibody administered to the individual.

39. The method of any one of claims 34-38, wherein the blood transfusion is carried out within about 3 days after the administration of the anti-CD47 antibody.

40. The method of any one of claims 34-39, wherein the transfusing step is carried out within about 96 hours after the serological assay.

41. An anti-idiotypic antibody or immunologically active fragment thereof that specifically recognizes the antigen binding portion of a therapeutic anti-CD47 antibody, wherein the anti-idiotypic antibody comprises:
(a) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO:104); an LCDR2 comprising AASNLES (SEQ ID NO:105); and an LCDR3 comprising HQTNEDPWT (SEQ ID NO:106);
(b) a V_{H} domain that comprises an HCDR1 comprising NYGMN (SEQ ID NO: 101); an HCDR2 comprising WINTYTGQPTHADDFKG (SEQ ID NO: 102); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising HQTNEDPWT (SEQ ID NO:109);
(c) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising AASNLES (SEQ ID NO: 105); and an LCDR3 comprising QQTHEDPWT (SEQ ID NO: 220);
(d) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising LVSNLES (SEQ ID NO: 108); and an LCDR3 comprising QQTHEDPWT (SEQ ID NO: 220);
(e) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising RASKSVSTSGYSYMH (SEQ ID NO: 107); an LCDR2 comprising AASNLES (SEQ ID NO: 105); and an LCDR3 comprising QQTHEDPWT (SEQ ID NO: 220);
(f) a V_{H} domain that comprises an HCDR1 comprising NYGMN (SEQ ID NO: 101); an HCDR2 comprising WINTFTGEPTLADDFMG (SEQ ID NO: 219); and an HCDR3 comprising GGMGVRLRYFDV (SEQ ID NO: 103); and a light chain variable (V_{L}) domain that comprises an LCDR1 comprising KASQSVDYDGDSYMD (SEQ ID NO: 104); an LCDR2 comprising LVSNLES (SEQ ID NO: 108); and an LCDR3 comprising QQTHEDPWT (SEQ ID NO: 220);
or
(g) a V_{H} domain that comprises an HCDR1 comprising DYNMN (SEQ ID NO: 100); an HCDR2 comprising YVDPYYGDTRYNQNFKG (SEQ ID NO: 235); and an HCDR3 comprising SETPRAMDY (SEQ ID NO: 236); and a light chain variable (V_{L}) domain that comprises an LCDR1 comprising RASQSISDYLH (SEQ ID NO: 237); an LCDR2 comprising YASQSIS (SEQ ID NO: 238); and an LCDR3 comprising QNGHSLPLT (SEQ ID NO: 239).

42. The anti-idiotypic antibody or immunologically active fragment thereof of claim 41, wherein the anti-idiotypic antibody comprises:
(a) a V_{H} comprising SEQ ID NO: 110 and a V_{L} comprising SEQ ID NO: 111;
(b) a V_{H} comprising SEQ ID NO: 110 and a V_{L} comprising SEQ ID NO: 112;
(c) a V_{H} comprising SEQ ID NO: 110 and a V_{L} comprising SEQ ID NO: 113;
(d) a V_{H} comprising SEQ ID NO: 110 and a V_{L} comprising SEQ ID NO: 114;
(e) a V_{H} comprising SEQ ID NO: 95 and a V_{L} comprising SEQ ID NO: 87;
(f) a V_{H} comprising SEQ ID NO: 95 and a V_{L} comprising SEQ ID NO: 88;
(g) a V_{H} comprising SEQ ID NO: 95 and a V_{L} comprising SEQ ID NO: 89;
(h) a V_{H} comprising SEQ ID NO: 95 and a V_{L} comprising SEQ ID NO: 90;
(i) a V_{H} comprising SEQ ID NO: 95 and a V_{L} comprising SEQ ID NO: 91;
(j) a V_{H} comprising SEQ ID NO: 95 and a V_{L} comprising SEQ ID NO: 92; or
(k) a V_{H} comprising SEQ ID NO: 93 and a V_{L} comprising SEQ ID NO: 94.

43. A method of detecting the presence of an anti-CD47 antibody or immunologically active fragment thereof in a sample of an individual, comprising:
(a) contacting the sample with the anti-idiotypic antibody of claim 41 or 42, and
(b) detecting a complex comprising the anti-idiotypic antibody and the anti-CD47 antibody or fragment thereof, thereby detecting the presence of anti-CD47 antibody or fragment thereof.
